SEQUENCE LISTING (376 sequences)

SEQ ID NO: 1 - protein, 879 aa, Homo sapiens
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2 - protein, 731 aa, Homo sapiens
PSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 3 - protein, 611 aa, Homo sapiens
VATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 4 - protein, 485 aa, Homo sapiens
SPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 5 - protein, 343 aa, Homo sapiens
KPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 6 - protein, 217 aa, Homo sapiens
VRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 7 - protein, 66 aa, Homo sapiens
SKPVFITVKMDVLNAFKYPLLIGVGLSTVIGLLSCLIGYCSSHWCCKKEVQETRRERRRLMSMEMD

SEQ ID NO: 8 - protein, 21 aa, Homo sapiens
MGRLASRPLLLALLSLALCRG

SEQ ID NO: 9 - protein, 385 aa, Homo sapiens
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 10 - protein, 247 aa, Homo sapiens
PGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 11 - protein, 168 aa, Homo sapiens
HGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 12 - protein, 66 aa, Homo sapiens
SHLAALWPFLGIVAEVLVLVTIIFIYEKRRKPEDVLDDDDAGSAPLKSSGQHQNDKGKNVRQRNSS

SEQ ID NO: 13 - protein, 18 aa, Homo sapiens
MAAALFVLLGFALLGTHG

SEQ ID NO: 14 - protein, 613 aa, Homo sapiens
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 15 - protein, 456 aa, Homo sapiens
APPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 16 - protein, 320 aa, Homo sapiens
AHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 17 - protein, 179 aa, Homo sapiens
REEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 18 - protein, 24 aa, Homo sapiens
VALVTGATVLGTITCCFMKRLRKR

SEQ ID NO: 19 - protein, 27 aa, Homo sapiens
MGALRPTLLPPSLPLLLLLMLGMGCWA

SEQ ID NO: 20 - protein, 1195 aa, Homo sapiens
MKCFFPVLSCLAVLGVVSAQRQVTVQEGPLYRTEGSHITIWCNVSGYQGPSEQNFQWSIYLPSSPEREVQIVSTMDSSFPYAIYTQRVRGGKIFIERVQGNSTLLHITDLQARDAGEYECHTPSTDKQYFGSYSAKMNLVVIPDSLQTTAMPQTLHRVEQDPLELTCEVASETIQHSHLSVAWLRQKVGEKPVEVISLSRDFMLHSSSEYAQRQSLGEVRLDKLGRTTFRLTIFHLQPSDQGEFYCEAAEWIQDPDGSWYAMTRKRSEGAVVNVQPTDKEFTVRLETEKRLHTVGEPVEFRCILEAQNVPDRYFAVSWAFNSSLIATMGPNAVPVLNSEFAHREARGQLKVAKESDSVFVLKIYHLRQEDSGKYNCRVTEREKTVTGEFIDKESKRPKNIPIIVLPLKSSISVEVASNASVILEGEDLRFSCSVRTAGRPQGRFSVIWQLVDRQNRRSNIMWLDRDGTVQPGSSYWERSSFGGVQMEQVQPNSFSLGIFNSRKEDEGQYECHVTEWVRAVDGEWQIVGERRASTPISITALEMGFAVTAISRTPGVTYSDSFDLQCIIKPHYPAWVPVSVTWRFQPVGTVEFHDLVTFTRDGGVQWGDRSSSFRTRTAIEKAESSNNVRLSISRASDTEAGKYQCVAELWRKNYNNTWTRLAERTSNLLEIRVLQPVTKLQVSKSKRTLTLVENKPIQLNCSVKSQTSQNSHFAVLWYVHKPSDADGKLILKTTHNSAFEYGTYAEEEGLRARLQFERHVSGGLFSLTVQRAEVSDSGSYYCHVEEWLLSPNYAWYKLAEEVSGRTEVTVKQPDSRLRLSQAQGNLSVLETRQVQLECVVLNRTSITSQLMVEWFVWKPNHPERETVARLSRDATFHYGEQAAKNNLKGRLHLESPSPGVYRLFIQNVAVQDSGTYSCHVEEWLPSPSGMWYKRAEDTAGQTALTVMRPDASLQVDTVVPNATVSEKAAFQLDCSIVSRSSQDSRFAVAWYSLRTKAGGKRSSPGLEEQEEEREEEEEEEEDDDDDDPTERTALLSVGPDAVFGPEGSPWEGRLRFQRLSPVLYRLTVLQASPQDTGNYSCHVEEWLPSPQKEWYRLTEEESAPIGIRVLDTSPTLQSIICSNDALFYFVFFYPFPIFGILIITILLVRFKSRNSSKNSDGKNGVPLLWIKEPHLNYSPTCLEPPVLSIHPGAID

SEQ ID NO: 21 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 22 - protein, 1032 aa, Homo sapiens [sequence omitted]

SEQ ID NO: 23 - protein, 660 aa, Homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTETGFHHVSQADIEFLTSIDPTASASGSAGITGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYA

SEQ ID NO: 24 - protein, 1023 aa, Homo sapiens
MGKGVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 25 - protein, 1020 aa, Homo sapiens
MGRGAGREYSPAATTAENGGGKKKQKEKELDELKKEVAMDDHKLSLDELGRKYQVDLSKGLTNQRAQDVLARDGPNALTPPPTTPEWVKFCRQLFGGFSILLWIGAILCFLAYGIQAAMEDEPSNDNLYLGVVLAAVVIVTGCFSYYQEAKSSKIMDSFKNMVPQQALVIREGEKMQINAEEVVVGDLVEVKGGDRVPADLRIISSHGCKVDNSSLTGESEPQTRSPEFTHENPLETRNICFFSTNCVEGTARGIVIATGDRTVMGRIATLASGLEVGRTPIAMEIEHFIQLITGVAVFLGVSFFVLSLILGYSWLEAVIFLIGIIVANVPEGLLATVTVCLTLTAKRMARKNCLVKNLEAVETLGSTSTICSDKTGTLTQNRMTVAHMWFDNQIHEADTTEDQSGATFDKRSPTWTALSRIAGLCNRAVFKAGQENISVSKRDTAGDASESALLKCIELSCGSVRKMRDRNPKVAEIPFNSTNKYQLSIHEREDSPQSHVLVMKGAPERILDRCSTILVQGKEIPLDKEMQDAFQNAYMELGGLGERVLGFCQLNLPSGKFPRGFKFDTDELNFPTEKLCFVGLMSMIDPPRAAVPDAVGKCRSAGIKVIMVTGDHPITAKAIAKGVGIISEGNETVEDIAARLNIPMSQVNPREAKACVVHGSDLKDMTSEQLDEILKNHTEIVFARTSPQQKLIIVEGCQRQGAIVAVTGDGVNDSPALKKADIGIAMGISGSDVSKQAADMILLDDNFASIVTGVEEGRLIFDNLKKSIAYTLTSNIPEITPFLLFIIANIPLPLGTVTILCIDLGTDMVPAISLAYEAAESDIMKRQPRNSQTDKLVNERLISMAYGQIGMIQALGGFFTYFVILAENGFLPSRLLGIRLDWDDRTMNDLEDSYGQEWTYEQRKVVEFTCHTAFFASIVVVQWADLIICKTRRNSVFQQGMKNKILIFGLLEETALAAFLSYCPGMGVALRMYPLKVTWWFCAFPYSLLIFIYDEVRKLILRRYPGGWVEKETYY

SEQ ID NO: 26 - protein, 1026 aa, Homo sapiens
MGSGGSDSYRIATSQDKKDDKDSPKKNKGKERRDLDDLKKEVAMTEHKMSVEEVCRKYNTDCVQGLTHSKAQEILARDGPNALTPPPTTPEWVKFCRQLFGGFSILLWIGAILCFLAYGIQAGTEDDPSGDNLYLGIVLAAVVIITGCFSYYQEAKSSKIMESFKNMVPQQALVIREGEKMQVNAEEVVVGDLVEIKGGDRVPADLRIISAHGCKVDNSSLTGESEPQTRSPDCTHDNPLETRNITFFSTNCVEGTARGVVVATGDRTVMGRIATLASGLEVGKTPIAIEIEHFIQLITGVAVFLGVSFFILSLILGYTWLEAVIFLIGIIVANVPEGLLATVTVCLTLTAKRMARKNCLVKNLEAVETLGSTSTICSDKTGTLTQNRMTVAHMWFDNQIHEADTTEDQSGTSFDKSSHTWVALSHIAGLCNRAVFKGGQDNIPVLKRDVAGDASESALLKCIELSSGSVKLMRERNKKVAEIPFNSTNKYQLSIHETEDPNDNRYLLVMKGAPERILDRCSTILLQGKEQPLDEEMKEAFQNAYLELGGLGERVLGFCHYYLPEEQFPKGFAFDCDDVNFTTDNLCFVGLMSMIDPPRAAVPDAVGKCRSAGIKVIMVTGDHPITAKAIAKGVGIISEGNETVEDIAARLNIPVSQVNPRDAKACVIHGTDLKDFTSEQIDEILQNHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLLFIMANIPLPLGTITILCIDLGTDMVPAISLAYEAAESDIMKRQPRNPRTDKLVNERLISMAYGQIGMIQALGGFFSYFVILAENGFLPGNLVGIRLNWDDRTVNDLEDSYGQQWTYEQRKVVEFTCHTAFFVSIVVVQWADLIICKTRRNSVFQQGMKNKILIFGLFEETALAAFLSYCPGMDVALRMYPLKPSWWFCAFPYSFLIFVYDEIRKLILRRNPGGWVEKETYY

SEQ ID NO: 27 - protein, 1029 aa, Homo sapiens
MGLWGKKGTVAPHDQSPRRRPKKGLIKKKMVKREKQKRNMEELKKEVVMDDHKLTLEELSTKYSVDLTKGHSHQRAKEILTRGGPNTVTPPPTTPEWVKFCKQLFGGFSLLLWTGAILCFVAYSIQIYFNEEPTKDNLYLSIVLSVVVIVTGCFSYYQEAKSSKIMESFKNMVPQQALVIRGGEKMQINVQEVVLGDLVEIKGGDRVPADLRLISAQGCKVDNSSLTGESEPQSRSPDFTHENPLETRNICFFSTNCVEGTARGIVIATGDSTVMGRIASLTSGLAVGQTPIAAEIEHFIHLITVVAVFLGVTFFALSLLLGYGWLEAIIFLIGIIVANVPEGLLATVTVCLTLTAKRMARKNCLVKNLEAVETLGSTSTICSDKTGTLTQNRMTVAHMWFDMTVYEADTTEEQTGKTFTKSSDTWFMLARIAGLCNRADFKANQEILPIAKRATTGDASESALLKFIEQSYSSVAEMREKNPKVAEIPFNSTNKYQMSIHLREDSSQTHVLMMKGAPERILEFCSTFLLNGQEYSMNDEMKEAFQNAYLELGGLGERVLGFCFLNLPSSFSKGFPFNTDEINFPMDNLCFVGLISMIDPPRAAVPDAVSKCRSAGIKVIMVTGDHPITAKAIAKGVGIISEGTETAEEVAARLKIPISKVDASAAKAIVVHGAELKDIQSKQLDQILQNHPEIVFARTSPQQKLIIVEGCQRLGAVVAVTGDGVNDSPALKKADIGIAMGISGSDVSKQAADMILLDDNFASIVTGVEEGRLIFDNLKKSIMYTLTSNIPEITPFLMFIILGIPLPLGTITILCIDLGTDMVPAISLAYESAESDIMKRLPRNPKTDNLVNHRLIGMAYGQIGMIQALAGFFTYFVILAENGFRPVDLLGIRLHWEDKYLNDLEDSYGQQWTYEQRKVVEFTCQTAFFVTIVVVQWADLIISKTRRNSLFQQGMRNKVLIFGILEETLLAAFLSYTPGMDVALRMYPLKITWWLCAIPYSILIFVYDEIRKLLIRQHPDGWVERETYY

SEQ ID NO: 28 - protein, 279 aa, Homo sapiens
MTKNEKKSLNQSLAEWKLFIYNPTTGEFLGRTAKSWGLILLFYLVFYGFLAALFSFTMWVMLQTLNDEVPKYRDQIPSPGLMVFPKPVTALEYTFSRSDPTSYAGYIEDLKKFLKPYTLEEQKNLTVCPDGALFEQKGPVYVACQFPISLLQACSGMNDPDFGYSQGNPCILVKMNRIIGLKPEGVPRIDCVSKNEDIPNVAVYPHNGMIDLKYFPYYGKKLHVGYLQPLVAVQVSFAPNNTGKEVTVECKIDGSANLKSQDDRDKFLGRVMFKITARA

SEQ ID NO: 29 - protein, 1258 aa, Homo sapiens
MGDMANNSVAYSGVKNSLKEANHDGDFGITLAELRALMELRSTDALRKIQESYGDVYGICTKLKTSPNEGLSGNPADLERREAVFGKNFIPPKKPKTFLQLVWEALQDVTLIILEIAAIVSLGLSFYQPPEGDNALCGEVSVGEEEGEGETGWIEGAAILLSVVCVVLVTAFNDWSKEKQFRGLQSRIEQEQKFTVIRGGQVIQIPVADITVGDIAQVKYGDLLPADGILIQGNDLKIDESSLTGESDHVKKSLDKDPLLLSGTHVMEGSGRMVVTAVGVNSQTGIIFTLLGAGGEEEEKKDEKKKEKKNKKQDGAIENRNKAKAQDGAAMEMQPLKSEEGGDGDEKDKKKANLPKKEKSVLQGKLTKLAVQIGKAGLLMSAITVIILVLYFVIDTFWVQKRPWLAECTPIYIQYFVKFFIIGVTVLVVAVPEGLPLAVTISLAYSVKKMMKDNNLVRHLDACETMGNATAICSDKTGTLTMNRMTVVQAYINEKHYKKVPEPEAIPPNILSYLVTGISVNCAYTSKILPPEKEGGLPRHVGNKTECALLGLLLDLKRDYQDVRNEIPEEALYKVYTFNSVRKSMSTVLKNSDGSYRIFSKGASEIILKKCFKILSANGEAKVFRPRDRDDIVKTVIEPMASEGLRTICLAFRDFPAGEPEPEWDNENDIVTGLTCIAVVGIEDPVRPEVPDAIKKCQRAGITVRMVTGDNINTARAIATKCGILHPGEDFLCLEGKDFNRRIRNEKGEIEQERIDKIWPKLRVLARSSPTDKHTLVKGIIDSTVSDQRQVVAVTGDGTNDGPALKKADVGFAMGIAGTDVAKEASDIILTDDNFTSIVKAVMWGRNVYDSISKFLQFQLTVNVVAVIVAFTGACITQDSPLKAVQMLWVNLIMDTLASLALATEPPTESLLLRKPYGRNKPLISRTMMKNILGHAFYQLVVVFTLLFAGEKFFDIDSGRNAPLHAPPSEHYTIVFNTFVLMQLFNEINARKIHGERNVFEGIFNNAIFCTIVLGTFVVQIIIVQFGGKPFSCSELSIEQWLWSIFLGMGTLLWGQLISTIPTSRLKFLKEAGHGTQKEEIPEEELAEDVEEIDHAERELRRGQILWFRGLNRIQTQMDVVNAFQSGSSIQGALRRQPSIASQHHDVTNISTPTHIRVVNAFRSSLYEGLEKPESRSSIHNFMTHPEFRIEDSEPHIPLIDDTDAEDDAPTKRNSSPPPSPNKNNNAVDSGIHLTIEMNKSATSSSPGSPLHSLETSL

SEQ ID NO: 30 - protein, 1272 aa, Homo sapiens
MGDMTNSDFYSKNQRNESSHGGEFGCTMEELRSLMELRGTEAVVKIKETYGDTEAICRRLKTSPVEGLPGTAPDLEKRKQIFGQNFIPPKKPKTFLQLVWEALQDVTLIILEIAAIISLGLSFYHPPGEGNEGCATAQGGAEDEGEAEAGWIEGAAILLSVICVVLVTAFNDWSKEKQFRGLQSRIEQEQKFTVVRAGQVVQIPVAEIVVGDIAQVKYGDLLPADGLFIQGNDLKIDESSLTGESDQVRKSVDKDPMLLSGTHVMEGSGRMLVTAVGVNSQTGIIFTLLGAGGEEEEKKDKKGVKKGDGLQLPAADGAAASNAADSANASLVNGKMQDGNVDASQSKAKQQDGAAAMEMQPLKSAEGGDADDRKKASMHKKEKSVLQGKLTKLAVQIGKAGLVMSAITVIILVLYFTVDTFVVNKKPWLPECTPVYVQYFVKFFIIGVTVLVVAVPEGLPLAVTISLAYSVKKMMKDNNLVRHLDACETMGNATAICSDKTGTLTTNRMTVVQAYVGDVHYKEIPDPSSINTKTMELLINAIAINSAYTTKILPPEKEGALPRQVGNKTECGLLGFVLDLKQDYEPVRSQMPEEKLYKVYTFNSVRKSMSTVIKLPDESFRMYSKGASEIVLKKCCKILNGAGEPRVFRPRDRDEMVKKVIEPMACDGLRTICVAYRDFPSSPEPDWDNENDILNELTCICVVGIEDPVRPEVPEAIRKCQRAGITVRMVTGDNINTARAIAIKCGIIHPGEDFLCLEGKEFNRRIRNEKGEIEQERIDKIWPKLRVLARSSPTDKHTLVKGIIDSTHTEQRQVVAVTGDGTNDGPALKKADVGFAMGIAGTDVAKEASDIILTDDNFSSIVKAVMWGRNVYDSISKFLQFQLTVNVVAVIVAFTGACITQDSPLKAVQMLWVNLIMDTFASLALATEPPTETLLLRKPYGRNKPLISRTMMKNILGHAVYQLALIFTLLFVGEKMFQIDSGRNAPLHSPPSEHYTIIFNTFVMMQLFNEINARKIHGERNVFDGIFRNPIFCTIVLGTFAIQIVIVQFGGKPFSCSPLQLDQWMWCIFIGLGELVWGQVIATIPTSRLKFLKEAGRLTQKEEIPEEELNEDVEEIDHAERELRRGQILWFRGLNRIQTQIEVVNTFKSGASFQGALRRQSSVTSQSQDIRVVKAFRSSLYEGLEKPESRTSIHNFMAHPEFRIEDSQPHIPLIDDTDLEEDAALKQNSSPPSSLNKNNSAIDSGINLTTDTSKSATSSSPGSPIHSLETSL

SEQ ID NO: 31 - protein, 1241 aa, Homo sapiens
MTNPSDRVLPANSMAESREGDFGCTVMELRKLMELRSRDALTQINVHYGGVQNLCSRLKTSPVEGLSGNPADLEKRRQVFGHNVIPPKKPKTFLELVWEALQDVTLIILEIAAIISLVLSFYRPAGEENELCGQVATTPEDENEAQAGWIEGAAILFSVIIVVLVTAFNDWSKEKQFRGLQCRIEQEQKFSIIRNGQLIQLPVAEIVVGDIAQVKYGDLLPADGILIQGNDLKIDESSLTGESDHVKKSLDKDPMLLSGTHVMEGSGRMVVTAVGVNSQTGIILTLLGVNEDDEGEKKKKGKKQGVPENRNKAKTQDGVALEIQPLNSQEGIDNEEKDKKAVKVPKKEKSVLQGKLTRLAVQIGKAGLLMSALTVFILILYFVIDNFVINRRPWLPECTPIYIQYFVKFFIIGITVLVVAVPEGLPLAVTISLAYSVKKMMKDNNLVRHLDACETMGNATAICSDKTGTLTMNRMTVVQAYIGGIHYRQIPSPDVFLPKVLDLIVNGISINSAYTSKILPPEKEGGLPRQVGNKTECALLGFVTDLKQDYQAVRNEVPEEKLYKVYTFNSVRKSMSTVIRNPNGGFRMYSKGASEIILRKCNRILDRKGEAVPFKNKDRDDMVRTVIEPMACDGLRTICIAYRDFDDTEPSWDNENEILTELTCIAVVGIEDPVRPEVPDAIAKCKQAGITVRMVTGDNINTARAIATKCGILTPGDDFLCLEGKEFNRLIRNEKGEVEQEKLDKIWPKLRVLARSSPTDKHTLVKGIIDSTVGEHRQVVAVTGDGTNDGPALKKADVGFAMGIAGTDVAKEASDIILTDDNFTSIVKAVMWGRNVYDSISKFLQFQLTVNVVAVIVAFTGACITQDSPLKAVQMLWVNLIMDTFASLALATEPPTESLLKRRPYGRNKPLISRTMMKNILGHAFYQLIVIFILVFAGEKFFDIDSGRKAPLHSPPSQHYTIVFNTFVLMQLFNEINSRKIHGEKNVFSGIYRNIIFCSVVLGTFICQIFIVEFGGKPFSCTSLSLSQWLWCLFIGIGELLWGQFISAIPTRSLKFLKEAGHGTTKEEITKDAEGLDEIDHAEMELRRGQILWFRGLNRIQTQIDVINTFQTGASFKGVLRRQNMGQHLDVKLVPSSSYIKVVKAFHSSLHESIQKPYNQKSIHSFMTHPEFAIEEELPRTPLLDEEEEENPDKASKFGTRVLLLDGEVTPYANTNNNAVDCNQVQLPQSDSSLQSLETSV

SEQ ID NO: 32 - protein, 1241 aa, Homo sapiens; identical to SEQ ID NO: 31

SEQ ID NO: 33 - protein, 193 aa, Homo sapiens
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 34 - protein, 1021 aa, Homo sapiens
MAGISYVASFFLLLTKLSIGQREVTVQKGPLFRAEGYPVSIGCNVTGHQGPSEQHFQWSVYLPTNPTQEVQIISTKDAAFSYAVYTQRVRSGDVYVERVQGNSVLLHISKLQMKDAGEYECHTPNTDEKYYGSYSAKTNLIVIPDTLSATMSSQTLGKEEGEPLALTCEASKATAQHTHLSVTWYLTQDGGGSQATEIISLSKDFILVPGPLYTERFAASDVQLNKLGPTTFRLSIERLQSSDQGQLFCEATEWIQDPDETWMFITKKQTDQTTLRIQPAVKDFQVNITADSLFAEGKPLELVCLVVSSGRDPQLQGIWFFNGTEIAHIDAGGVLGLKNDYKERASQGELQVSKLGPKAFSLKIFSLGPEDEGAYRCVVAEVMKTRTGSWQVLQRKQSPDSHVHLRKPAARSVVMSTKNKQQVVWEGETLAFLCKAGGAESPLSVSWWHIPRDQTQPEFVAGMGQDGIVQLGASYGVPSYHGNTRLEKMDWATFQLEITFTAITDSGTYECRVSEKSRNQARDLSWTQKISVTVKSLESSLQVSLMSRQPQVMLTNTFDLSCVVRAGYSDLKVPLTVTWQFQPASSHIFHQLIRITHNGTIEWGNFLSRFQKKTKVSQSLFRSQLLVHDATEEETGVYQCEVEVYDRNSLYNNRPPRASAISHPLRIAVTLPESKLKVNSRSQVQELSINSNTDIECSILSRSNGNLQLAIIWYFSPVSTNASWLKILEMDQTNVIKTGDEFHTPQRKQKFHTEKVSQDLFQLHILNVEDSDRGKYHCAVEEWLLSTNGTWHKLGEKKSGLTELKLKPTGSKVRVSKVYWTENVTEHREVAIRCSLESVGSSATLYSVMWYWNRENSGSKLLVHLQHDGLLEYGEEGLRRHLHCYRSSSTDFVLKLHQVEMEDAGMYWCRVAEWQLHGHPSKWINQASDESQRMVLTVLPSEPTLPSRICSSAPLLYFLFICPFVLLLLLLISLLCLYWKARKLSTLRSNTRKEKALWVDLKEAGGVTTNRREDEEEDEGN

SEQ ID NO: 35 - protein, 20 aa, Homo sapiens
MAGISYVASFFLLLTKLSIG

SEQ ID NO: 36 - DNA, 20 bp, synthetic construct
  misc_feature 1..20: note = Synthetic Oligonucleotide
  source 1..20: mol_type = other DNA
cgttggcagtccgccttaac

SEQ ID NO: 37 - DNA, 20 bp, synthetic construct
  misc_feature 1..20: note = Synthetic oligonucleotide
  source 1..20: mol_type = other DNA
catagtcactgacgttgcag

SEQ ID NO: 38 - DNA, 20 bp, synthetic construct
  misc_feature 1..20: note = Synthetic oligonucleotide
  source 1..20: mol_type = other DNA
ttgtggagcttgcaagcacc

SEQ ID NO: 39 - DNA, 20 bp, synthetic construct
  misc_feature 1..20: note = Synthetic oligonucleotide
  source 1..20: mol_type = other DNA
gttctttatgtggagctcca

SEQ ID NO: 40 - DNA, 20 bp, synthetic construct
  misc_feature 1..20: note = Synthetic oligonucleotide
  source 1..20: mol_type = other DNA
tatcccttgctgatcggcgt

SEQ ID NO: 41 - DNA, 20 bp, synthetic construct
  misc_feature 1..20: note = Synthetic oligonucleotide
  source 1..20: mol_type = other DNA
gctgcagtacccgatgagac

SEQ ID NO: 42 - protein, 38 aa, synthetic construct
  REGION 1..38: note = Synthetic Polypeptide
EHSAGGGGSDYKDDDDKGGGGSLSNPIEIDFQTSGPIF

SEQ ID NO: 43 - protein, 34 aa, synthetic construct
  REGION 1..34: note = Synthetic Polypeptide
EHSAGGGGSDYKDDDDKGGGGSIEIDFQTSGPIF

SEQ ID NO: 44 - protein, 30 aa, synthetic construct
  REGION 1..30: note = Synthetic Polypeptide
EHSAGGGGSDYKDDDDKGGGGSFQTSGPIF

SEQ ID NO: 45 - protein, 26 aa, synthetic construct
  REGION 1..26: note = Synthetic Polypeptide
EHSAGGGGSDYKDDDDKGGGGSGPIF

SEQ ID NO: 46 - protein, 42 aa, synthetic construct
  REGION 1..42: note = Synthetic Polypeptide
FITVKMDTLDPRSFLLRNPNDKYEPFWEDEEKNESGSDKTHT

SEQ ID NO: 47 - protein, 332 aa, Homo sapiens
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 48 - protein, 195 aa, Homo sapiens
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 49 - protein, 227 aa, Homo sapiens
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 50 - protein, 30 aa, synthetic construct
  REGION 1..30: note = Synthetic peptide
MGGKLSKKKKGYNVNDEKAKEKDKKAEGAA

SEQ ID NO: 51 - protein, 27 aa, synthetic construct
  REGION 1..27: note = Synthetic peptide
MGGKLSKKKKGYNVNDEKAKEKDKKAE

SEQ ID NO: 52 - protein, 24 aa, synthetic construct
  REGION 1..24: note = Synthetic peptide
MGGKLSKKKKGYNVNDEKAKEKDK

SEQ ID NO: 53 - protein, 21 aa, synthetic construct
  REGION 1..21: note = Synthetic peptide
MGGKLSKKKKGYNVNDEKAKE

SEQ ID NO: 54 - protein, 18 aa, synthetic construct
  REGION 1..18: note = Synthetic peptide
MGGKLSKKKKGYNVNDEK

SEQ ID NO: 55 - protein, 15 aa, synthetic construct
  REGION 1..15: note = Synthetic peptide
MGGKLSKKKKGYNVN

SEQ ID NO: 56 - protein, 12 aa, synthetic construct
  REGION 1..12: note = Synthetic peptide
MGGKLSKKKKGY

SEQ ID NO: 57 - protein, 11 aa, synthetic construct
  REGION 1..11: note = Synthetic peptide
MGGKLSKKKKG

SEQ ID NO: 58 - protein, 10 aa, synthetic construct
  REGION 1..10: note = Synthetic peptide
MGGKLSKKKK

SEQ ID NO: 59 - protein, 9 aa, synthetic construct
  REGION 1..9: note = Synthetic peptide
MGGKLSKKK

SEQ ID NO: 60 - protein, 8 aa, synthetic construct
  REGION 1..8: note = Synthetic peptide
MGGKLSKK

SEQ ID NO: 61 - protein, 7 aa, synthetic construct
  REGION 1..7: note = Synthetic peptide
MGGKLSK

SEQ ID NO: 62 - protein, 8 aa, synthetic construct
  REGION 1..8: note = Synthetic peptide
MGGKLAKK

SEQ ID NO: 63 - protein, 8 aa, synthetic construct
  REGION 1..8: note = Synthetic peptide
MGGKFSKK

SEQ ID NO: 64 - protein, 8 aa, synthetic construct
  REGION 1..8: note = Synthetic peptide
MGGKFAKK

SEQ ID NO: 65 - protein, 8 aa, synthetic construct
  REGION 1..8: note = Synthetic peptide
MGGKSSKK

SEQ ID NO: 66 - protein, 8 aa, synthetic construct
  REGION 1..8: note = Synthetic peptide
MGGKSAKK

SEQ ID NO: 67 - protein, 8 aa, synthetic construct
  REGION 1..8: note = Synthetic peptide
MGGKQSKK

SEQ ID NO: 68 - protein, 8 aa, synthetic construct
  REGION 1..8: note = Synthetic peptide
MGGKQAKK

SEQ ID NO: 69 - protein, 8 aa, synthetic construct
  REGION 1..8: note = Synthetic peptide
MGGQLSKK

SEQ ID NO: 70 - protein, 8 aa, synthetic construct
  REGION 1..8: note = Synthetic peptide
MGGQLAKK

SEQ ID NO: 71 - protein, 8 aa, synthetic construct
  REGION 1..8: note = Synthetic peptide
MGGQFSKK

SEQ ID NO: 72 - protein, 8 aa, synthetic construct
  REGION 1..8: note = Synthetic peptide
MGGQFAKK

SEQ ID NO: 73 - protein, 8 aa, synthetic construct
  REGION 1..8: note = Synthetic peptide
MGGQSSKK

SEQ ID NO: 74 - protein, 8 aa, synthetic construct
  REGION 1..8: note = Synthetic peptide
MGGQSAKK

SEQ ID NO: 75 - protein, 8 aa, synthetic construct
  REGION 1..8: note = Synthetic peptide
MGGQQSKK

SEQ ID NO: 76 - protein, 8 aa, synthetic construct
  REGION 1..8: note = Synthetic peptide
MGGQQAKK

SEQ ID NO: 77 - protein, 8 aa, synthetic construct
  REGION 1..8: note = Synthetic peptide
MGAKLSKK

SEQ ID NO: 78 - protein, 8 aa, synthetic construct
  REGION 1..8: note = Synthetic peptide
MGAKLAKK

SEQ ID NO: 79 - protein, 8 aa, synthetic construct
  REGION 1..8: note = Synthetic peptide
MGAKFSKK

SEQ ID NO: 80 - protein, 8 aa, synthetic construct
  REGION 1..8: note = Synthetic peptide
MGAKFAKK

SEQ ID NO: 81 - protein, 8 aa, synthetic construct
  REGION 1..8: note = Synthetic peptide
MGAKSSKK

SEQ ID NO: 82 - protein, 8 aa, synthetic construct
  REGION 1..8: note = Synthetic peptide
MGAKSAKK

SEQ ID NO: 83 - protein, 8 aa, synthetic construct
  REGION 1..8: note = Synthetic peptide
MGAKQSKK

SEQ ID NO: 84 - protein, 8 aa, synthetic construct
  REGION 1..8: note = Synthetic peptide
MGAKQAKK

SEQ ID NO: 85 - protein, 8 aa, synthetic construct
  REGION 1..8: note = Synthetic peptide
MGAQLSKK

SEQ ID NO: 86 - protein, 8 aa, synthetic construct
  REGION 1..8: note = Synthetic peptide
MGAQLAKK

SEQ ID NO: 87 - protein, 8 aa, synthetic construct
  REGION 1..8: note = Synthetic peptide
MGAQFSKK

SEQ ID NO: 88 - protein, 8 aa, synthetic construct
  REGION 1..8: note = Synthetic peptide
MGAQFAKK

SEQ ID NO: 89 - protein, 8 aa, synthetic construct
  REGION 1..8: note = Synthetic peptide
MGAQSSKK

SEQ ID NO: 90 - protein, 8 aa, synthetic construct
  REGION 1..8: note = Synthetic peptide
MGAQSAKK

SEQ ID NO: 91 - protein, 8 aa, synthetic construct
  REGION 1..8: note = Synthetic peptide
MGAQQSKK

SEQ ID NO: 92 - protein, 8 aa, synthetic construct
  REGION 1..8: note = Synthetic peptide
MGAQQAKK

SEQ ID NO: 93 - protein, 8 aa, synthetic construct
  REGION 1..8: note = Synthetic peptide
MGSKLSKK

SEQ ID NO: 94 - protein, 8 aa, synthetic construct
  REGION 1..8: note = Synthetic peptide
MGSKLAKK

SEQ ID NO: 95 - protein, 8 aa, synthetic construct
  REGION 1..8: note = Synthetic peptide
MGSKFSKK

SEQ ID NO: 96 - protein, 8 aa, synthetic construct
  REGION 1..8: note = Synthetic peptide
MGSKFAKK

SEQ ID NO: 97 - protein, 8 aa, synthetic construct
  REGION 1..8: note = Synthetic peptide
MGSKSSKK

SEQ ID NO: 98 - protein, 8 aa, synthetic construct
  REGION 1..8: note = Synthetic peptide
MGSKSAKK

SEQ ID NO: 99 - protein, 8 aa, synthetic construct
  REGION 1..8: note = Synthetic peptide
MGSKQSKK

SEQ ID NO: 100 - protein, 8 aa, synthetic construct
  REGION 1..8: note = Synthetic peptide
MGSKQAKK

SEQ ID NO: 101 - protein, 8 aa, synthetic construct
  REGION 1..8: note = Synthetic peptide
MGSQLSKK

SEQ ID NO: 102 - protein, 8 aa, synthetic construct
  REGION 1..8: note = Synthetic peptide
MGSQLAKK

SEQ ID NO: 103 - protein, 8 aa, synthetic construct
  REGION 1..8: note = Synthetic peptide
MGSQFSKK

SEQ ID NO: 104 - protein, 8 aa, synthetic construct
  REGION 1..8: note = Synthetic peptide
MGSQFAKK

SEQ ID NO: 105 - protein, 8 aa, synthetic construct
  REGION 1..8: note = Synthetic peptide
MGSQSSKK

SEQ ID NO: 106 - protein, 8 aa, synthetic construct
  REGION 1..8: note = Synthetic peptide
MGSQSAKK

SEQ ID NO: 107 - protein, 8 aa, synthetic construct
  REGION 1..8: note = Synthetic peptide
MGSQQSKK

SEQ ID NO: 108 - protein, 8 aa, synthetic construct
  REGION 1..8: note = Synthetic peptide
MGSQQAKK

SEQ ID NO: 109 - protein, 7 aa, synthetic construct
  REGION 1..7: note = Synthetic peptide
MGGKLAK

SEQ ID NO: 110 - protein, 7 aa, synthetic construct
  REGION 1..7: note = Synthetic peptide
MGGKFSK

SEQ ID NO: 111 - protein, 7 aa, synthetic construct
  REGION 1..7: note = Synthetic peptide
MGGKFAK

SEQ ID NO: 112 - protein, 7 aa, synthetic construct
  REGION 1..7: note = Synthetic peptide
MGGKSSK

SEQ ID NO: 113 - protein, 7 aa, synthetic construct
  REGION 1..7: note = Synthetic peptide
MGGKSAK

SEQ ID NO: 114 - protein, 7 aa, synthetic construct
  REGION 1..7: note = Synthetic peptide
MGGKQSK

SEQ ID NO: 115 - protein, 7 aa, synthetic construct
  REGION 1..7: note = Synthetic peptide
MGGKQAK

SEQ ID NO: 116 - protein, 7 aa, synthetic construct
  REGION 1..7: note = Synthetic peptide
MGGQLSK

SEQ ID NO: 117 - protein, 7 aa, synthetic construct
  REGION 1..7: note = Synthetic peptide
MGGQLAK

SEQ ID NO: 118 - protein, 7 aa, synthetic construct
  REGION 1..7: note = Synthetic peptide
MGGQFSK

SEQ ID NO: 119 - protein, 7 aa, synthetic construct
  REGION 1..7: note = Synthetic peptide
MGGQFAK

SEQ ID NO: 120 - protein, 7 aa, synthetic construct
  REGION 1..7: note = Synthetic peptide
MGGQSSK

SEQ ID NO: 121 - protein, 7 aa, synthetic construct
  REGION 1..7: note = Synthetic peptide
MGGQSAK

SEQ ID NO: 122 - protein, 7 aa, synthetic construct
  REGION 1..7: note = Synthetic peptide
MGGQQSK

SEQ ID NO: 123 - protein, 7 aa, synthetic construct
  REGION 1..7: note = Synthetic peptide
MGGQQAK

SEQ ID NO: 124 - protein, 7 aa, synthetic construct
  REGION 1..7: note = Synthetic peptide
MGAKLSK

SEQ ID NO: 125 - protein, 7 aa, synthetic construct
  REGION 1..7: note = Synthetic peptide
MGAKLAK

SEQ ID NO: 126 - protein, 7 aa, synthetic construct
  REGION 1..7: note = Synthetic peptide
MGAKFSK

SEQ ID NO: 127 - protein, 7 aa, synthetic construct
  REGION 1..7: note = Synthetic peptide
MGAKFAK

SEQ ID NO: 128 - protein, 7 aa, synthetic construct
  REGION 1..7: note = Synthetic peptide
MGAKSSK

SEQ ID NO: 129 - protein, 7 aa, synthetic construct
  REGION 1..7: note = Synthetic peptide
MGAKSAK

SEQ ID NO: 130 - protein, 7 aa, synthetic construct
  REGION 1..7: note = Synthetic peptide
MGAKQSK

SEQ ID NO: 131 - protein, 7 aa, synthetic construct
  REGION 1..7: note = Synthetic peptide
MGAKQAK

SEQ ID NO: 132 - protein, 7 aa, synthetic construct
  REGION 1..7: note = Synthetic peptide
MGAQLSK

SEQ ID NO: 133 - protein, 7 aa, synthetic construct
  REGION 1..7: note = Synthetic peptide
MGAQLAK

SEQ ID NO: 134 - protein, 7 aa, synthetic construct
  REGION 1..7: note = Synthetic peptide
MGAQFSK

SEQ ID NO: 135 - protein, 7 aa, synthetic construct
  REGION 1..7: note = Synthetic peptide
MGAQFAK

SEQ ID NO: 136 - protein, 7 aa, synthetic construct
  REGION 1..7: note = Synthetic peptide
MGAQSSK

SEQ ID NO: 137 - protein, 7 aa, synthetic construct
  REGION 1..7: note = Synthetic peptide
MGAQSAK

SEQ ID NO: 138 - protein, 7 aa, synthetic construct
  REGION 1..7: note = Synthetic peptide
MGAQQSK

SEQ ID NO: 139 - protein, 7 aa, synthetic construct
  REGION 1..7: note = Synthetic peptide
MGAQQAK

SEQ ID NO: 140 - protein, 7 aa, synthetic construct
  REGION 1..7: note = Synthetic peptide
MGSKLSK

SEQ ID NO: 141 - protein, 7 aa, synthetic construct
  REGION 1..7: note = Synthetic peptide
MGSKLAK

SEQ ID NO: 142 - protein, 7 aa, synthetic construct
  REGION 1..7: note = Synthetic peptide
MGSKFSK

SEQ ID NO: 143 - protein, 7 aa, synthetic construct
  REGION 1..7: note = Synthetic peptide
MGSKFAK

SEQ ID NO: 144 - protein, 7 aa, synthetic construct
  REGION 1..7: note = Synthetic peptide
MGSKSSK

SEQ ID NO: 145 - protein, 7 aa, synthetic construct
  REGION 1..7: note = Synthetic peptide
MGSKSAK

SEQ ID NO: 146 - protein, 7 aa, synthetic construct
  REGION 1..7: note = Synthetic peptide
MGSKQSK

SEQ ID NO: 147 - protein, 7 aa, synthetic construct
  REGION 1..7: note = Synthetic peptide
MGSKQAK

SEQ ID NO: 148 - protein, 7 aa, synthetic construct
  REGION 1..7: note = Synthetic peptide
MGSQLSK

SEQ ID NO: 149 - protein, 7 aa, synthetic construct
  REGION 1..7: note = Synthetic peptide
MGSQLAK

SEQ ID NO: 150 - protein, 7 aa, synthetic construct
  REGION 1..7: note = Synthetic peptide
MGSQFSK

SEQ ID NO: 151 - protein, 7 aa, synthetic construct
  REGION 1..7: note = Synthetic peptide
MGSQFAK

SEQ ID NO: 152 - protein, 7 aa, synthetic construct
  REGION 1..7: note = Synthetic peptide
MGSQSSK

SEQ ID NO: 153 - protein, 7 aa, synthetic construct
  REGION 1..7: note = Synthetic peptide
MGSQSAK

SEQ ID NO: 154 - protein, 7 aa, synthetic construct
  REGION 1..7: note = Synthetic peptide
MGSQQSK

SEQ ID NO: 155 - protein, 7 aa, synthetic construct
  REGION 1..7: note = Synthetic peptide
MGSQQAK

SEQ ID NO: 156 - protein, 9 aa, synthetic construct
  REGION 1..9: note = Synthetic peptide
MGAKLSKKK

SEQ ID NO: 157 - protein, 167 aa, synthetic construct
  REGION 1..167: note = Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 158 - protein, 167 aa, synthetic construct
  REGION 1..167: note = Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIY

SEQ ID NO: 159 - protein, 296 aa, synthetic construct
  REGION 1..296: note = Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGGSGGSGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 160 - protein, 296 aa, synthetic construct
  REGION 1..296: note = Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIYGSGGSGGSGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIY

SEQ ID NO: 161 - RNA, 680 nt, synthetic construct
  misc_feature 1..680: note = Description of Artificial Sequence: Synthetic polynucleotide
  source 1..680: mol_type = other RNA
atgaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatccatcgataagcttcatcgaaacatgaggatcacccatatctgcagtcgacatcgaaacatgaggatcacccatgtctgcagtcgacatcgaaacatgaggatcacccatgtctgcagtcgacatcgaaacatgaggatcacccatgtctgcagtcgacatcgaaa

SEQ ID NO: 162 - protein, 9 aa, synthetic construct
  REGION 1..9: note = Synthetic peptide
  VARIANT 3: note = Any Naturally Occuring Amino Acid
MGXKLSKKK

SEQ ID NO: 163 - skipped

SEQ ID NO: 164 - protein, 8 aa, synthetic construct
  REGION 1..8: note = Synthetic peptide
  VARIANT 3: note = Gly, Ala, or Ser
  VARIANT 4: note = Lys or Gln
  VARIANT 5: note = Leu, Phe, Ser, or Gln
  VARIANT 6: note = Ser or Ala
  VARIANT 6: note = Ser or Ala, See specification as filed for detailed description of substitutions and preferred embodiments
MGXXXXKK

SEQ ID NO: 165 - DNA, 20 bp, synthetic construct
  misc_feature 1..20: note = Description of Artificial Sequence: Synthetic primer
  source 1..20: mol_type = other DNA
tggaggtgctcaaagagttg

SEQ ID NO: 166 - DNA, 17 bp, synthetic construct
  misc_feature 1..17: note = Description of Artificial Sequence: Synthetic primer
  source 1..17: mol_type = other DNA
ttgggcgtgcacttgat

SEQ ID NO: 167 - DNA, 13 bp, synthetic construct
  misc_feature 1..13: note = Description of Artificial Sequence: Synthetic probe
  source 1..13: mol_type = other DNA
gggcattggcttc

SEQ ID NO: 168 - protein, 60 aa, synthetic construct
  REGION 1..60: note = Description of Artificial Sequence: Synthetic polypeptide
MGGKLSKKKKGYNVNDEKAKEKDKKAEGAASAGGGGSDYKDDDDKGGGGSVSKGEELFTG

SEQ ID NO: 169 - protein, 60 aa, synthetic construct
  REGION 1..60: note = Description of Artificial Sequence: Synthetic polypeptide
MAGKLSKKKKGYNVNDEKAKEKDKKAEGAASAGGGGSDYKDDDDKGGGGSVSKGEELFTG

SEQ ID NO: 170 - protein, 60 aa, synthetic construct
  REGION 1..60: note = Description of Artificial Sequence: Synthetic polypeptide
MGAKLSKKKKGYNVNDEKAKEKDKKAEGAASAGGGGSDYKDDDDKGGGGSVSKGEELFTG

SEQ ID NO: 171 - protein, 60 aa, synthetic construct
  REGION 1..60: note = Description of Artificial Sequence: Synthetic polypeptide
MAAKLSKKKKGYNVNDEKAKEKDKKAEGAASAGGGGSDYKDDDDKGGGGSVSKGEELFTG

SEQ ID NO: 172 - protein, 57 aa, synthetic construct
  REGION 1..57: note = Description of Artificial Sequence: Synthetic polypeptide
MGGKLSKKKKGYNVNDEKAKEKDKKAESAGGGGSDYKDDDDKGGGGSVSKGEELFTG

SEQ ID NO: 173 - protein, 54 aa, synthetic construct
  REGION 1..54: note = Description of Artificial Sequence: Synthetic polypeptide
MGGKLSKKKKGYNVNDEKAKEKDKSAGGGGSDYKDDDDKGGGGSVSKGEELFTG

SEQ ID NO: 174 - protein, 51 aa, synthetic construct
  REGION 1..51: note = Description of Artificial Sequence: Synthetic polypeptide
MGGKLSKKKKGYNVNDEKAKESAGGGGSDYKDDDDKGGGGSVSKGEELFTG

SEQ ID NO: 175 - protein, 48 aa, synthetic construct
  REGION 1..48: note = Description of Artificial Sequence: Synthetic polypeptide
MGGKLSKKKKGYNVNDEKSAGGGGSDYKDDDDKGGGGSVSKGEELFTG

SEQ ID NO: 176 - protein, 45 aa, synthetic construct
  REGION 1..45: note = Description of Artificial Sequence: Synthetic polypeptide
MGGKLSKKKKGYNVNSAGGGGSDYKDDDDKGGGGSVSKGEELFTG

SEQ ID NO: 177 - protein, 42 aa, synthetic construct
  REGION 1..42: note = Description of Artificial Sequence: Synthetic polypeptide
MGGKLSKKKKGYSAGGGGSDYKDDDDKGGGGSVSKGEELFTG

SEQ ID NO: 178 - protein, 39 aa, synthetic construct
  REGION 1..39: note = Description of Artificial Sequence: Synthetic polypeptide
MGGKLSKKKSAGGGGSDYKDDDDKGGGGSVSKGEELFTG

SEQ ID NO: 179 - protein, 36 aa, synthetic construct
  REGION 1..36: note = Description of Artificial Sequence: Synthetic polypeptide
MGGKLSSAGGGGSDYKDDDDKGGGGSVSKGEELFTG

SEQ ID NO: 180 - protein, 33 aa, synthetic construct
  REGION 1..33: note = Description of Artificial Sequence: Synthetic polypeptide
MGGSAGGGGSDYKDDDDKGGGGSVSKGEELFTG

SEQ ID NO: 181 - protein, 54 aa, synthetic construct
  REGION 1..54: note = Description of Artificial Sequence: Synthetic polypeptide
MGGKLSKKKKGYNVNDEKAKEKDKKAEGAASAGGGGSDYKDDDDKGGGGSVSKG

SEQ ID NO: 182 - protein, 36 aa, synthetic construct
  REGION 1..36: note = Description of Artificial Sequence: Synthetic polypeptide
MGGKLSKKKKGYSAGGGGSDYKDDDDKGGGGSVSKG

SEQ ID NO: 183 - protein, 35 aa, synthetic construct
  REGION 1..35: note = Description of Artificial Sequence: Synthetic polypeptide
MGGKLSKKKKGSAGGGGSDYKDDDDKGGGGSVSKG

SEQ ID NO: 184 - protein, 34 aa, synthetic construct
  REGION 1..34: note = Description of Artificial Sequence: Synthetic polypeptide
MGGKLSKKKKSAGGGGSDYKDDDDKGGGGSVSKG

SEQ ID NO: 185 - protein, 33 aa, synthetic construct
  REGION 1..33: note = Description of Artificial Sequence: Synthetic polypeptide
MGGKLSKKKSAGGGGSDYKDDDDKGGGGSVSKG

SEQ ID NO: 186 - protein, 32 aa, synthetic construct
  REGION 1..32: note = Description of Artificial Sequence: Synthetic polypeptide
MGGKLSKKSAGGGGSDYKDDDDKGGGGSVSKG

SEQ ID NO: 187 - protein, 31 aa, synthetic construct
  REGION 1..31: note = Description of Artificial Sequence: Synthetic polypeptide
MGGKLSKSAGGGGSDYKDDDDKGGGGSVSKG

SEQ ID NO: 188 - protein, 30 aa, synthetic construct
  REGION 1..30: note = Description of Artificial Sequence: Synthetic polypeptide
MGGKLSSAGGGGSDYKDDDDKGGGGSVSKG

SEQ ID NO: 189 - protein, 30 aa, synthetic construct
  REGION 1..30: note = Description of Artificial Sequence: Synthetic polypeptide
MGGKLDKKKKGYNVNDEKAKEKDKKAEGAA

SEQ ID NO: 190 - protein, 30 aa, synthetic construct
  REGION 1..30: note = Description of Artificial Sequence: Synthetic polypeptide
MGGKLAKKKKGYNVNDEKAKEKDKKAEGAA

SEQ ID NO: 191 - protein, 30 aa, synthetic construct
  REGION 1..30: note = Description of Artificial Sequence: Synthetic polypeptide
MGGKQSKKKKGYNVNDEKAKEKDKKAEGAA

SEQ ID NO: 192 - protein, 30 aa, synthetic construct
  REGION 1..30: note = Description of Artificial Sequence: Synthetic polypeptide
MGAKKKKKRFSFKKSFKLSGFSFKKNKKEA

SEQ ID NO: 193 - protein, 30 aa, synthetic construct
  REGION 1..30: note = Description of Artificial Sequence: Synthetic polypeptide
MAAKKKKKRFSFKKSFKLSGFSFKKNKKEA

SEQ ID NO: 194 - protein, 30 aa, synthetic construct
  REGION 1..30: note = Description of Artificial Sequence: Synthetic polypeptide
MGAKKSKKRFSFKKSFKLSGFSFKKNKKEA

SEQ ID NO: 195 - protein, 30 aa, synthetic construct
  REGION 1..30: note = Description of Artificial Sequence: Synthetic polypeptide
MGAKKAKKRFSFKKPFKLSGFSFKKNKKEA

SEQ ID NO: 196 - protein, 153 aa, synthetic construct
  REGION 1..153: note = Description of Artificial Sequence: Synthetic polypeptide
MGGKLSKKKKSAGGSGGSTSGSGDYKDDDDKGSGFEMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 197 - protein, 4 aa, synthetic construct
  REGION 1..4: note = Peptide Linker
GGGG

SEQ ID NO: 198 - protein, 7 aa, synthetic construct
  REGION 1..7: note = Peptide Linker
SGGSGGS

SEQ ID NO: 199 - protein, 15 aa, synthetic construct
  REGION 1..15: note = Peptide Linker
GGSGGSGGSGGSGGG

SEQ ID NO: 200 - protein, 16 aa, synthetic construct
  REGION 1..16: note = Peptide Linker
GGSGGSGGGGSGGGGS

SEQ ID NO: 201 - protein, 18 aa, synthetic construct
  REGION 1..18: note = Peptide Linker
GGSGGSGGSGGSGGSGGS

SEQ ID NO: 202 - protein, 15 aa, synthetic construct
  REGION 1..15: note = Peptide Linker
GGGGSGGGGSGGGGS

SEQ ID NO: 203 - protein, 5 aa, synthetic construct
  REGION 1..5: note = Peptide Linker
  VARIANT 5: note = Xaa can be any integer from 1-100
GGGSX

SEQ ID NO: 204 - protein, 10 aa, synthetic construct
  REGION 1..10: note = Peptide Linker
  VARIANT 4: note = Wherein Xaa can be an integer from 1-100
  VARIANT 10: note = Wherein Xaa can be an integer from 1-100
GGSXGGGGSX

SEQ ID NO: 205 - protein, 4 aa, synthetic construct
  REGION 1..4: note = Effector Domain
KKKK

SEQ ID NO: 206 - protein, 5 aa, synthetic construct
  REGION 1..5: note = Effector Domain
KKKKK

SEQ ID NO: 207 - protein, 4 aa, synthetic construct
  REGION 1..4: note = Effector Domain
RRRR

SEQ ID NO: 208 - protein, 5 aa, synthetic construct
  REGION 1..5: note = Effector Domain
RRRRR

SEQ ID NO: 209 - protein, 4 aa, synthetic construct
  REGION 1..4: note = Effector Domain
  VARIANT 1..4: note = Lys can be Arg
KKKK

SEQ ID NO: 210 - protein, 5 aa, synthetic construct
  REGION 1..5: note = Effector Domain
  VARIANT 1..5: note = Lys can be Arg
KKKKK

SEQ ID NO: 211 - protein, 7 aa, synthetic construct
  REGION 1..7: note = Scaffold Protein
GGKLSKK

SEQ ID NO: 212 - protein, 7 aa, synthetic construct
  REGION 1..7: note = Scaffold Protein
GAKLSKK

SEQ ID NO: 213 - protein, 7 aa, synthetic construct
  REGION 1..7: note = Scaffold Protein
GGKQSKK

SEQ ID NO: 214 - protein, 7 aa, synthetic construct
  REGION 1..7: note = Scaffold Protein
GGKLAKK

SEQ ID NO: 215 - protein, 6 aa, synthetic construct
  REGION 1..6: note = Scaffold Protein
GGKLSK

SEQ ID NO: 216 - protein, 6 aa, synthetic construct
  REGION 1..6: note = Scaffold Protein
GAKLSK

SEQ ID NO: 217 - protein, 6 aa, synthetic construct
  REGION 1..6: note = Scaffold Protein
GGKQSK

SEQ ID NO: 218 - protein, 6 aa, synthetic construct
  REGION 1..6: note = Scaffold Protein
GGKLAK

SEQ ID NO: 219 - protein, 4 aa, synthetic construct
  REGION 1..4: note = Scaffold Protein
KKKG

SEQ ID NO: 220 - protein, 5 aa, synthetic construct
  REGION 1..5: note = Scaffold Protein
KKKGY

SEQ ID NO: 221 - protein, 6 aa, synthetic construct
  REGION 1..6: note = Scaffold Protein
KKKGYN

SEQ ID NO: 222 - protein, 7 aa, synthetic construct
  REGION 1..7: note = Scaffold Protein
KKKGYNV

SEQ ID NO: 223 - protein, 8 aa, synthetic construct
  REGION 1..8: note = Scaffold Protein
KKKGYNVN

SEQ ID NO: 224 - protein, 6 aa, synthetic construct
  REGION 1..6: note = Scaffold Protein
KKKGYS

SEQ ID NO: 225 - protein, 6 aa, synthetic construct
  REGION 1..6: note = Scaffold Protein
KKKGYG

SEQ ID NO: 226 - protein, 7 aa, synthetic construct
  REGION 1..7: note = Scaffold Protein
KKKGYGG

SEQ ID NO: 227 - protein, 5 aa, synthetic construct
  REGION 1..5: note = Scaffold Protein
KKKGS

SEQ ID NO: 228 - protein, 6 aa, synthetic construct
  REGION 1..6: note = Scaffold Protein
KKKGSG

SEQ ID NO: 229 - protein, 7 aa, synthetic construct
  REGION 1..7: note = Scaffold Protein
KKKGSGS

SEQ ID NO: 230 - protein, 4 aa, synthetic construct
  REGION 1..4: note = Scaffold Protein
KKKS

SEQ ID NO: 231 - protein, 5 aa, synthetic construct
  REGION 1..5: note = Scaffold Protein
KKKSG

SEQ ID NO: 232 - protein, 6 aa, synthetic construct
  REGION 1..6: note = Scaffold Protein
KKKSGG

SEQ ID NO: 233 - protein, 7 aa, synthetic construct
  REGION 1..7: note = Scaffold Protein
KKKSGGS

SEQ ID NO: 234 - protein, 8 aa, synthetic construct
  REGION 1..8: note = Scaffold Protein
KKKSGGSG

SEQ ID NO: 235 - protein, 8 aa, synthetic construct
  REGION 1..8: note = Scaffold Protein
KKSGGSGG

SEQ ID NO: 236 - protein, 10 aa, synthetic construct
  REGION 1..10: note = Scaffold Protein
KKKSGGSGGS

SEQ ID NO: 237 - protein, 8 aa, synthetic construct
  REGION 1..8: note = Scaffold Protein
KRFSFKKS

SEQ ID NO: 238 - protein, 8 aa, synthetic construct
  REGION 1..8: note = Scaffold Protein
GGKLSKKK

SEQ ID NO: 239 - protein, 8 aa, synthetic construct
  REGION 1..8: note = Scaffold Protein
GGKLSKKS

SEQ ID NO: 240 - protein, 8 aa, synthetic construct
  REGION 1..8: note = Scaffold Protein
GAKLSKKK

SEQ ID NO: 241 - protein, 8 aa, synthetic construct
  REGION 1..8: note = Scaffold Protein
GAKLSKKS

SEQ ID NO: 242 - protein, 8 aa, synthetic construct
  REGION 1..8: note = Scaffold Protein
GGKQSKKK

SEQ ID NO: 243 - protein, 8 aa, synthetic construct
  REGION 1..8: note = Scaffold Protein
GGKQSKKS

SEQ ID NO: 244 - protein, 8 aa, synthetic construct
  REGION 1..8: note = Scaffold Protein
GGKLAKKK

SEQ ID NO: 245 - protein, 8 aa, synthetic construct
  REGION 1..8: note = Scaffold Protein
GGKLAKKS

SEQ ID NO: 246 - protein, 14 aa, synthetic construct
  REGION 1..14: note = Scaffold Protein
GGKLSKKKKGYNVN

SEQ ID NO: 247 - protein, 14 aa, synthetic construct
  REGION 1..14: note = Scaffold Protein
GAKLSKKKKGYNVN

SEQ ID NO: 248 - protein, 14 aa, synthetic construct
  REGION 1..14: note = Scaffold Protein
GGKQSKKKKGYNVN

SEQ ID NO: 249 - protein, 14 aa, synthetic construct
  REGION 1..14: note = Scaffold Protein
GGKLAKKKKGYNVN

SEQ ID NO: 250 - protein, 14 aa, synthetic construct
  REGION 1..14: note = Scaffold Protein
GGKLSKKKKGYSGG

SEQ ID NO: 251 - protein, 14 aa, synthetic construct
  REGION 1..14: note = Scaffold Protein
GGKLSKKKKGSGGS

SEQ ID NO: 252 - protein, 14 aa, synthetic construct
  REGION 1..14: note = Scaffold Protein
GGKLSKKKKSGGSG

SEQ ID NO: 253 - protein, 14 aa, synthetic construct
  REGION 1..14: note = Scaffold Protein
GGKLSKKKSGGSGG

SEQ ID NO: 254 - protein, 14 aa, synthetic construct
  REGION 1..14: note = Scaffold Protein
GGKLSKKSGGSGGS

SEQ ID NO: 255 - protein, 14 aa, synthetic construct
  REGION 1..14: note = Scaffold Protein
GGKLSKSGGSGGSV

SEQ ID NO: 256 - protein, 14 aa, synthetic construct
  REGION 1..14: note = Scaffold Protein
GAKKSKKRFSFKKS

SEQ ID NO: 257 - protein, 29 aa, synthetic construct
  REGION 1..29: note = Scaffold Protein
GGKLSKKKKGYNVNDEKAKEKDKKAEGAA

SEQ ID NO: 258 - protein, 28 aa, synthetic construct
  REGION 1..28: note = Scaffold Protein
GGKLSKKKKGYNVNDEKAKEKDKKAEGA

SEQ ID NO: 259 - protein, 27 aa, synthetic construct
  REGION 1..27: note = Scaffold Protein
GGKLSKKKKGYNVNDEKAKEKDKKAEG

SEQ ID NO: 260 - protein, 26 aa, synthetic construct
  REGION 1..26: note = Scaffold Protein
GGKLSKKKKGYNVNDEKAKEKDKKAE

SEQ ID NO: 261 - protein, 25 aa, synthetic construct
  REGION 1..25: note = Scaffold Protein
GGKLSKKKKGYNVNDEKAKEKDKKA

SEQ ID NO: 262 - protein, 24 aa, synthetic construct
  REGION 1..24: note = Scaffold Protein
GGKLSKKKKGYNVNDEKAKEKDKK

SEQ ID NO: 263 - protein, 23 aa, synthetic construct
  REGION 1..23: note = Scaffold Protein
GGKLSKKKKGYNVNDEKAKEKDK

SEQ ID NO: 264 - protein, 22 aa, synthetic construct
  REGION 1..22: note = Scaffold Protein
GGKLSKKKKGYNVNDEKAKEKD

SEQ ID NO: 265 - protein, 21 aa, synthetic construct
  REGION 1..21: note = Scaffold Protein
GGKLSKKKKGYNVNDEKAKEK

SEQ ID NO: 266 - protein, 20 aa, synthetic construct
  REGION 1..20: note = Scaffold Protein
GGKLSKKKKGYNVNDEKAKE

SEQ ID NO: 267 - protein, 19 aa, synthetic construct
  REGION 1..19: note = Scaffold Protein
GGKLSKKKKGYNVNDEKAK

SEQ ID NO: 268 - protein, 18 aa, synthetic construct
  REGION 1..18: note = Scaffold Protein
GGKLSKKKKGYNVNDEKA

SEQ ID NO: 269 - protein, 17 aa, synthetic construct
  REGION 1..17: note = Scaffold Protein
GGKLSKKKKGYNVNDEK

SEQ ID NO: 270 - protein, 16 aa, synthetic construct
  REGION 1..16: note = Scaffold Protein
GGKLSKKKKGYNVNDE

SEQ ID NO: 271 - protein, 15 aa, synthetic construct
  REGION 1..15: note = Scaffold Protein
GGKLSKKKKGYNVND

SEQ ID NO: 272 - protein, 13 aa, synthetic construct
  REGION 1..13: note = Scaffold Protein
GGKLSKKKKGYNV

SEQ ID NO: 273 - protein, 12 aa, synthetic construct
  REGION 1..12: note = Scaffold Protein
GGKLSKKKKGYN

SEQ ID NO: 274 - protein, 11 aa, synthetic construct
  REGION 1..11: note = Scaffold Protein
GGKLSKKKKGY

SEQ ID NO: 275 - protein, 10 aa, synthetic construct
  REGION 1..10: note = Scaffold Protein
GGKLSKKKKG

SEQ ID NO: 276 - protein, 9 aa, synthetic construct
  REGION 1..9: note = Scaffold Protein
GGKLSKKKK

SEQ ID NO: 277 - protein, 28 aa, synthetic construct
  REGION 1..28: note = Scaffold Protein
GAKKSKKRFSFKKSFKLSGFSFKKNKKE

SEQ ID NO: 278 - protein, 27 aa, synthetic construct
  REGION 1..27: note = Scaffold Protein
GAKKSKKRFSFKKSFKLSGFSFKKNKK

SEQ ID NO: 279 - protein, 26 aa, synthetic construct
  REGION 1..26: note = Scaffold Protein
GAKKSKKRFSFKKSFKLSGFSFKKNK

SEQ ID NO: 280 - protein, 25 aa, synthetic construct
  REGION 1..25: note = Scaffold Protein
GAKKSKKRFSFKKSFKLSGFSFKKN

SEQ ID NO: 281 - protein, 24 aa, synthetic construct
  REGION 1..24: note = Scaffold Protein
GAKKSKKRFSFKKSFKLSGFSFKK

SEQ ID NO: 282 - protein, 23 aa, synthetic construct
  REGION 1..23: note = Scaffold Protein
GAKKSKKRFSFKKSFKLSGFSFK

SEQ ID NO: 283 - protein, 22 aa, synthetic construct
  REGION 1..22: note = Scaffold Protein
GAKKSKKRFSFKKSFKLSGFSF

SEQ ID NO: 284 - protein, 21 aa, synthetic construct
  REGION 1..21: note = Scaffold Protein
GAKKSKKRFSFKKSFKLSGFS

SEQ ID NO: 285 - protein, 20 aa, synthetic construct
  REGION 1..20: note = Scaffold Protein
GAKKSKKRFSFKKSFKLSGF

SEQ ID NO: 286 - protein, 19 aa, synthetic construct
  REGION 1..19: note = Scaffold Protein
GAKKSKKRFSFKKSFKLSG

SEQ ID NO: 287 - protein, 18 aa, synthetic construct
  REGION 1..18: note = Scaffold Protein
GAKKSKKRFSFKKSFKLS

SEQ ID NO: 288 - protein, 17 aa, synthetic construct
  REGION 1..17: note = Scaffold Protein
GAKKSKKRFSFKKSFKL

SEQ ID NO: 289 - protein, 16 aa, synthetic construct
  REGION 1..16: note = Scaffold Protein
GAKKSKKRFSFKKSFK

SEQ ID NO: 290 - protein, 15 aa, synthetic construct
  REGION 1..15: note = Scaffold Protein
GAKKSKKRFSFKKSF

SEQ ID NO: 291 - protein, 14 aa, synthetic construct
  REGION 1..14: note = Scaffold Protein
GAKKSKKRFSFKKS

SEQ ID NO: 292 - protein, 13 aa, synthetic construct
  REGION 1..13: note = Scaffold Protein
GAKKSKKRFSFKK

SEQ ID NO: 293 - protein, 12 aa, synthetic construct
  REGION 1..12: note = Scaffold Protein
GAKKSKKRFSFK

SEQ ID NO: 294 - protein, 11 aa, synthetic construct
  REGION 1..11: note = Scaffold Protein
GAKKSKKRFSF

SEQ ID NO: 295 - protein, 10 aa, synthetic construct
  REGION 1..10: note = Scaffold Protein
GAKKSKKRFS

SEQ ID NO: 296 - protein, 9 aa, synthetic construct
  REGION 1..9: note = Scaffold Protein
GAKKSKKRF

SEQ ID NO: 297 - protein, 8 aa, synthetic construct
  REGION 1..8: note = Scaffold Protein
GAKKSKKR

SEQ ID NO: 298 - protein, 7 aa, synthetic construct
  REGION 1..7: note = Scaffold Protein
GAKKSKK

SEQ ID NO: 299 - protein, 28 aa, synthetic construct
  REGION 1..28: note = Scaffold Protein
GAKKAKKRFSFKKSFKLSGFSFKKNKKE

SEQ ID NO: 300 - protein, 29 aa, synthetic construct
  REGION 1..29: note = Scaffold Protein
GAKKSKKRFSFKKSFKLSGFSFKKNKKEA

SEQ ID NO: 301 - protein, 29 aa, synthetic construct
  REGION 1..29: note = Scaffold Protein
GAKKAKKRFSFKKSFKLSGFSFKKNKKEA

SEQ ID NO: 302 - protein, 28 aa, synthetic construct
  REGION 1..28: note = Scaffold Protein
GAQESKKKKKKRFSFKKSFKLSGFSFKK

SEQ ID NO: 303 - protein, 27 aa, synthetic construct
  REGION 1..27: note = Scaffold Protein
GAQESKKKKKKRFSFKKSFKLSGFSFK

SEQ ID NO: 304 - protein, 26 aa, synthetic construct
  REGION 1..26: note = Scaffold Protein
GAQESKKKKKKRFSFKKSFKLSGFSF

SEQ ID NO: 305 - protein, 25 aa, synthetic construct
  REGION 1..25: note = Scaffold Protein
GAQESKKKKKKRFSFKKSFKLSGFS

SEQ ID NO: 306 - protein, 24 aa, synthetic construct
  REGION 1..24: note = Scaffold Protein
GAQESKKKKKKRFSFKKSFKLSGF

SEQ ID NO: 307 - protein, 23 aa, synthetic construct
  REGION 1..23: note = Scaffold Protein
GAQESKKKKKKRFSFKKSFKLSG

SEQ ID NO: 308 - protein, 22 aa, synthetic construct
  REGION 1..22: note = Scaffold Protein
GAQESKKKKKKRFSFKKSFKLS

SEQ ID NO: 309 - protein, 21 aa, synthetic construct
  REGION 1..21: note = Scaffold Protein
GAQESKKKKKKRFSFKKSFKL

SEQ ID NO: 310 - protein, 20 aa, synthetic construct
  REGION 1..20: note = Scaffold Protein
GAQESKKKKKKRFSFKKSFK

SEQ ID NO: 311 - protein, 19 aa, synthetic construct
  REGION 1..19: note = Scaffold Protein
GAQESKKKKKKRFSFKKSF

SEQ ID NO: 312 - protein, 18 aa, synthetic construct
  REGION 1..18: note = Scaffold Protein
GAQESKKKKKKRFSFKKS

SEQ ID NO: 313 - protein, 17 aa, synthetic construct
  REGION 1..17: note = Scaffold Protein
GAQESKKKKKKRFSFKK

SEQ ID NO: 314 - protein, 16 aa, synthetic construct
  REGION 1..16: note = Scaffold Protein
GAQESKKKKKKRFSFK

SEQ ID NO: 315 - protein, 15 aa, synthetic construct
  REGION 1..15: note = Scaffold Protein
GAQESKKKKKKRFSF

SEQ ID NO: 316 - protein, 14 aa, synthetic construct
  REGION 1..14: note = Scaffold Protein
GAQESKKKKKKRFS

SEQ ID NO: 317 - protein, 13 aa, synthetic construct
  REGION 1..13: note = Scaffold Protein
GAQESKKKKKKRF

SEQ ID NO: 318 - protein, 12 aa, synthetic construct
  REGION 1..12: note = Scaffold Protein
GAQESKKKKKKR

SEQ ID NO: 319 - protein, 11 aa, synthetic construct
  REGION 1..11: note = Scaffold Protein
GAQESKKKKKK

SEQ ID NO: 320 - protein, 10 aa, synthetic construct
  REGION 1..10: note = Scaffold Protein
GAQESKKKKK

SEQ ID NO: 321 - protein, 9 aa, synthetic construct
  REGION 1..9: note = Scaffold Protein
GAQESKKKK

SEQ ID NO: 322 - protein, 8 aa, synthetic construct
  REGION 1..8: note = Scaffold Protein
GAQESKKK

SEQ ID NO: 323 - protein, 7 aa, synthetic construct
  REGION 1..7: note = Scaffold Protein
GAQESKK

SEQ ID NO: 324 - protein, 30 aa, synthetic construct
  REGION 1..30: note = Scaffold Protein
GSQSSKKKKKKFSFKKPFKLSGLSFKRNRK

SEQ ID NO: 325 - protein, 29 aa, synthetic construct
  REGION 1..29: note = Scaffold Protein
GSQSSKKKKKKFSFKKPFKLSGLSFKRNR

SEQ ID NO: 326 - protein, 28 aa, synthetic construct
  REGION 1..28: note = Scaffold Protein
GSQSSKKKKKKFSFKKPFKLSGLSFKRN

SEQ ID NO: 327 - protein, 27 aa, synthetic construct
  REGION 1..27: note = Scaffold Protein
GSQSSKKKKKKFSFKKPFKLSGLSFKR

SEQ ID NO: 328 - protein, 26 aa, synthetic construct
  REGION 1..26: note = Scaffold Protein
GSQSSKKKKKKFSFKKPFKLSGLSFK

SEQ ID NO: 329 - protein, 25 aa, synthetic construct
  REGION 1..25: note = Scaffold Protein
GSQSSKKKKKKFSFKKPFKLSGLSF

SEQ ID NO: 330 - protein, 24 aa, synthetic construct
  REGION 1..24: note = Scaffold Protein
GSQSSKKKKKKFSFKKPFKLSGLS

SEQ ID NO: 331 - protein, 23 aa, synthetic construct
  REGION 1..23: note = Scaffold Protein
GSQSSKKKKKKFSFKKPFKLSGL

SEQ ID NO: 332 - protein, 22 aa, synthetic construct
  REGION 1..22: note = Scaffold Protein
GSQSSKKKKKKFSFKKPFKLSG

SEQ ID NO: 333 - protein, 21 aa, synthetic construct
  REGION 1..21: note = Scaffold Protein
GSQSSKKKKKKFSFKKPFKLS

SEQ ID NO: 334 - protein, 20 aa, synthetic construct
  REGION 1..20: note = Scaffold Protein
GSQSSKKKKKKFSFKKPFKL

SEQ ID NO: 335 - protein, 19 aa, synthetic construct
  REGION 1..19: note = Scaffold Protein
GSQSSKKKKKKFSFKKPFK

SEQ ID NO: 336 - protein, 18 aa, synthetic construct
  REGION 1..18: note = Scaffold Protein
GSQSSKKKKKKFSFKKPF

SEQ ID NO: 337 - protein, 17 aa, synthetic construct
  REGION 1..17: note = Scaffold Protein
GSQSSKKKKKKFSFKKP

SEQ ID NO: 338 - protein, 16 aa, synthetic construct
  REGION 1..16: note = Scaffold Protein
GSQSSKKKKKKFSFKK

SEQ ID NO: 339 - protein, 15 aa, synthetic construct
  REGION 1..15: note = Scaffold Protein
GSQSSKKKKKKFSFK

SEQ ID NO: 340 - protein, 14 aa, synthetic construct
  REGION 1..14: note = Scaffold Protein
GSQSSKKKKKKFSF

SEQ ID NO: 341 - protein, 13 aa, synthetic construct
  REGION 1..13: note = Scaffold Protein
GSQSSKKKKKKFS

SEQ ID NO: 342 - protein, 12 aa, synthetic construct
  REGION 1..12: note = Scaffold Protein
GSQSSKKKKKKF

SEQ ID NO: 343 - protein, 11 aa, synthetic construct
  REGION 1..11: note = Scaffold Protein
GSQSSKKKKKK

SEQ ID NO: 344 - protein, 10 aa, synthetic construct
  REGION 1..10: note = Scaffold Protein
GSQSSKKKKK

SEQ ID NO: 345 - protein, 9 aa, synthetic construct
  REGION 1..9: note = Scaffold Protein
GSQSSKKKK

SEQ ID NO: 346 - protein, 8 aa, synthetic construct
  REGION 1..8: note = Scaffold Protein
GSQSSKKK

SEQ ID NO: 347 - protein, 7 aa, synthetic construct
  REGION 1..7: note = Scaffold Protein
GSQSSKK

SEQ ID NO: 348 - protein, 28 aa, synthetic construct
  REGION 1..28: note = Scaffold Protein
GAKKAKKRFSFKKSFKLSGFSFKKNKKE

SEQ ID NO: 349 - protein, 27 aa, synthetic construct
  REGION 1..27: note = Scaffold Protein
GAKKAKKRFSFKKSFKLSGFSFKKNKK

SEQ ID NO: 350 - protein, 26 aa, synthetic construct
  REGION 1..26: note = Scaffold Protein
GAKKAKKRFSFKKSFKLSGFSFKKNK

SEQ ID NO: 351 - protein, 25 aa, synthetic construct
  REGION 1..25: note = Scaffold Protein
GAKKAKKRFSFKKSFKLSGFSFKKN

SEQ ID NO: 352 - protein, 24 aa, synthetic construct
  REGION 1..24: note = Scaffold Protein
GAKKAKKRFSFKKSFKLSGFSFKK

SEQ ID NO: 353 - protein, 23 aa, synthetic construct
  REGION 1..23: note = Scaffold Protein
GAKKAKKRFSFKKSFKLSGFSFK

SEQ ID NO: 354 - protein, 22 aa, synthetic construct
  REGION 1..22: note = Scaffold Protein
GAKKAKKRFSFKKSFKLSGFSF

SEQ ID NO: 355 - protein, 21 aa, synthetic construct
  REGION 1..21: note = Scaffold Protein
GAKKAKKRFSFKKSFKLSGFS

SEQ ID NO: 356 - protein, 20 aa, synthetic construct
  REGION 1..20: note = Scaffold Protein
GAKKAKKRFSFKKSFKLSGF

SEQ ID NO: 357 - protein, 19 aa, synthetic construct
  REGION 1..19: note = Scaffold Protein
GAKKAKKRFSFKKSFKLSG

SEQ ID NO: 358 - protein, 18 aa, synthetic construct
  REGION 1..18: note = Scaffold Protein
GAKKAKKRFSFKKSFKLS

SEQ ID NO: 359 - protein, 17 aa, synthetic construct
  REGION 1..17: note = Scaffold Protein
GAKKAKKRFSFKKSFKL

SEQ ID NO: 360 - protein, 16 aa, synthetic construct
  REGION 1..16: note = Scaffold Protein
GAKKAKKRFSFKKSFK

SEQ ID NO: 361 - protein, 15 aa, synthetic construct
  REGION 1..15: note = Scaffold Protein
GAKKAKKRFSFKKSF

SEQ ID NO: 362 - protein, 14 aa, synthetic construct
  REGION 1..14: note = Scaffold Protein
GAKKAKKRFSFKKS

SEQ ID NO: 363 - protein, 13 aa, synthetic construct
  REGION 1..13: note = Scaffold Protein
GAKKAKKRFSFKK

SEQ ID NO: 364 - protein, 12 aa, synthetic construct
  REGION 1..12: note = Scaffold Protein
GAKKAKKRFSFK

SEQ ID NO: 365 - protein, 11 aa, synthetic construct
  REGION 1..11: note = Scaffold Protein
GAKKAKKRFSF

SEQ ID NO: 366 - protein, 10 aa, synthetic construct
  REGION 1..10: note = Scaffold Protein
GAKKAKKRFS

SEQ ID NO: 367 - protein, 9 aa, synthetic construct
  REGION 1..9: note = Scaffold Protein
GAKKAKKRF

SEQ ID NO: 368 - protein, 8 aa, synthetic construct
  REGION 1..8: note = Scaffold Protein
GAKKAKKR

SEQ ID NO: 369 - protein, 7 aa, synthetic construct
  REGION 1..7: note = Scaffold Protein
GAKKAKK

SEQ ID NO: 370 - protein, 238 aa, synthetic construct
  REGION 1..238: note = Q8BRU4
MHAEEIYTSLQWDIPTSEASQKCQSPSKCSGAWCVVTMISCVVCMGLLATSIFLGIKFFQVSSLVLEQQERLIQQDTALVNLTQWQRKYTLEYCQALLQRSLHSGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 371 - protein, 238 aa, synthetic construct; identical to SEQ ID NO: 370
  REGION 1..238: note = Q8BRU4

SEQ ID NO: 372 - protein, 237 aa, synthetic construct
  REGION 1..237: note = Q8BRU4-2
MHAEEIYTSLQWDIPTSEASQKCQSPSKCSVFQVSSLVLEQQERLIQQDTALVNLTQWQRKYTLEYCQALLQRSLHSGTDASTGPVLLTSPQMVPQTLDSKETGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 373 - protein, 173 aa, synthetic construct
  REGION 1..173: note = Q8BRU4-3
MHAEEIYTSLQWDIPTSEASQKCQSPSKCSGAWCVVTMISCVVCMGLLATSIFLGIKFFQVSSLVLEQQERLIQQDTALVNLTQWQRKYTLEYCQALLQRSLHSGSDCSPCPHNWIQNGKSCYYVFERWEMWNISKKSCLKEGASLFQIDSKEEMSRWNQWILVLGRWLFSSL

SEQ ID NO: 374 - protein, 264 aa, synthetic construct
  REGION 1..264: note = Q8BRU4-4
MHAEEIYTSLQWDIPTSEASQKCQSPSKCSGAWCVVTMISCVVCMGLLATSIFLGIKFFQVSSLVLEQQERLIQQDTALVNLTQWQRKYTLEYCQALLQRSLHSGTDASTGPVLLTSPQMVPQTLDSKETGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 375 - protein, 148 aa, synthetic construct
  REGION 1..148: note = Q8BRU4-5
MHAEEIYTSLQWDIPTSEASQKCQSPSKCSGAWCVVTMISCVVCMGLLATSIFLGIKFFQVSSLVLEQQERLIQQDTALVNLTQWQRKYTLEYCQALLQRSLHSGCQQKDSDQPARSVDTSKILLSSQISAIAGNILSVRRRHLDPAS

SEQ ID NO: 376 - protein, 8 aa, synthetic construct
  REGION 1..8: note = Scaffold Y
  VARIANT 2: note = X is alanine, or any other amino acid
GXKLSKKK

(19) United States
(12) Patent Application Publication
(10) Pub. No.: US 2026/0216367 A1
(43) Pub. Date: Jul. 30, 2026

(54) EXTRACELLULAR VESICLES TARGETING DENDRITIC CELLS AND USES THEREOF

(71) Applicant: LONZA SALES AG, BASEL (CH)

(72) Inventors: Nikki ROSS, Somerville, MA (US); Dalia BURZYN, Newton, MA (US); Sriram SATHYANARAYANAN, Lexington, MA (US); Raymond J. MONIZ, Brighton, MA (US); Ke XU, Belmont, MA (US); Shelly Ann MARTIN, Stoneham, MA (US); Kevin DOOLEY, Boston, MA (US)

(21) Appl. No.: 19/419,609

(22) Filed: Dec. 15, 2025

Related U.S. Application Data
(63) Continuation of application No. 17/271,176, filed on Aug. 23, 2019, now abandoned.
(60) Provisional application No. 62/870,574, filed on Jul. 3, 2019.

(30) Foreign Application Priority Data
Aug. 24, 2018 (WO) PCT/US2018/048026

(51) Int. Cl. A61K 47/69; A61K 39/00; A61K 47/68; C07K 14/47; C07K 16/28

(52) U.S. Cl. CPC A61K 47/6901; A61K 39/0011; A61K 47/6849; C07K 14/47; C07K 16/2851; A61K 2039/505; A61K 2039/5152; C07K 2317/55; C07K 2317/622; C07K 2319/00

(57) ABSTRACT

The present disclosure relates to modified extracellular vesicles, e.g., exosomes, comprising a targeting moiety, wherein the targeting moiety can specifically bind to markers expressed on distinct immune cells (e.g., dendritic cells). Also provided herein are methods for using the exosomes to treat and/or prevent a range of medical disorders.

### CROSS-REFERENCE TO RELATED APPLICATIONS

This application is a Continuation of U.S. application Ser. No. 17/271,176, which is a US National Stage Entry of PCT application No. PCT/US2019/047937, filed Aug. 23, 2019, which claims the priority benefit of U.S. Provisional Application No. 62/870,574, filed Jul. 3, 2019, and International Application No. PCT/US2018/048026, filed Aug. 24, 2018, all of which are incorporated herein by reference in their entireties.

### REFERENCE TO SEQUENCE LISTING SUBMITTED ELECTRONICALLY VIA EFS-WEB

The application contains a Sequence Listing which has been submitted electronically in XML file, created on Apr. 7, 2026, is named “0132-0272US2.xml”, and is 484,822 bytes in size. The sequence listing contained in this .XML file is part of the specification and is hereby incorporated by reference herein in its entirety.

### FIELD OF DISCLOSURE

The present disclosure relates to modified extracellular vesicles (EVs) (e.g., exosomes) that comprise a targeting moiety, and the use of such EVs to treat and/or prevent a range of medical disorders, such as cancers.

### BACKGROUND OF DISCLOSURE

EVs (e.g., exosomes) are important mediators of intercellular communication. They are also important biomarkers in the diagnosis and prognosis of many diseases, such as cancer. As drug delivery vehicles, EVs (e.g., exosomes) offer many advantages over traditional drug delivery methods (e.g., peptide immunization, DNA vaccines) as a new treatment modality in many therapeutic areas. However, despite its advantages, many EVs (e.g., exosomes) have had limited clinical efficacy. For example, dendritic-cell derived exosomes (DEX) were investigated in a Phase II clinical trial as maintenance immunotherapy after first line chemotherapy in patients with inoperable non-small cell lung cancer (NSCLC). However, the trial was terminated because the primary endpoint (at least 50% of patients with progression-free survival (PFS) at 4 months after chemotherapy cessation) was not reached. Besse, B., et al., Oncoimmunology 5(4):e1071008 (2015).

Accordingly, new and more effective engineered-EVs (e.g., exosomes), particularly those that can specifically target specific immune cells, are necessary to better enable therapeutic use and other applications of EV-based technologies.

### SUMMARY OF DISCLOSURE

Provided here is an extracellular vesicle (EV) comprising an exogenous targeting moiety that specifically binds to a marker for a dendritic cell. In certain aspects, the marker is present only on the dendritic cell. In some aspects, the dendritic cell comprises a plasmacytoid dendritic cell (pDC), a myeloid/conventional dendritic cell 1 (cDC1), a myeloid/conventional dendritic cell 2 (cDC2), or any combination thereof. In certain aspects, the dendritic cell is cDC1.

In some aspects, the marker comprises a C-type lectin domain family 9 member A (CLEC9A) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), or any combination thereof. In certain aspects, the marker comprises an epitope in the Clec9a protein. In certain aspects, the marker comprises a C-type lectin like domain. In some aspects, the C-type lectin like domain comprises the amino acid sequence comprising amino acids 120 to 233 of SEQ ID NO: 1. In some aspects, the marker comprises an extracellular region of Clec9a protein. In certain aspects, the extracellular region of the Clec9a protein comprises the amino acid sequence comprising amino acids 57 to 241 of SEQ ID NO: 1.

In some aspects, the exogenous targeting moiety comprises a microprotein, a designed ankyrin repeat protein (darpin), an anticalin, an adnectin, an aptamer, a peptide mimetic molecule, a natural ligand for a receptor, a camelid nanobody, or any combination thereof. In some aspects, the exogenous targeting moiety comprises a full-length antibody, a single domain antibody, a heavy chain only antibody (VHH), a single chain antibody, a shark heavy chain only antibody (VNAR), an scFv, a Fv, a Fab, a Fab′, a F(ab′)2, or any combination thereof. In certain aspects, the antibody is a single chain antibody.

In some aspects, the EV comprises a scaffold protein linking the exogenous targeting moiety to the EV.

In some aspects, the scaffold protein is a Scaffold X protein. In certain aspects, the Scaffold X protein comprises prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, or any combination thereof. In some aspects, the Scaffold X protein comprises the amino acid sequence set forth as SEQ ID NO: 33. In some aspects, the Scaffold X protein comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 1.

In some aspects, an EV (e.g., exosome) disclosed herein further comprises a Scaffold Y protein. In certain aspects, the Scaffold Y protein comprises myristoylated alanine rich Protein Kinase C substrate (the MARCKS protein), myristoylated alanine rich Protein Kinase C substrate like 1 (the MARCKSL1 protein), brain acid soluble protein 1 (the BASP1 protein), a fragment thereof, and or any combination thereof. In some aspects, the Scaffold Y protein is BASP1 or a fragment thereof.

In some aspects, the Scaffold Y protein comprises an N-terminus domain (ND) and an effector domain (ED), wherein the ND and/or the ED are associated with the luminal surface of the EV. In certain aspects, the ND is associated with the luminal surface of the exosome via myristoylation. In some aspects, the ED is associated with the luminal surface of the exosome by an ionic interaction. In further aspects, the ED comprises (i) a basic amino acid or (ii) two or more basic amino acids in sequence, wherein the basic amino acid is selected from the group consisting of Lys, Arg, His, and any combination thereof. In some aspects, the basic amino acid is (Lys)n, wherein n is an integer between 1 and 10. In some aspects, the ED comprises Lys (K), KK, KKK, KKKK (SEQ ID NO: 205), KKKKK (SEQ ID NO: 206), Arg (R), RR, RRR, RRRR (SEQ ID NO: 207); RRRRR (SEQ ID NO: 208), KR, RK, KKR, KRK, RKK, KRR, RRK, (K/R)(K/R)(K/R)(K/R) (SEQ ID NO: 209), (K/R)(K/R)(K/R)(K/R)(K/R) (SEQ ID NO: 210), or any combination thereof.

In some aspects, the ND comprises the amino acid sequence as set forth in G:X2:X3:X4:X5:X6, wherein G represents Gly; wherein “:” represents a peptide bond, wherein each of the X2 to the X6 is independently an amino acid, and wherein the X6 comprises a basic amino acid. In certain aspects, (i) the X6 is selected from the group consisting of Lys, Arg, and His; (ii) the X5 is selected from the group consisting of Pro, Gly, Ala, and Ser; (iii) the X2 is selected from the group consisting of Pro, Gly, Ala, and Ser; (iv) the X4 is selected from the group consisting of Pro, Gly, Ala, Ser, Val, Ile, Leu, Phe, Trp, Tyr, Gln and Met; or (v) any combination of (i)-(iv).

In some aspects, the ND comprises the amino acid sequence of G:X2:X3:X4:X5:X6, wherein (i) G represents Gly; (ii) “:” represents a peptide bond; (iii) the X2 is an amino acid selected from the group consisting of Pro, Gly, Ala, and Ser; (iv) the X3 is an amino acid; (v) the X4 is an amino acid selected from the group consisting of Pro, Gly, Ala, Ser, Val, Ile, Leu, Phe, Trp, Tyr, Gln and Met; (vi) the X5 is an amino acid selected from the group consisting of Pro, Gly, Ala, and Ser; and (vii) the X6 is an amino acid selected from the group consisting of Lys, Arg, and His. In some aspects, the X3 is selected from the group consisting of Asn, Gln, Ser, Thr, Asp, Glu, Lys, His, and Arg.

In some aspects, the ND and the ED are joined by a linker. In certain aspects, the linker comprises a peptide bond or one or more amino acids.

In some aspects, the ND comprises an amino acid sequence selected from the group consisting of (i) GGKLSKK (SEQ ID NO: 211), (ii) GAKLSKK (SEQ ID NO: 212), (iii) GGKQSKK (SEQ ID NO: 213), (iv) GGKLAKK (SEQ ID NO: 214), and (vi) any combination thereof. In some aspects, the ND comprises an amino acid sequence selected from the group consisting of (i) GGKLSKKK (SEQ ID NO: 238), (ii) GGKLSKKS (SEQ ID NO: 239), (iii) GAKLSKKK (SEQ ID NO: 240), (iv) GAKLSKKS (SEQ ID NO: 241), (v) GGKQSKKK (SEQ ID NO: 242), (vi) GGKQSKKS (SEQ ID NO: 243), (vii) GGKLAKKK (SEQ ID NO: 244), (viii) GGKLAKKS (SEQ ID NO: 245), and (ix) any combination thereof. In certain aspects, the ND comprises the amino acid sequence GGKLSKK (SEQ ID NO: 211).

In some aspects, the scaffold protein is at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 120, at least about 130, at least about 140, at least about 150, at least about 160, at least about 170, at least about 180, at least about 190, or at least about 200 amino acids in length.

In certain aspects, the scaffold protein comprises (i) GGKLSKKKKGYNVN (SEQ ID NO: 246), (ii) GAKLSKKKKGYNVN (SEQ ID NO: 247), (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254), (x) GGKLSKSGGSGGSV (SEQ ID NO: 255), or (xi) GAKKSKKRFSFKKS (SEQ ID NO: 256).

In some aspects, the scaffold protein does not comprise Met at the N terminus. In certain aspects, the scaffold protein comprises a myristoylated amino acid residue at the N terminus of the scaffold protein. In some aspects, the amino acid residue at the N terminus of the scaffold protein is Gly. In certain aspects, the amino acid residue at the N terminus of the scaffold protein is synthetic. In some aspects, the amino acid residue at the N terminus of the scaffold protein is a glycine analog.

In some aspects, an EV disclosed herein (e.g., exosome) further comprises a therapeutic molecule, an immune modulator, an adjuvant, or any combination thereof. In certain aspects, the therapeutic molecule comprises an antigen. In certain aspects, the therapeutic molecule comprises an immunosuppressive agent. In some aspects, the immunosuppressive agent comprises an antisense oligonucleotide.

In some aspects, an adjuvant is a Stimulator of Interferon Genes (STING) agonist, a toll-like receptor (TLR) agonist, an inflammatory mediator, or any combination thereof. In certain aspects, the adjuvant is a STING agonist. In some aspects, the STING agonist comprises a cyclic dinucleotide STING agonist or a non-cyclic dinucleotide STING agonist.

In some aspects, an adjuvant is a TLR agonist. In certain aspects, the TLR agonist comprises a TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), or any combination thereof.

In some aspects, the immune modulator comprises a cytokine. In certain aspects, the cytokine comprises an interferon. In some aspects, the EV is an exosome.

In some aspects, the therapeutic molecule, an immune modulator, an adjuvant, or any combination thereof, is associated with Scaffold X or Scaffold Y or a combination thereof. In some aspects, the therapeutic molecule is associated with a Scaffold X protein. In certain aspects, the therapeutic molecule is associated with a Scaffold Y protein. In some aspects, the immune modulator is associated with a Scaffold X protein. In some aspects, immune modulator is associated with a Scaffold Y protein. In some aspects, wherein the adjuvant is associated with a Scaffold X protein. In certain aspects, wherein the adjuvant is associated with a Scaffold Y protein.

Also disclosed herein is a pharmaceutical composition comprising the EV of the present disclosure and a pharmaceutically acceptable carrier. The present disclosure also provides a cell that produces an EV disclosed herein. Also disclosed herein is a cell comprising one or more vectors, wherein the vectors comprise a nucleic acid sequence encoding a targeting moiety disclosed herein. Provided herein is a kit comprising the EV of the present disclosure. The present disclosure further provides a method of making EVs (e.g., exosomes) comprising culturing a cell disclosed herein under a suitable condition and obtaining the EVs.

Present disclosure further provides a method of preventing or treating a disease in a subject in need thereof, comprising administering to the subject an EV disclosed herein or a pharmaceutical composition disclosed herein. In some aspects, the disease is selected from a cancer, a hemophilia, diabetes, a growth factor deficiency, an eye disease, a graft-versus-host disease (GvHD), an autoimmune disease, a gastrointestinal disease, a cardiovascular disease, a respiratory disease, an allergic disease, a degenerative disease, an infectious disease, fibrotic diseases, or any combination thereof.

Also provided herein is a method of delivering an EV to a subject, comprising administering to the subject an EV of the present disclosure.

In some aspects, an EV disclosed herein is administered parenterally, orally, intravenously, intramuscularly, intra-tumorally, intranasally, subcutaneously, or intraperitoneally. In some aspects, method of preventing or treating a disease or a method of delivering an EV to a subject, comprises administering an additional therapeutic agent.

### BRIEF DESCRIPTION OF FIGURES

FIG. 1 provides a schematic of four different exemplary anti-Clec9a targeting moieties disclosed herein. The targeting moieties include the following (from left to right): (i) Clec9a-specific single-chain Fv antibody fragment linked to a full-length Scaffold X protein (“scFv_FLPrX”); (ii) Clec9a-specific single-chain FV antibody fragment linked to a truncated Scaffold X protein (“scFv_short PrX”); (iii) Clec9a-specific single chain F(ab) antibody fragment linked to a full-length Scaffold X protein (“scFab_FLPrX”); and (iv) Clec9a-specific single-chain F(ab) antibody fragment linked to a truncated Scaffold X protein (“scFab_short PrX”). In each of the constructs, the truncated or full-length Scaffold X protein is conjugated to a tag (e.g., FLAG, GFP, or mCherry), which can be used to detect the expression of the targeting moieties.

FIG. 2A shows a flow cytometry histogram comparing the expression pattern of four different anti-Clec9a (“aClec9a”) constructs after transfection in HEK293 cells. The constructs shown include one of the four anti-Clec9a targeting moieties described in FIG. 1. The expression level of different anti-CLEC9a constructs was measured by GFP expression. Non-transfected HEK293 cells were used as the negative control.

FIG. 2B shows a comparison of the intensity of GFP expression in the exosomes produced from the transfected HEK293 cells described in FIG. 2A. Specifically, the exosomes expressed one of the following targeting moieties: (i) (i) Clec9a-specific single-chain Fv antibody fragment linked to a full-length Scaffold X protein (“scFv_FLPrX”); (ii) Clec9a-specific single-chain FV antibody fragment linked to a truncated Scaffold X protein (“scFv_SLPrX”); (iii) Clec9a-specific single chain F(ab) antibody fragment linked to a full-length Scaffold X protein (“scFab_FLPrX”); and (iv) Clec9a-specific single-chain F(ab) antibody fragment linked to a truncated Scaffold X protein (“scFab_SLPrX”). The fluorescence intensity corresponds to the relative amount of the target moiety expressed in the exosomes. As a control, exosomes that expressed Scaffold X protein alone (i.e., no anti-Clec9a targeting moiety) were used.

FIGS. 3A, 3B, and 3C show the binding affinity of the different anti-CLEC9a exosome constructs to both human and mouse Clec9A proteins, as measured by OCTET analysis. FIGS. 3A and 3B provide the binding results to human and mouse Clec9A, respectively. FIG. 3C shows the binding results of the positive (i.e., 10b4 antibody (bottom line) and negative controls (i.e., exosome expressing Scaffold X protein alone (top line) used.

FIG. 4 provides the flow cytometry gating strategy used to distinguish different human DC subsets. Specifically, the conventional DC population was identified by gating on CD11c⁺ and HLA-DR^{high} population. This population was then further divided into (i) CD1c⁻ and CD141⁺ (for cDC1) and (ii) CDlc⁺ and CD141⁻ (for cDC2). To identify pDC subset, cells were gated on CD11c⁺, HLA-DR^{intermediate}, and CD123⁺ population.

FIGS. 5A, 5B, 5C, and 5D show the uptake of anti-Clec9A-expressing exosomes in different dendritic cell populations from four different human donors, respectively. In each of FIGS. 5A-5D, the upper graph corresponds to the negative control (i.e., exosomes expressing Scaffold X protein alone), and the lower graph corresponds to the exosomes expressing anti-Clec9A. For each of the donors (i.e., donors 1, 2, 3, and 4), the uptake of the exosomes is shown for three different dendritic cell (DC) populations: plasmacytoid DC (“pDCs”), conventional DC 1 (“cDC1”), and conventional DC 2 (“cDC2”). The uptake of the exosomes is shown as the mean fluorescence index (MFI) of GFP expression. The results are shown as mean±S.E.M.

FIG. 6 provides the flow cytometry gating strategy used to distinguish different mouse DC subsets. Specifically, pDCs were identified by gating on CD137⁺, XCR1⁻, and Sirpa population. cDC1 cells were identified by gating on XCR1⁺, Clec9a⁺, and Sirpa⁻ population. cDC2 cells were identified by gating on CD8⁺, CD11b⁺, Sirpa⁺, XCR1⁻, and CD1c,b⁺.

FIGS. 7A and 7B show a comparison of exosome uptake by three different mouse dendritic cell populations: plasmacytoid DC (“pDCs+”), conventional DC 1 (“cDC1+”), and conventional DC 2 (“cDC2+”). FIG. 7A shows the uptake of anti-Clec9A-expressing exosomes.

FIG. 7B shows the uptake of the control exosomes (i.e., expressing Scaffold X protein alone). The uptake of the exosomes is shown as the mean fluorescence index (MFI) of GFP expression. The results are shown as mean±S.E.M.

FIGS. 8A and 8B show a comparison of the downregulation of anti-Clec9A in cDC1s (FIG. 8A) and pDCs (FIG. 8B) after treatment with anti-Clec9A-expressing exosomes. FIG. 8C shows the effect of anti-Clec9A-expressing exosomes on the expression pattern of XCR1 protein in cDC1s. The DCs shown were either unstimulated (triangle) or treated with one of the following exosomes: (i) control exosomes (i.e., expressing PrX alone) (“PrX”) or (ii) anti-Clec9A-expressing exosomes (“aClec9a”). The uptake of the exosomes is shown as the mean fluorescence index (MFI) of GFP expression. The results are shown as mean±S.E.M.

FIGS. 9A and 9B show Clec9a protein expression in HEK cells transfected with mouse Clec9a and human Clec9a, respectively. The Clec9a protein expression was measured using three different antibodies: (i) anti-mouse Clec9a (clone 10b4) (“(2)”), (ii) anti-mouse Clec9a (clone 7h11) (“(3)”), and (iii) anti-human Clec9a (clone E8F) (“(4)”). Unstained cells were used as a negative control (“(1)”).

FIGS. 10A and 10B show the exosome uptake by HEK cells transfected with human Clec9a at 4 hours and 24 hours post exosome treatment. The different groups shown include: (i) non-transfected HEK cells treated with control exosomes (i.e., expressing only Scaffold X protein) (“Native Cells—PrX EVs”; circle); (ii) non-transfected HEK cells treated with anti-Clec9a-expressing exosomes (“Native Cells—aClec9a EVs”; square); (iii) transfected HEK cells treated with control exosomes (“Clec9a Cells—Prx”; triangle); and (iv) transfected HEK cells treated with anti-Clec9a-expressing exosomes (“Clec9a Cells—aClec9a EVs”; inverted triangle). The uptake of the exosomes is shown as the mean fluorescence index (MFI) of mCherry expression. The results are shown as mean±S.E.M.

FIG. 11 shows the exosome uptake by HEK cells transfected with mouse Clec9a at 6 hours post exosome treatment. The different groups shown include: (i) non-transfected HEK cells treated with control exosomes (i.e., expressing only Scaffold X protein) (“Native Cells—PrX EVs”; circle); (ii) non-transfected HEK cells treated with anti-Clec9a-expressing exosomes (“Native Cells—aClec9a EVs”; square); (iii) transfected HEK cells treated with control exosomes (“Clec9a Cells—Prx”; triangle); and (iv) transfected HEK cells treated with anti-Clec9a-expressing exosomes (“Clec9a Cells—aClec9a EVs”; inverted triangle). The uptake of the exosomes is shown as the mean fluorescence index (MFI) of GFP expression. The results are shown as mean±S.E.M.

FIGS. 12A, 12B, 12C, 12D, 12E, 12F, 12G, and 12H show the immune cell distribution of exosomes expressing anti-Clec9a after intravenous administration into B16F10 tumor bearing mice. FIG. 12A provides the administration schedule. FIG. 12B shows the different treatment groups, including the Group #(“Grp.”), administered composition (“Drug”), dose of administration (“Dose”), route of administration (“Route”), and the number of animals in each group (“N”). FIGS. 12C and 12D show the uptake of the anti-Clec9a exosomes by different cell populations within the blood. The cell populations analyzed in the blood include the following: (i) conventional DC 1 (cDC1), (ii) conventional DC 2 (cDC2), (iii) plasmacytoid DC (pDC), (iv) CD11b+ Ly6C+ (monocytes); (v) CD11b+ Ly6G+ (neutrophils); and (vi) T, NK, and B cells. FIGS. 12E and 12F show the uptake of the anti-Clec9a exosomes by different cell populations within the spleen. The cell populations analyzed in the spleen include the following: (i) conventional DC 1 (cDC1), (ii) conventional DC 2 (cDC2), (iii) plasmacytoid DC (pDC), (iv) CD11b+ Ly6C+ (monocytes); (v) CD11b+ Ly6G+ (neutrophils); (vi) macrophages; (vii) red pulp macrophages; and (vi) T, NK, and B cells. FIGS. 12G and 12H show the uptake of the anti-Clec9a exosomes by different cell populations within the tumor. The cell populations analyzed include the following: (i) conventional DC1 (CD24+CD11b−); (ii) conventional DC2 (CD24−CD11b+), (iii) plasmacytoid DC (pDC); and (iv) macrophages. FIGS. 12C, 12E, and 12G show exosome uptake as a percentage of Cy5 positive cells within each population. FIGS. 12D, 12F, and 12H show the amount of exosomes that were taken up by the different cells by measuring the mean fluorescence intensity (MFI) of Cy5 expression.

FIGS. 13A, 13B, 13C, 13D, 13E, 13F, 13G, 13H, 13I, and 13J show the immune cell distribution of anti-Clec9a exosomes in CT26 tumor bearing mice after intravenous or intraturmoral administration. FIG. 13A provides the administration schedule. FIG. 13B shows the different treatment groups, including the Group #(“Grp.”), administered composition (“Drug”), dose of administration (“Dose”), route of administration (“Route”), the number of animals in each group (“N”), and the volume of administration (“Volume”). FIGS. 13C and 13D show exosome uptake by different cells within the blood after intravenous administration. The cell populations analyzed in the blood include the following: (i) conventional DC 1 (cDC1), (ii) conventional DC 2 (cDC2), (iii) plasmacytoid DC (pDC), (iv) CD11b+ Ly6C+ (monocytes); (v) CD11b+ Ly6G+ (neutrophils); and (vi) T, NK, and B cells. FIGS. 13E and 13F show exosome uptake by different cells within the spleen after intravenous administration. The cell populations analyzed in the spleen include the following: (i) conventional DC 1 (cDC1), (ii) conventional DC 2 (cDC2), (iii) CD11b+ Ly6C+ (monocytes); (iv) CD11b+ Ly6G+ (neutrophils); (v) F4/80+ (macrophages); (vi) red pulp macrophages; and (vii) T, NK, and B cells. FIGS. 13G and 13H show exosome uptake by different cells within the tumor after intravenous administration. The cell populations analyzed include the following: (i) conventional DC1 (CD24+CD11b−); (ii) conventional DC2 (CD24+CD11b+), (iii) plasmacytoid DC (pDC); and (iv) macrophages. FIGS. 13I and 13J show exosome uptake by different cells within the tumor after intratumoral administration. The cell populations analyzed include the following: (i) conventional DC1 (CD24+CD11b−); (ii) conventional DC2 (CD24+CD11b+), (iii) plasmacytoid DC (pDC); and (iv) macrophages. FIGS. 13C, 13E, 13G, and 13I show exosome uptake as a percentage of Cy5 positive cells within each population. FIGS. 13D, 13F, 13H, and 13J show the amount of exosomes that were taken up by the different cells by measuring the mean fluorescence intensity (MFI) of Cy5 expression.

FIGS. 14A, 14B, and 14C show the uptake of anti-Clec9a exosomes loaded with STING agonist in different HEK-Blue STING reporter cell lines. FIG. 14A shows uptake in native (i.e., not modified to express Clec9a) HEK-Blue cell line. FIG. 14B shows uptake in HEK-Blue cell line modified to express mouse Clec9a. FIG. 14C shows uptake in HEK-Blue cell line modified to express human Clec9a. In each of FIGS. 14A-14C, the following were used as controls: (i) soluble STING agonist (“Free STING”); (ii) EVs expressing Scaffold X alone (“Prx”); and (iii) isotype control (i.e., an exosome expressing an irrelevant antibody).

FIGS. 15A and 15B show a comparison of the uptake of anti-Clec9a exosomes loaded with STING agonist in two different STING RAW-reporting cell lines. In FIG. 15A, uptake is shown in a native STING Raw-reporting cell line, which does not express Clec9a. In FIG. 15B, uptake is shown in a STING Raw-reporting cell line modified to express Clec9a. In both FIGS. 15A and 15B, uptake of the exosomes are measured based on STING luciferase reporter activity. The following grounds are shown in each of the figures: (i) soluble STING agonist (“Free STING”) (circle), (ii) STING-loaded exosomes expressing Scaffold X alone (“Prx-STING”) (triangle); (iii) STING-loaded exosomes expressing anti-Clec9a linked to Scaffold X (“aClec9a-STING:) (closed inverted triangle); (iv) STING-loaded exosomes expressing an isotype control antibody (“Isotype-STING”) (open inverted triangle); and (v) exosomes loaded only with STING agonist (“Native-STING”) (square).

FIGS. 16A and 16B show the level of IFN-β produced after stimulation of total mouse splenocytes (FIG. 16A) or isolated mouse splenic dendritic cells (FIG. 16B) with anti-Clec9a exosomes loaded with STING agonist (“aClec9a-STING”) (inverted triangle). In both FIGS. 16A and 16B, the following were used as controls: (i) no stimulation (“-”) (circle); (ii) soluble STING agonist (“free STING”) (square); (iii) STING-loaded exosomes expressing Scaffold X alone (“PrX-STING”) (triangle); and (iv) STING-loaded exosomes expressing an isotype control antibody (“Isotype-STING”) (diamond).

FIGS. 17A, 17B, 17C, 17D, 17E, and 17F show the levels of CXCL10, CCL5, and IL-6 produced after stimulation of total mouse splenocytes or isolated mouse splenic dendritic cells with anti-Clec9a exosomes loaded with STING agonist (“aClec9a-STING”) (inverted triangle). FIGS. 17A, 17B, and 17C show the levels of CXCL10, CCL5, and IL-6 measured using total mouse splenocytes, respectively. FIGS. 17D, 17E, and 17F show the levels of CXCL10, CCL5, and IL-6 measured using isolated mouse splenic dendritic cells, respectively. The controls are the same as those used in FIGS. 16A and 16B.

FIGS. 18A, 18B, 18C, 18D, 18E, and 18F show the levels of IL-12, IFN-α, and TNF-α produced after stimulation of total mouse splenocytes or isolated mouse splenic dendritic cells with anti-Clec9a exosomes loaded with STING agonist (“aClec9a-STING”) (inverted triangle). FIGS. 18A, 18B, and 18C show the levels of IL-12, IFN-α, and TNF-α measured using total splenocytes, respectively. FIGS. 18D, 18E, and 18F show the levels of IL-12, IFN-α, and TNF-α measured using isolated dendritic cells, respectively. The controls are the same as those used in FIGS. 16A and 16B.

FIGS. 19A, 19B, 19C, 19D, 19E, 19F, 19G, and 19G show the levels of GMC-SF, IFN-γ, IL-10, and CCL-2 produced after stimulation of total mouse splenocytes or isolated mouse splenic dendritic cells with anti-Clec9a exosomes loaded with STING agonist (“aClec9a-STING”) (inverted triangle). FIGS. 19A, 19B, 19C, and 19D show the levels of GMC-SF, IFN-γ, IL-10, and CCL-2 measured using total splenocytes, respectively. FIGS. 19E, 19F, 19G, and 19G show the levels of GMC-SF, IFN-γ, IL-10, and CCL-2 measured using isolated dendritic cells, respectively. The controls are the same as those used in FIGS. 16A and 16B.

FIGS. 20A, 20B, 20C, and 20D show the levels of IL-1β and CXCL1 produced after stimulation of total mouse splenocytes or isolated mouse splenic dendritic cells with anti-Clec9a exosomes loaded with STING agonist (“aClec9a-STING”) (inverted triangle). FIGS. 20A and 20B show the levels of IL-1β and CXCL1 measured using total splenocytes, respectively. FIGS. 20C and 20D show the levels of IL-1β and CXCL1 measured using isolated dendritic cells, respectively. The controls are the same as those used in FIGS. 16A and 16B.

FIG. 21 provides a schematic of the experimental design for assessing the efficacy of anti-Clec9a exosomes in a tumor model.

### DETAILED DESCRIPTION OF DISCLOSURE

The present disclosure is directed to an EV (e.g., exosome) comprising a targeting moiety that is not naturally expressed in the EV and can specifically target the EV to an immune cell, such as a dendritic cell. In some aspects, the targeting moiety specifically binds to a marker expressed on the immune cell. Non-limiting examples of the various aspects are shown in the present disclosure.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In some aspects, an EV comprises one or more payloads or other exogenous biologically active molecules. In some aspects, an EV comprises a targeting moiety that is exogenous to the EV (i.e., not naturally expressed in the EV) and that allows the EV to target a specific population of immune cells (e.g., dendritic cells). In certain aspects, an extracellular vehicle can further comprise one or more scaffold moieties. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products. The EVs disclosed herein have been modified and therefore, do not comprise naturally occurring EVs.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. In some aspects, an exosome comprises one or more exogenous biologically active molecules (e.g., as described herein). In some aspects, an exosome disclosed herein comprises a targeting moiety that is exogenous to the exosome (i.e., not naturally expressed in the exosome) and that allows the exosome to target a specific population of immune cells (e.g., dendritic cells). In certain aspects, an exosome further comprises one or more scaffold moieties. As described infra, exosomes can be derived from a producer cell, and isolated from the producer cell based on its size, density, biochemical parameters, or a combination thereof. In some aspects, exosomes of the present disclosure are produced by cells that express one or more transgene products. The exosomes of the present disclosure are modified and therefore, do not comprise naturally occurring exosomes.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In some aspects, a nanovesicle comprises one or more exogenous biologically active molecules (e.g., disclosed herein). In some aspects, a nanovesicle can further comprise a targeting moiety that is exogenous to the nanovesicle (i.e., not naturally expressed in the nanovesicle) and that allows the nanovesicle to target a specific population of immune cells (e.g., dendritic cells). In certain aspects, a nanovesicle further comprises one or more scaffold moieties. Nanovesicles, once derived from a producer cell, can be isolated from the producer cell based on its size, density, biochemical parameters, or a combination thereof. As used herein, nanovesicles have been modified and therefore, do not comprise naturally occurring nanovesicles.

As used herein the term “surface-engineered EVs, e.g., exosomes” (e.g., Scaffold X-engineered EVs, e.g., exosomes) refers to an EV (e.g., exosome) with the membrane or the surface modified in its composition, so that the membrane or the surface of the engineered EV (e.g., exosome), is different from either that of the EV prior to the modification or of the naturally occurring EV. The engineering can be on the surface of the EV (e.g., exosome) or in the membrane of the EV (e.g., exosome) so that the surface of the EV, e.g., exosome, is changed. For example, the membrane is modified in its composition of a protein, a lipid, a small molecule, a carbohydrate, etc. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV, e.g., exosome, comprises one or more exogenous biologically active molecules. In certain aspects, the exogenous biologically active molecules can comprise an exogenous protein (i.e., a protein that the EV, e.g., exosome, does not naturally express) or a fragment or variant thereof that can be exposed to the surface of the EV, e.g., exosome, or can be an anchoring point (attachment) for a moiety exposed on the surface of the EV, e.g., exosome. In other aspects, a surface-engineered EV, e.g., exosome, comprises a higher expression (e.g., higher number) of a natural exosome protein (e.g., Scaffold X) or a fragment or variant thereof that can be exposed to the surface of the EV, e.g., exosome, or can be an anchoring point (attachment) for a moiety exposed on the surface of the EV, e.g., exosome.

As used herein the term “lumen-engineered exosome” (e.g., Scaffold Y-engineered exosome) refers to an EV, e.g., exosome, with the membrane or the lumen of the EV, e.g., exosome, modified in its composition so that the lumen of the engineered EV, e.g., exosome, is different from that of the EV, e.g., exosome, prior to the modification or of the naturally occurring EV, e.g., exosome. The engineering can be directly in the lumen or in the membrane of the EV, e.g., exosome so that the lumen of the EV, e.g., exosome is changed. For example, the membrane is modified in its composition of a protein, a lipid, a small molecule, a carbohydrate, etc. so that the lumen of the EV, e.g., exosome is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered exosome comprises one or more exogenous biologically active molecules. In certain aspects, the exogenous biologically active molecules can comprise an exogenous protein (i.e., a protein that the EV, e.g., exosome does not naturally express) or a fragment or variant thereof that can be exposed in the lumen of the EV, e.g., exosome or can be an anchoring point (attachment) for a moiety exposed on the inner layer of the EV, e.g., exosome. In other aspects, a lumen-engineered EV, e.g., exosome, comprises a higher expression of a natural exosome protein (e.g., Scaffold X or Scaffold Y) or a fragment or variant thereof that can be exposed to the lumen of the exosome or can be an anchoring point (attachment) for a moiety exposed in the lumen of the exosome.

The term “modified,” when used in the context of EVs, e.g., exosomes described herein, refers to an alteration or engineering of an EV, e.g., exosome and/or its producer cell, such that the modified EV, e.g., exosome is different from a naturally-occurring EV, e.g., exosome. In some aspects, a modified EV, e.g., exosome described herein comprises a membrane that differs in composition of a protein, a lipid, a small molecular, a carbohydrate, etc. compared to the membrane of a naturally-occurring EV, e.g., exosome (e.g., membrane comprises higher density or number of natural exosome proteins and/or membrane comprises multiple (e.g., at least two) biologically active molecules that are not naturally found in exosomes (e.g., therapeutic molecules (e.g., antigen), targeting moiety, adjuvant, and/or immune modulator). As used herein, biologically active molecules that are not naturally found in exosomes are also described as “exogenous biologically active molecules.”. In certain aspects, such modifications to the membrane changes the exterior surface of the EV, e.g., exosome (e.g., surface-engineered EVs, e.g., exosomes described herein). In certain aspects, such modifications to the membrane changes the lumen of the EV, e.g., exosome (e.g., lumen-engineered EVs, e.g., exosomes described herein).

As used herein, the terms “binding moiety,” “bio-distribution modifying agent,” and “targeting moiety” are interchangeable and refer to an agent that can modify the distribution of extracellular vesicles (e.g., exosomes, nanovesicles) in vivo or in vitro (e.g., in a mixed culture of cells of different varieties). The targeting moiety can be a biological molecule, such as a protein, a peptide, a lipid, or a synthetic molecule. For example, the targeting moiety can be an antibody (e.g., anti-CD22 nanobody), a synthetic polymer (e.g., PEG), a natural ligand (e.g., CD40L, albumin), a recombinant protein (e.g., XTEN), but not limited thereto. Without being bound to any particular theory, a targeting moiety disclosed herein can modify the distribution of an EV (e.g., exosome) by binding to a marker (also referred to herein as a “target molecule”) expressed on a specific cell type (e.g., a cancer cell or a cell specific to a certain tissue). In some aspects, a targeting moiety disclosed herein binds to a marker for a specific population of immune cells (e.g., dendritic cells). In certain aspects, the marker is expressed only on the population of immune cells of interest. In some aspects, a marker comprises a C-type lectin domain family 9 member A (CLEC9A) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), or any combination thereof. In certain aspects, the targeting moiety is displayed on the surface of EVs (e.g., exosomes). In some aspects, the targeting moiety can be displayed on the EV surface by being fused to a scaffold protein (e.g., Scaffold X) (e.g., as a genetically encoded fusion molecule). In other aspects, the targeting moiety can be displayed on the EV surface by chemical reaction attaching the targeting moiety to an EV surface molecule. A non-limiting example is PEGylation. In some aspects, a targeting moiety disclosed herein can be combined with a functional moiety, such as a small molecule (e.g., STING, ASO), a drug, and/or a therapeutic protein (e.g., anti-mesothelin antibody/pro-apoptotic proteins).

As used herein, the term “C-type lectin domain family 9 member A” (Clec9a) protein refers to a group V C-type lectin-like receptor (CTLR) that functions as an activation receptor and is expressed on myeloid lineage cells (e.g., DCs). Huysamen et al., J Biol Chem 283(24):16693-701 (2008); U.S. Pat. No. 9,988,431 B2, each of which is herein incorporated by reference in its entirety. Synonyms of Clec9a are known and include CD370, DNGR-1, 5B5, HEEE9341, and C-type lectin domain containing 9A. In some aspects, Clec9a protein is expressed on human cDC1 cells. In some aspects, Clec9a protein is expressed on mouse cDC1 and pDC cells. Unless indicated otherwise, Clec9a, as used herein, can refer to Clec9a from one or more species (e.g., humans, non-human primates, dogs, cats, guinea pigs, rabbits, rats, mice, horses, cattle, and bears).

As used herein, the term “C-type lectin like domain” (CTLD) refers to protein domain that are homologous to the carbohydrate recognition domain (CRD) of the C-type lectins, or which have structure resembling the structure of the prototypic C-type lectin CRD.” Zelensky et al., FEBS J 272(24):6179-217 (2005). Initially, the CTLD domain was identified as a domain common to the so-called C-type lectins (calcium-dependent carbohydrate binding proteins) and named “Carbohydrate Recognition Domain” (“CRD”). More recently, it has become evident that this domain is shared among many eukaryotic proteins, of which several do not bind sugar moieties, and hence, the canonical domain has been named as CTLD. CTLDs have been reported to bind a wide diversity of compounds, including carbohydrates, lipids and proteins. The CTLD consists of approximately 120 amino acid residues and, characteristically, contains two or three intra-chain disulfide bridges. Although the similarity at the amino acid sequence level between CTLDs from different proteins is relatively low, the 3D-structures of a number of CTLDs have been found to be highly conserved, with the structural variability essentially confined to a so-called loop-region, often defined by up to five loops. See U.S. Publication No. 2013/0273150, which is herein incorporated by reference in its entirety.

As used herein, the term “C-type lectins” refers to a superfamily of proteins containing at least one C-type lectin domain (CTLD), which have been classified into groups depending on the arrangement of their CTLDs. Huysamen et al., J Biol Chem 283(24):16693-701 (2008). Currently, C-type lectins have been categorized into 17 different C-type lectin groups (Groups I-XVII). Zelensky et al., FEBS J 272(24):6179-217 (2005).

As used herein, the term “scaffold moiety” refers to a molecule that can be used to anchor a payload or any other exogenous biologically active molecule of interest (e.g., targeting moiety, adjuvant, and/or immune modulator) to the EV, e.g., exosome, either on the luminal surface or on the exterior surface of the EV, e.g., exosome. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV, e.g., exosome. In some aspects, a scaffold moiety comprises a lipid, carbohydrate, or protein that does not naturally exist in the EV, e.g., exosome. In certain aspects, a scaffold moiety is Scaffold X. In some aspects, a scaffold moiety is Scaffold Y. In further aspects, a scaffold moiety comprises both Scaffold X and Scaffold Y. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); CD9, CD63, CD81, PDGFR, GPI anchor proteins, lactadherin, LAMP2, and LAMP2B.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV, e.g., exosome). In some aspects, a Scaffold X can anchor an exogenous protein (e.g., those disclosed herein, e.g., targeting moiety, therapeutic molecule, adjuvant, and/or immune modulator) to the external surface or the luminal surface of the exosome.

As used herein, the term “Scaffold Y” refers to exosome proteins that were newly identified within the lumen of exosomes. See, e.g., International Appl. No. PCT/US2018/061679, which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety to the luminal surface of the exosome). In some aspects, a Scaffold Y can anchor an exogenous protein (e.g., those disclosed herein, e.g., targeting moiety, therapeutic molecule, adjuvant, and/or immune modulator) to the luminal surface of the EV, e.g., exosome.

As used herein, the term “fragment” of a protein (e.g., therapeutic protein, Scaffold X, or Scaffold Y) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface or on the exterior surface of the EV, e.g., exosome. Similarly, in certain aspects, a functional fragment of a Scaffold Y protein retains the ability to anchor a moiety on the luminal surface of the EV, e.g., exosome. Whether a fragment is a functional fragment can be assessed by any art known methods to determine the protein content of EVs, e.g., exosomes including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein. In some aspects, a functional fragment of a Scaffold Y protein retains at least about 50%, at least about 60%, at least about 70%, at least about 80%, at least about 90% or at least about 100% of the ability, e.g., an ability to anchor another molecule, of the naturally occurring Scaffold Y protein.

As used herein, the term “variant” of a molecule (e.g., functional molecule, therapeutic molecule, Scaffold X and/or Scaffold Y) refers to a molecule that shares certain structural and functional identities with another molecule upon comparison by a method known in the art. For example, a variant of a protein can include a substitution, insertion, deletion, frameshift or rearrangement in another protein.

In some aspects, a variant of a Scaffold X comprises a variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins. In some aspects, variants or variants of fragments of PTGFRN share at least about 70%, at least about 80%, at least about 85%, at least about 90%, at least about 95%, at least about 96%, at least about 97%, at least about 98%, or at least about 99% sequence identity with PTGFRN according to SEQ ID NO: 1 or with a functional fragment thereof. In some aspects variants or variants of fragments of BSG share at least about 70%, at least about 80%, at least about 85%, at least about 90%, at least about 95%, at least about 96%, at least about 97%, at least about 98%, or at least about 99% sequence identity with BSG according to SEQ ID NO: 9 or with a functional fragment thereof. In some aspects variants or variants of fragments of IGSF2 share at least about 70%, at least about 80%, at least about 85%, at least about 90%, at least about 95%, at least about 96%, at least about 97%, at least about 98%, or at least about 99% sequence identity with IGSF2 according to SEQ ID NO: 34 or with a functional fragment thereof. In some aspects variants or variants of fragments of IGSF3 share at least about 70%, at least about 80%, at least about 85%, at least about 90%, at least about 95%, at least about 96%, at least about 97%, at least about 98%, or at least about 99% sequence identity with IGSF3 according to SEQ ID NO: 20 or with a functional fragment thereof. In some aspects variants or variants of fragments of IGSF8 share at least about 70%, at least about 80%, at least about 85%, at least about 90%, at least about 95%, at least about 96%, at least about 97%, at least about 98%, or at least about 99% sequence identity with IGSF8 according to SEQ ID NO: 14 or with a functional fragment thereof. In some aspects variants or variants of fragments of ITGB1 share at least about 70%, at least about 80%, at least about 85%, at least about 90%, at least about 95%, at least about 96%, at least about 97%, at least about 98%, or at least about 99% sequence identity with ITGB1 according to SEQ ID NO: 21 or with a functional fragment thereof. In some aspects variants or variants of fragments of ITGA4 share at least about 70%, at least about 80%, at least about 85%, at least about 90%, at least about 95%, at least about 96%, at least about 97%, at least about 98%, or at least about 99% sequence identity with ITGA4 according to SEQ ID NO: 22 or with a functional fragment thereof. In some aspects variants or variants of fragments of SLC3A2 share at least about 70%, at least about 80%, at least about 85%, at least about 90%, at least about 95%, at least about 96%, at least about 97%, at least about 98%, or at least about 99% sequence identity with SLC3A2 according to SEQ ID NO: 23 or with a functional fragment thereof. In some aspects variants or variants of fragments of ATP1A1 share at least about 70%, at least about 80%, at least about 85%, at least about 90%, at least about 95%, at least about 96%, at least about 97%, at least about 98%, or at least about 99% sequence identity with ATP1A1 according to SEQ ID NO: 24 or with a functional fragment thereof. In some aspects variants or variants of fragments of ATP1A2 share at least about 70%, at least about 80%, at least about 85%, at least about 90%, at least about 95%, at least about 96%, at least about 97%, at least about 98%, or at least about 99% sequence identity with ATP1A2 according to SEQ ID NO: 25 or with a functional fragment thereof. In some aspects variants or variants of fragments of ATP1A3 share at least about 70%, at least about 80%, at least about 85%, at least about 90%, at least about 95%, at least about 96%, at least about 97%, at least about 98%, or at least about 99% sequence identity with ATP1A3 according to SEQ ID NO: 26 or with a functional fragment thereof. In some aspects variants or variants of fragments of ATP1A4 share at least about 70%, at least about 80%, at least about 85%, at least about 90%, at least about 95%, at least about 96%, at least about 97%, at least about 98%, or at least about 99% sequence identity with ATP1A4 according to SEQ ID NO: 27 or with a functional fragment thereof. In some aspects variants or variants of fragments of ATP1B3 share at least about 70%, at least about 80%, at least about 85%, at least about 90%, at least about 95%, at least about 96%, at least about 97%, at least about 98%, or at least about 99% sequence identity with ATP1B3 according to SEQ ID NO: 28 or with a functional fragment thereof. In some aspects variants or variants of fragments of ATP2B1 share at least about 70%, at least about 80%, at least about 85%, at least about 90%, at least about 95%, at least about 96%, at least about 97%, at least about 98%, or at least about 99% sequence identity with ATP2B1 according to SEQ ID NO: 29 or with a functional fragment thereof. In some aspects variants or variants of fragments of ATP2B2 share at least about 70%, at least about 80%, at least about 85%, at least about 90%, at least about 95%, at least about 96%, at least about 97%, at least about 98%, or at least about 99% sequence identity with ATP2B2 according to SEQ ID NO: 30 or with a functional fragment thereof. In some aspects variants or variants of fragments of ATP2B3 share at least about 70%, at least about 80%, at least about 85%, at least about 90%, at least about 95%, at least about 96%, at least about 97%, at least about 98%, or at least about 99% sequence identity with ATP2B3 according to SEQ ID NO: 31 or with a functional fragment thereof. In some aspects variants or variants of fragments of ATP2B4 share at least about 70%, at least about 80%, at least about 85%, at least about 90%, at least about 95%, at least about 96%, at least about 97%, at least about 98%, or at least about 99% sequence identity with ATP2B4 according to SEQ ID NO: 32 or with a functional fragment thereof. In some aspects, the variant or variant of a fragment of Scaffold X protein disclosed herein retains the ability to be specifically targeted to EVs, e.g., exosomes. In some aspects, the Scaffold X includes one or more mutations, for example, conservative amino acid substitutions.

In some aspects, a variant of a Scaffold Y comprises a variant having at least 70% identity to MARCKS, MARCKSL1, BASP1 or a fragment of MARCKS, MARCKSL1, or BASP1.

In some aspects variants or variants of fragments of MARCKS share at least about 70%, at least about 80%, at least about 85%, at least about 90%, at least about 95%, at least about 96%, at least about 97%, at least about 98%, or at least about 99% sequence identity with MARCKS according to SEQ ID NO: 47 or with a functional fragment thereof. In some aspects variants or variants of fragments of MARCKSL1 share at least about 70%, at least about 80%, at least about 85%, at least about 90%, at least about 95%, at least about 96%, at least about 97%, at least about 98%, or at least about 99% sequence identity with MARCKSL1 according to SEQ ID NO: 48 or with a functional fragment thereof. In some aspects variants or variants of fragments of BASP1 share at least about 70%, at least about 80%, at least about 85%, at least about 90%, at least about 95%, at least about 96%, at least about 97%, at least about 98%, or at least about 99% sequence identity with BASP1 according to SEQ ID NO: 49 or with a functional fragment thereof. In some aspects, the variant or variant of a fragment of Scaffold Y protein retains the ability to be specifically targeted to the luminal surface of EVs, e.g., exosomes. In some aspects, the Scaffold Y includes one or more mutations, e.g., conservative amino acid substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X and/or Scaffold Y is modified at any convenient location.

As used herein the terms “linked to,” “conjugated to,” and “anchored to” are used interchangeably and refer to a covalent or non-covalent bond formed between a first moiety and a second moiety, e.g., Scaffold X and a targeting moiety disclosed herein.

The term “encapsulated”, or grammatically different forms of the term (e.g., encapsulation, or encapsulating), refers to a status or process of having a first moiety (e.g., exogenous biologically active molecule, e.g., therapeutic molecule, adjuvant, or immune modulator) inside a second moiety (e.g., an EV, e.g., exosome) without chemically or physically linking the two moieties. In some aspects, the term “encapsulated” can be used interchangeably with “in the lumen of”. Non-limiting examples of encapsulating a first moiety (e.g., exogenous biologically active molecule, e.g., therapeutic molecule, adjuvant, or immune modulator) into a second moiety (e.g., EVs, e.g., exosomes) are disclosed elsewhere herein.

As used herein, the term “producer cell” refers to a cell used for generating an EV, e.g., exosome. A producer cell can be a cell cultured in vitro, or a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells. In certain aspects, a producer cell is not an antigen-presenting cell. In some aspects, a producer cell is not a dendritic cell, a B cell, a mast cell, a macrophage, a neutrophil, Kupffer-Browicz cell, cell derived from any of these cells, or any combination thereof. In some aspects, the EVs, e.g., exosomes useful in the present disclosure do not carry an antigen on MHC class I or class II molecule exposed on the surface of the EV, e.g., exosome, but instead can carry an antigen in the lumen of the EV, e.g., exosome or on the surface of the EV, e.g., exosome by attachment to Scaffold X and/or Scaffold Y.

As used herein, an “MHC class I molecule” refers to a protein product of a wild-type or variant HLA class I gene encoding an MHC class I molecule. Accordingly, “HLA class I molecule” and “MHC class I molecule” are used interchangeably herein.

MHC class I molecules are one of two primary classes of major histocompatibility complex (MHC) molecules (the other being MHC class II) and are found on the cell surface of all nucleated cells in the bodies of jawed vertebrates. They also occur on platelets, but not on red blood cells. Their function is to display peptide fragments of proteins from within the cell to cytotoxic T cells; this will trigger an immediate response from the immune system against a particular non-self antigen displayed with the help of an MHC class I protein. Because MHC class I molecules present peptides derived from cytosolic proteins, the pathway of MHC class I presentation is often called cytosolic or endogenous pathway.

In humans, the HLAs corresponding to MHC class I are HLA-A, HLA-B, and HLA-C. The MHC Class I molecule comprises two protein chains: the alpha chain and the β2-microglobulin (β2m) chain. Human β2m is encoded by the B2M gene. Class I MHC molecules bind peptides generated mainly from degradation of cytosolic proteins by the proteasome. The MHC I:peptide complex is then inserted via endoplasmic reticulum into the external plasma membrane of the cell. The epitope peptide is bound on extracellular parts of the class I MHC molecule. Thus, the function of the class I MHC is to display intracellular proteins to cytotoxic T cells (CTLs). However, class I MHC can also present peptides generated from exogenous proteins, in a process known as cross-presentation.

A normal cell will display peptides from normal cellular protein turnover on its class I MHC, and CTLs will not be activated in response to them due to central and peripheral tolerance mechanisms. When a cell expresses foreign proteins, such as after viral infection, a fraction of the class I MHC will display these peptides on the cell surface. Consequently, CTLs specific for the MHC:peptide complex will recognize and kill presenting cells. Alternatively, class I MHC itself can serve as an inhibitory ligand for natural killer cells (NKs). Reduction in the normal levels of surface class I MHC, a mechanism employed by some viruses and certain tumors to evade CTL responses, activates NK cell killing.

As used herein, an “MHC class II molecule” refers to a protein product of a wild-type or variant HLA class II gene encoding an MHC class II molecule. Accordingly, “HLA class II molecule” and “MHC class II molecule” are used interchangeably herein.

MHC class II molecules are a class of major histocompatibility complex (MHC) molecules normally found only on professional antigen-presenting cells such as dendritic cells, mononuclear phagocytes, some endothelial cells, thymic epithelial cells, and B cells. These cells are important in initiating immune responses. The antigens presented by class II peptides are derived from extracellular proteins (not cytosolic as in MHC class I).

Like MHC class I molecules, class II molecules are also heterodimers, but in this case consist of two homogenous peptides, an α and β chain, both of which are encoded in the MHC. The subdesignation α1, α2, etc. refers to separate domains within the HLA gene; each domain is usually encoded by a different exon within the gene, and some genes have further domains that encode leader sequences, transmembrane sequences, etc. These molecules have both extracellular regions as well as a transmembrane sequence and a cytoplasmic tail. The α1 and β1 regions of the chains come together to make a membrane-distal peptide-binding domain, while the α2 and β2 regions, the remaining extracellular parts of the chains, form a membrane-proximal immunoglobulin-like domain. The antigen binding groove, where the antigen or peptide binds, is made up of two α-helixes walls and β-sheet. Because the antigen-binding groove of MHC class II molecules is open at both ends while the corresponding groove on class I molecules is closed at each end, the antigens presented by MHC class II molecules are longer, generally between 15 and 24 amino acid residues long. Loading of a MHC class II molecule occurs by phagocytosis; extracellular proteins are endocytosed, digested in lysosomes, and the resulting epitopic peptide fragments are loaded onto MHC class II molecules prior to their migration to the cell surface. In humans, the MHC class II protein complex is encoded by the human leukocyte antigen gene complex (HLA). HLAs corresponding to MHC class II are HLA-DP, HLA-DM, HLA-DOA, HLA-DOB, HLA-DQ, and HLA-DR. Mutations in the HLA gene complex can lead to bare lymphocyte syndrome (BLS), which is a type of MHC class II deficiency.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “immune modulator” refers to an agent that acts on a target (e.g., a target cell) that is contacted with the extracellular vesicle, and regulates the immune system. Non-limiting examples of immune modulator that can be introduced into an EV (e.g., exosome) and/or a producer cell include agents such as, modulators of checkpoint inhibitors, ligands of checkpoint inhibitors, cytokines, derivatives thereof, or any combination thereof. The immune modulator can also include an agonist, an antagonist, an antibody, an antigen-binding fragment, a polynucleotide, such as siRNA, miRNA, lncRNA, mRNA, DNA, or a small molecule.

As used herein, the term “payload” refers to an agent that acts on a target (e.g., a target cell) that is contacted with the EV. Non-limiting examples of payload that can be included on the EV, e.g., exosome, are a therapeutic molecule (e.g., antigen or immunosuppressive agent), an adjuvant, and/or an immune modulator. Payloads that can be introduced into an EV, e.g., exosome, and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises an exogenous biologically active molecule (e.g., those disclosed herein).

As used herein, the term “biologically active molecule” refers to an agent that has activity in a biological system (e.g., a cell or a human subject), including, but not limited to a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof can be natural, synthetic or humanized, a peptide hormone, a receptor, a signaling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which can be modified or unmodified; an amino acid or analogue thereof, which can be modified or unmodified; a non-peptide (e.g., steroid) hormone; a proteoglycan; a lipid; or a carbohydrate. In certain aspects, a biologically active molecule comprises a therapeutic molecule (e.g., an antigen), a targeting moiety (e.g., an antibody or an antigen-binding fragment thereof), an adjuvant, an immune modulator, or any combination thereof. In some aspects, the biologically active molecule comprises a macromolecule (e.g., a protein, an antibody, an enzyme, a peptide, DNA, RNA, or any combination thereof). In some aspects, the biologically active molecule comprises a small molecule (e.g., an antisense oligomer (ASO), an siRNA, STING, a pharmaceutical drug, or any combination thereof). In some aspects, the biologically active molecules are exogenous to the exosome, i.e., not naturally found in the exosome.

As used herein, the term “therapeutic molecule” refers to any molecule that can treat and/or prevent a disease or disorder in a subject (e.g., human subject).

In some aspects, a therapeutic molecule comprises an antigen. As used herein, the term “antigen” refers to any agent that when introduced into a subject elicits an immune response (cellular or humoral) to itself. In some aspects, an antigen is not expressed on major histocompatibility complex I and/or II molecules. In other aspects, while an antigen in the EV, e.g., exosome, is not expressed as MHC class I or II complex, the EV, e.g., exosome, can still contain MHC class I/II molecules on the surface of the EV, e.g., exosome. Accordingly, in certain aspects, EVs, e.g., exosomes, disclosed herein do not directly interact with T-cell receptors (TCRs) of T cells to induce an immune response against the antigen. Similarly, in certain aspects, EVs, e.g., exosomes, of the present disclosure do not transfer the antigen directly to the surface of the target cell (e.g., dendritic cell) through cross-dressing. Cross-dressing is a mechanism commonly used by EVs, e.g., exosomes, derived from dendritic cells (DEX) to induce T cell activation. See Pitt, J. M., et al., J Clin Invest 126(4): 1224-32 (2016). In other aspects, the EVs, e.g., exosomes, of the present disclosure are engulfed by antigen presenting cells and can be expressed on the surface of the antigen presenting cells as MHC class I and/or MHC class II complex.

In some aspects, a therapeutic molecule comprises an immunosuppressive agent. As used herein, the term “immunosuppressive agent” refers to any agent (e.g., therapeutic molecule) that slows or halts an immune response in a subject. Immunosuppressive agents can be given to a subject to prevent the subject's immune system from mounting an immune response after an organ transplant or for treating a disease that is caused by an overactive immune system. Examples of immunosuppressive agents include, but are not limited to, a calcineurin inhibitor, such as, but not limited to, cyclosporine, ISA(TX) 247, tacrolimus or calcineurin, a target of rapamycin, such as, but not limited to, sirolimus, everolimus, FK778 or TAFA-93, an interleukin-2 α-chain blocker, such as, but not limited to, basiliximab and daclizumab, an inhibitor of inosine monophosphate dehydrogenase, such as mycophenolate mofetil, an inhibitor of dihydrofolic acid reductase, such as, but not limited to, methotrexate, a corticosteroid, such as, but not limited to, prednisolone and methylprednisolone, or an immunosuppressive antimetabolite, such as, but not limited to, azathioprine. In certain aspects, an immunosuppressive agent comprises an antisense oligonucleotide. In some aspects, an EV disclosed herein (e.g., exosome) can comprise both an antigen and an immunosuppressive agent. Not to be bound by any one theory, an EV (e.g., exosome) comprising both an antigen and an immunosuppressive agent can be used to induce tolerance to the antigen.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects, the antibody or antigen-binding fragment thereof comprises a scFv, scFab, scFab-Fc, nanobody, or any combination thereof. In some aspects, the antibody or antigen-binding fragment thereof comprises an agonist antibody, a blocking antibody, a targeting antibody, a fragment thereof, or a combination thereof. In some aspects, the agonist antibody is a CD40L agonist. In some aspects, the blocking antibody binds a target protein selected from programmed death 1 (PD-1), programmed death ligand 1 (PD-L1), cytotoxic T-lymphocyte-associated protein 4, and any combination thereof.

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in other aspects the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substantially free” means that the sample comprising EVs, e.g., exosomes, comprise less than 10% of macromolecules by mass/volume (m/v) percentage concentration. Some fractions can contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein, the term “macromolecule” means nucleic acids, contaminant proteins, lipids, carbohydrates, metabolites, or a combination thereof.

As used herein, the term “conventional exosome protein” means a protein previously known to be enriched in exosomes, including but is not limited to CD9, CD63, CD81, PDGFR, GPI anchor proteins, lactadherin LAMP2, and LAMP2B, a fragment thereof, or a peptide that binds thereto.

“Administering,” as used herein, means to give a composition comprising an EV, e.g., exosome, disclosed herein to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. EVs, e.g., exosomes can be administered as part of a pharmaceutical composition comprising at least one excipient.

An “immune response,” as used herein, refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly an immune response can comprise a humoral immune response (e.g., mediated by B-cells), cellular immune response (e.g., mediated by T cells), or both humoral and cellular immune responses. In some aspects, an immune response is an “inhibitory” immune response. An inhibitory immune response is an immune response that blocks or diminishes the effects of a stimulus (e.g., antigen). In certain aspects, the inhibitory immune response comprises the production of inhibitory antibodies against the stimulus. In some aspects, an immune response is a “stimulatory” immune response. A stimulatory immune response is an immune response that results in the generation of effectors cells (e.g., cytotoxic T lymphocytes) that can destroy and clear a target antigen (e.g., tumor antigen or viruses).

As used herein, the term “immune cells” refers to any cells of the immune system that are involved in mediating an immune response. Non-limiting examples of immune cells include a T lymphocyte, B lymphocyte, natural killer (NK) cell, macrophage, eosinophil, mast cell, dendritic cell, neutrophil, or combination thereof. In some aspects, an immune cell is a dendritic cell. In certain aspects, a dendritic cell comprises a plasmacytoid dendritic cell (pDC), a conventional dendritic cell 1 (cDC1), a conventional dendritic cell 2 (cDC2), or any combination thereof. Accordingly, in certain aspects, an immune cell that an EV disclosed herein (e.g., exosomes) can specifically target includes a conventional dendritic cell 1 (cDC1) and/or plasmacytoid dendritic cells (pDC).

As used herein, the term “dendritic cells” or “DCs” refers to a class of bone-marrow-derived immune cells that are capable of processing extracellular and intracellular proteins and to present antigens in the context of MHC molecules to prime naïve T cells. In some aspects, dendritic cells can be divided into three subsets: (i) conventional dendritic cell 1 (cDC1), (ii) conventional dendritic cell 2 (cDC2), and (iii) plasmacytoid dendritic cell (pDC). In certain aspects, the different DC subsets can be distinguished based on their phenotypic expression. For example, in some aspects, human cDC1 cells are CD1c⁻ and CD141⁺. In some aspects, human cDC2 cells are CDlc⁺ and CD141⁻. In some aspects, human pDC cells are CD123⁺. In some aspects, mouse cDC1 cells are XCR1⁺, Clec9a⁺, and Sirpa⁻. In some aspects, mouse cDC2 cells are CD8⁺, CD11b⁺, Sirpa⁺, XCR1⁻, and CD1c,b⁺. In some aspects, mouse pDC cells are CD137⁺, XCR1⁻, and Sirpa⁻. Other phenotypic markers for distinguishing the different DC subsets are known in the art. See, e.g., Collin et al., Immunology 154(1): 3-20 (2018). In some aspects, the different DC subsets can be distinguished based on their functional properties. For example, in certain aspects, pDCs produce large amounts of IFN-α, while cDC1s and cDC2s produce inflammatory cytokines, such as IL-12, IL-6, and TNF-α. Other methods of distinguishing the different DC subsets are known in the art. See, e.g., U.S. Pat. Nos. 8,426,565 B2 and 9,988,431, each of which is herein incorporated by reference in its entirety.

“Treat,” “treatment,” or “treating,” as used herein, refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

“Prevent” or “preventing,” as used herein, refers to decreasing or reducing the occurrence or severity of a particular outcome. In some aspects, preventing an outcome is achieved through prophylactic treatment.

### II. Extracellular Vesicles, e.g., Exosomes

Disclosed herein are modified EVs, e.g., exosomes, capable of regulating the immune system of a subject. The EVs, e.g., exosomes, useful in the present disclosure have been engineered to express a targeting moiety (i.e., exogenous targeting moiety) that allows the EVs (e.g., exosomes) to target a specific population of immune cells (e.g., dendritic cells). In certain aspects, the targeting moiety binds to a marker (e.g., those disclosed herein) that is expressed on the immune cells. In further aspects, the marker is expressed only on the immune cells. In still further aspects, the EVs of the present disclosure (e.g., exosomes) can comprise multiple (e.g., two or more) targeting moieties. In some aspects, the multiple targeting moieties bind to the same marker. In other aspects, the multiple targeting moieties bind to different markers.

In some aspects, an EV (e.g., exosome) can further comprise one or more additional exogenous biologically active molecules, e.g., an antigen, adjuvant, and/or immune modulator. Accordingly, in certain aspects, an EV disclosed herein (e.g., exosome) comprises (i) a targeting moiety (e.g., disclosed herein) and (ii) an antigen. In some aspects, an EV (e.g., exosome) comprises (i) a targeting moiety and (ii) an adjuvant. In some aspects, an EV (e.g., exosome) comprises (i) a targeting moiety and (ii) an immune modulator. In further aspects, an EV disclosed herein (e.g., exosome) comprises a (i) a targeting moiety, (ii) an antigen, (iii) an adjuvant, and (iv) an immune modulator.

As described supra, EVs, e.g., exosomes, described herein are extracellular vesicles with a diameter between about 20-300 nm. In certain aspects, an EV, e.g., exosome, of the present disclosure has a diameter between about 20-290 nm, 20-280 nm, 20-270 nm, 20-260 nm, 20-250 nm, 20-240 nm, 20-230 nm, 20-220 nm, 20-210 nm, 20-200 nm, 20-190 nm, 20-180 nm, 20-170 nm, 20-160 nm, 20-150 nm, 20-140 nm, 20-130 nm, 20-120 nm, 20-110 nm, 20-100 nm, 20-90 nm, 20-80 nm, 20-70 nm, 20-60 nm, 20-50 nm, 20-40 nm, 20-30 nm, 30-300 nm, 30-290 nm, 30-280 nm, 30-270 nm, 30-260 nm, 30-250 nm, 30-240 nm, 30-230 nm, 30-220 nm, 30-210 nm, 30-200 nm, 30-190 nm, 30-180 nm, 30-170 nm, 30-160 nm, 30-150 nm, 30-140 nm, 30-130 nm, 30-120 nm, 30-110 nm, 30-100 nm, 30-90 nm, 30-80 nm, 30-70 nm, 30-60 nm, 30-50 nm, 30-40 nm, 40-300 nm, 40-290 nm, 40-280 nm, 40-270 nm, 40-260 nm, 40-250 nm, 40-240 nm, 40-230 nm, 40-220 nm, 40-210 nm, 40-200 nm, 40-190 nm, 40-180 nm, 40-170 nm, 40-160 nm, 40-150 nm, 40-140 nm, 40-130 nm, 40-120 nm, 40-110 nm, 40-100 nm, 40-90 nm, 40-80 nm, 40-70 nm, 40-60 nm, 40-50 nm, 50-300 nm, 50-290 nm, 50-280 nm, 50-270 nm, 50-260 nm, 50-250 nm, 50-240 nm, 50-230 nm, 50-220 nm, 50-210 nm, 50-200 nm, 50-190 nm, 50-180 nm, 50-170 nm, 50-160 nm, 50-150 nm, 50-140 nm, 50-130 nm, 50-120 nm, 50-110 nm, 50-100 nm, 50-90 nm, 50-80 nm, 50-70 nm, 50-60 nm, 60-300 nm, 60-290 nm, 60-280 nm, 60-270 nm, 60-260 nm, 60-250 nm, 60-240 nm, 60-230 nm, 60-220 nm, 60-210 nm, 60-200 nm, 60-190 nm, 60-180 nm, 60-170 nm, 60-160 nm, 60-150 nm, 60-140 nm, 60-130 nm, 60-120 nm, 60-110 nm, 60-100 nm, 60-90 nm, 60-80 nm, 60-70 nm, 70-300 nm, 70-290 nm, 70-280 nm, 70-270 nm, 70-260 nm, 70-250 nm, 70-240 nm, 70-230 nm, 70-220 nm, 70-210 nm, 70-200 nm, 70-190 nm, 70-180 nm, 70-170 nm, 70-160 nm, 70-150 nm, 70-140 nm, 70-130 nm, 70-120 nm, 70-110 nm, 70-100 nm, 70-90 nm, 70-80 nm, 80-300 nm, 80-290 nm, 80-280 nm, 80-270 nm, 80-260 nm, 80-250 nm, 80-240 nm, 80-230 nm, 80-220 nm, 80-210 nm, 80-200 nm, 80-190 nm, 80-180 nm, 80-170 nm, 80-160 nm, 80-150 nm, 80-140 nm, 80-130 nm, 80-120 nm, 80-110 nm, 80-100 nm, 80-90 nm, 90-300 nm, 90-290 nm, 90-280 nm, 90-270 nm, 90-260 nm, 90-250 nm, 90-240 nm, 90-230 nm, 90-220 nm, 90-210 nm, 90-200 nm, 90-190 nm, 90-180 nm, 90-170 nm, 90-160 nm, 90-150 nm, 90-140 nm, 90-130 nm, 90-120 nm, 90-110 nm, 90-100 nm, 100-300 nm, 110-290 nm, 120-280 nm, 130-270 nm, 140-260 nm, 150-250 nm, 160-240 nm, 170-230 nm, 180-220 nm, or 190-210 nm. The size of the EV, e.g., exosome, described herein can be measured according to methods described, infra.

In some aspects, an EV, e.g., exosome, of the present disclosure comprises a bi-lipid membrane (“EV, e.g., exosome, membrane”), comprising an interior surface and an exterior surface. In certain aspects, the interior surface faces the inner core (i.e., lumen) of the EV, e.g., exosome. In certain aspects, the exterior surface can be in contact with the endosome, the multivesicular bodies, or the membrane/cytoplasm of a producer cell or a target cell.

In some aspects, the EV, e.g., exosome, membrane comprises lipids and fatty acids. In some aspects, the EV, e.g., exosome, membrane comprises phospholipids, glycolipids, fatty acids, sphingolipids, phosphoglycerides, sterols, cholesterols, and phosphatidylserines.

In some aspects, the EV, e.g., exosome, membrane comprises an inner leaflet and an outer leaflet. The composition of the inner and outer leaflet can be determined by transbilayer distribution assays known in the art, see, e.g., Kuypers et al., Biohim Biophys Acta 1985 819:170. In some aspects, the composition of the outer leaflet is between approximately 70-90% choline phospholipids, between approximately 0-15% acidic phospholipids, and between approximately 5-30% phosphatidylethanolamine. In some aspects, the composition of the inner leaflet is between approximately 15-40% choline phospholipids, between approximately 10-50% acidic phospholipids, and between approximately 30-60% phosphatidylethanolamine.

In some aspects, the EV, e.g., exosome, membrane comprises one or more polysaccharide, such as glycan.

In some aspects, the EV, e.g., exosome, membrane further comprises one or more scaffold moieties, which are capable of anchoring a targeting moiety disclosed herein (e.g., on the exterior surface of the EV). Accordingly, in certain aspects, an EV disclosed herein (e.g., exosome), comprises a targeting moiety and a scaffold moiety, wherein the scaffold moiety anchors or links the targeting moiety to the EV (e.g., on the exterior surface of the EV). In some aspects, at least one of the additional exogenous biologically active molecules (e.g., antigen, adjuvant, or immune modulator) that can be expressed in the EVs disclosed herein (e.g., exosomes) is also anchored or linked to the EV via a scaffold moiety (e.g., either on the exterior surface or on the luminal surface). or any other exogenous biologically active molecules disclosed herein. In some aspects, each of the additional exogenous biologically active molecules expressed in an EV (e.g., antigen, adjuvant, or immune modulator) is anchored or linked to the EV via a scaffold moiety. In certain aspects, scaffold moieties are polypeptides (“exosome proteins”). In other aspects, scaffold moieties are non-polypeptide moieties. In some aspects, exosome proteins include various membrane proteins, such as transmembrane proteins, integral proteins and peripheral proteins, enriched on the exosome membranes. They can include various CD proteins, transporters, integrins, lectins, and cadherins. In certain aspects, a scaffold moiety (e.g., exosome protein) comprises Scaffold X. In other aspects, a scaffold moiety (e.g., exosome protein) comprises Scaffold Y. In further aspects, a scaffold moiety (e.g., exosome protein) comprises both a Scaffold X and a Scaffold Y. Additional disclosure relating to the scaffold moieties that can be used with the present disclosure are provided throughout the present disclosure.

### Targeting Moieties

An EV (e.g., exosome) disclosed herein have been engineered or modified to target a specific cell of interest (e.g., dendritic cells). In some aspects, an EV (e.g., exosome) comprises a targeting moiety that specifically binds to a marker (or target molecule) expressed on a cell or a population of cells. In certain aspects, the marker is expressed on multiple cell types, e.g., all antigen-presenting cells (e.g., dendritic cells, macrophages, and B lymphocytes). In other aspects, the marker is expressed only on a specific population of cells (e.g., dendritic cells).

In some aspects, a targeting moiety of the present disclosure specifically binds to a marker for a dendritic cell. In certain aspects, the marker is expressed only on dendritic cells. In some aspects, dendritic cells comprise a plasmacytoid dendritic cell (pDC), a myeloid/conventional dendritic cell 1 (cDC1), a myeloid/conventional dendritic cell 2 (cDC2), or any combination thereof. In some aspects, a marker comprises a C-type lectin domain family 9 member A (Clec9a) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), or any combination thereof. In some aspects, a marker useful for the present disclosure comprises a C-type lectin like domain. In certain aspects, a marker is Clec9a and the dendritic cell is cDC1.

In some aspects, a targeting moiety disclosed herein binds to human Clec9a protein or a fragment thereof. Sequences for human Clec9a protein are known in the art. For instance, the amino acid sequence for human Clec9a consist of 241 amino acids and can be found under publicly available UniProt Accession Numbers Q6UXN8 (canonical sequence; SEQ ID NO: 370) and B0ZBM2, each of which is herein incorporated by reference in its entirety. A natural variant of human Clec9a protein exists and contains the following amino acid substitution: A107G.

In some aspects, a targeting moiety disclosed herein binds to mouse Clec9a protein or a fragment thereof. Sequences for mouse Clec9a protein is also known in the art. The amino acid sequence for mouse Clec9A can be found under publicly available UniProt Accession Number Q8BRU4 (canonical sequence, SEQ ID NO: 371), B0ZBM3, BaZBM5, B0ZBM6, B0ZBM7, Q5M8M6, and Q8CBB4, each of which is herein incorporated by reference in its entirety. Variants of mouse Clec9a protein resulting from alternative splicing are known in the art. The sequence for Clec9a isoform 2 (Uniprot identifier: Q8BRU4-2) differs from the canonical sequence (SEQ ID NO: 372) as follows: 31-58: GAWCVVTMISCVVCMGLLATSIFLGIKF→V; 105-105: G→GTDASTGPVLLTSPQMVPQTLDSKETG. The sequence for Clec9a isoform 3 (Uniprot identifier: Q8BRU4-3) differs from the canonical sequence (SEQ ID NO: 373) as follows: 156-173: EFISSIGKLKGGNKYWVG→SRWNQWILVLGRWLFSSL; and 174-238: Missing. The sequence for Clec9a isoform 4 (Uniprot identifier: Q8BRU4-4) differs from the canonical sequence (SEQ ID NO: 374) as follows: 105-105: G→GTDASTGPVLLTSPQMVPQTLDSKETG. The sequence for Clec9a isoform 5 (Uniprot identifier: Q8BRU4-5) differs from the canonical sequence (SEQ ID NO: 375) as follows: 106-147: SDCSPCPHNW . . . SCLKEGASLF→CQQKDSDQPA . . . VRRRHLDPAS; and 148-238: Missing. Therefore, a targeting moiety disclosed herein can bind to any such variants of mouse Clec9a.

In some aspects, a targeting moiety disclosed herein can bind to both human and mouse Clec9a, including any variants thereof. In some aspects, a targeting moiety of the present disclosure can bind to Clec9a from other species, including but not limited to chimpanzee, rhesus monkey, dog, cow, horse, or rat. Sequences for such Clec9a protein are known in the art. See, e.g., U.S. Pat. No. 8,426,565 B2, which is herein incorporated by reference in its entirety.

In some aspects, a targeting moiety disclosed herein can allow for greater uptake of an EV (e.g., exosome) by a cell expressing a marker specific for the targeting moiety (e.g., cDC1). In certain aspects, the uptake of an EV is increased by at least about 5%, at least about 10%, at least about 20%, at least about 30%, at least about 40%, at least about 50%, at least about 60%, at least about 70%, at least about 80%, at least about 90%, at least about 100% or more, compared to a reference. In some aspects, a reference comprises an EV (e.g., exosome) that does not express a targeting moiety disclosed herein.

In some aspects, the increased uptake of an EV (e.g., exosome) disclosed herein can allow for greater immune response. Accordingly, in certain aspects, an EV (e.g., exosome) expressing a targeting moiety disclosed herein can increase an immune response (e.g., against a tumor antigen loaded onto the exosome) by at least about 5%, at least about 10%, at least about 20%, at least about 30%, at least about 40%, at least about 50%, at least about 60%, at least about 70%, at least about 80%, at least about 90%, at least about 100% or more, compared to a reference. In some aspects, a reference comprises an EV (e.g., exosome) that does not express a targeting moiety disclosed herein. In certain aspects, an immune response is mediated by T cells (e.g., CD8+ T cells or CD4+ T cells) and/or B cells.

As described supra, a targeting moiety disclosed herein can comprise a peptide, an antibody or an antigen binding fragment thereof, a chemical compound, or any combination thereof. In some aspects, the targeting moiety is a peptide that can specifically bind to Clec9a. See, e.g., Yan et al., Oncotarget 7(26): 40437-40450 (2016). For example, in certain aspects, the peptide comprises a soluble fragment of Clec9a. A non-limiting example of such a peptide is described in U.S. Pat. No. 9,988,431 B2, which is herein incorporated by reference in its entirety. In certain aspects, the peptide comprises a ligand (natural or synthetic) of Clec9a, such as those described in Ahrens et al., Immunity 36(4): 635-45 (2012); and Zhang et al., Immunity 36(4): 646-57 (2012). A non-limiting example of a peptide comprising a Clec9a ligand is described in International Publ. No. WO 2013/053008 A2, which is herein incorporated by reference in its entirety.

In some aspects, the targeting moiety is an antibody or an antigen binding fragment thereof. In certain aspects, a targeting moiety is a single-chain Fv antibody fragment. In certain aspects, a targeting moiety is a single-chain F(ab) antibody fragment. In certain aspects, a targeting moiety is a nanobody. In certain aspects, a targeting moiety is a monobody. Non-limiting examples of Clec9a binding agents, which can be used as a targeting moiety described herein, are provided in U.S. Publ. No. 2013/0273150 A1; U.S. Pat. Nos. 8,426,565 B2; 8,580,266 B2; International Publ. Nos. WO 2017/134301 A1; WO 2013/053008 A2; WO 2019/032662 A1; WO 2011/044452 A2, each of which is herein incorporated by reference in its entirety.

In some aspects, an EV (e.g., exosome) disclosed herein comprises one or more (e.g., 2, 3, 4, 5, or more) targeting moieties. In certain aspects, the one or more targeting moieties are expressed in combination with other exogenous biologically active molecules disclosed herein (e.g., therapeutic molecule, adjuvant, or immune modulator). In some aspects, the one or more targeting moieties can be expressed on the exterior surface of the EV, e.g., exosome. Accordingly, in certain aspects, the one or more targeting moieties are linked to a scaffold moiety (e.g., Scaffold X) on the exterior surface of the EV, e.g., exosome. When the one or more targeting moieties are expressed in combination with other exogenous biologically active molecules (e.g., therapeutic molecule, adjuvant, or immune modulator), the other exogenous biologically active molecules can be expressed on the surface (e.g., exterior surface or luminal surface) or in the lumen of the EV, e.g., exosome.

### Therapeutic Molecules

In some aspects, an EV (e.g., exosome) disclosed herein has been engineered or modified to deliver one or more (e.g., two, three, four, five or more) therapeutic molecules to a target. In certain aspects, a therapeutic molecule comprises an antigen. According, in certain aspects, an EV (e.g., exosome) disclosed herein comprises a targeting moiety and an antigen.

In some aspects, an antigen that can be delivered using an EV (e.g., exosome) disclosed herein comprises a tumor antigen. Non-limiting examples of tumor antigens include: alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen (CTA), MART-1 gp100, TNF-related apoptosis-inducing ligand, Brachyury (preferentially expressed antigen in melanoma (PRAME)), or combinations thereof. In further aspects, an antigen can comprise a neoantigen. As used herein, the term “neoantigen,” refers to antigens encoded by tumor-specific mutated genes. In some aspects, the antigen is derived from a bacterium, a virus, fungus, protozoa, or any combination thereof. In some aspects, the antigen is derived from an oncogenic virus. In further aspects, the antigen is derived from a group comprising: a Human Gamma herpes virus 4 (Epstein Barr virus), influenza A virus, influenza B virus, cytomegalovirus, Staphylococcus aureus, Mycobacterium tuberculosis, Chlamydia trachomatis, HIV-1, HIV-2, corona viruses (e.g., MERS-CoV and SARS CoV), filoviruses (e.g., Marburg and Ebola), Streptococcus pyogenes, Streptococcus pneumoniae, Plasmodia species (e.g., vivax and falciparum), Chikungunya virus, Human Papilloma virus (HPV), Hepatitis B, Hepatitis C, human herpes virus 8, herpes simplex virus 2 (HSV2), Klebsiella sp., Pseudomonas aeruginosa, Enterococcus sp., Proteus sp., Enterobacter sp., Actinobacter sp., coagulase-negative staphylococci (CoNS), Mycoplasma sp., or combinations thereof.

In some aspects, a therapeutic molecule comprises an immunosuppressive agent. Accordingly, in certain aspects, an EV (e.g., exosome) disclosed herein comprises a targeting moiety and an immunosuppressive agent.

Non-limiting examples of other suitable therapeutic molecules include pharmacologically active drugs and genetically active molecules, including antineoplastic agents, anti-inflammatory agents, hormones or hormone antagonists, ion channel modifiers, and neuroactive agents. Examples of suitable payloads of therapeutic agents include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Suitable payloads further include toxins, and biological and chemical warfare agents, for example see Somani, S. M. (ed.), Chemical Warfare Agents, Academic Press, New York (1992)).

In certain aspects, an EV (e.g., exosomes) disclosed herein have been engineered or modified to comprise two or more therapeutic molecules (e.g., antigen or immunosuppressive agent), a first therapeutic molecule and a second therapeutic molecule (e.g., in addition to a targeting moiety disclosed herein). In some aspects, the first therapeutic molecule is linked to a first Scaffold Y on the luminal surface of the EV, e.g., exosome, and the second therapeutic molecule is linked to a second Scaffold Y on the luminal surface of the EV, e.g., exosome. In some aspects, the first therapeutic molecule is linked to a Scaffold Y on the luminal surface of the EV, e.g., exosome, and the second therapeutic molecule is in the lumen of the EV, e.g., exosome, not linked to any scaffold moiety. In some aspects, the first therapeutic molecule is in the lumen of the EV, e.g., exosome, not linked to any scaffold moiety, and the second therapeutic molecule is linked to a Scaffold Y on the luminal surface of the EV, e.g., exosome. In some aspects, the first therapeutic molecule is linked to a Scaffold Y on the luminal surface of the EV, e.g., exosome, and the second therapeutic molecule is linked to a Scaffold X on the exterior surface of the EV, e.g., exosome. In some aspects, the first therapeutic molecule is in the lumen of the EV, e.g., exosome, not linked to any scaffold moiety, and the second therapeutic molecule is linked to a Scaffold X on the exterior surface of the EV, e.g., exosome. In some aspects, the first therapeutic molecule is linked to a Scaffold Y on the luminal surface of the EV, e.g., exosome, and the second therapeutic molecule is linked to a Scaffold X on the luminal surface of the EV, e.g., exosome. In some aspects, the first therapeutic molecule is in the lumen of the EV, e.g., exosome, not linked to any scaffold moiety, and the second therapeutic molecule is linked to a Scaffold X on the luminal surface of the EV, e.g., exosome. In some aspects, the first therapeutic molecule is linked to a Scaffold X on the luminal surface of the EV, e.g., exosome, and the second therapeutic molecule is linked to the Scaffold X on the exterior surface of the EV, e.g., exosome. In some aspects, the first therapeutic molecule is linked to a first Scaffold X on the exterior surface of the EV, e.g., exosome, and the second therapeutic molecule is linked to a second Scaffold X on the exterior surface of the EV, e.g., exosome. In some aspects, the first therapeutic molecule is linked to a Scaffold X on the exterior surface of the EV, e.g., exosome, and the second therapeutic molecule is linked to a Scaffold Y on the luminal surface of the EV, e.g., exosome. In some aspects, the first therapeutic molecule is linked to a Scaffold X on the exterior surface of the EV, e.g., exosome, and the second therapeutic molecule is in the lumen of the EV, e.g., exosome, not linked to any scaffold moiety. In some aspects, the first therapeutic molecule is linked to a Scaffold X on the exterior surface of the EV, e.g., exosome, and the second therapeutic molecule is linked to the Scaffold X on the luminal surface of the EV, e.g., exosome. In some aspects, the first therapeutic molecule is linked to a first Scaffold X on the luminal surface of the EV, e.g., exosome, and the second therapeutic molecule is linked to a second Scaffold X on the luminal surface of the EV, e.g., exosome. In some aspects, the first therapeutic molecule is linked to a Scaffold X on the luminal surface of the EV, e.g., exosome, and the second therapeutic molecule is linked to a Scaffold Y on the luminal surface of the EV, e.g., exosome. In some aspects, the first therapeutic molecule is linked to a Scaffold X on the luminal surface of the EV, e.g., exosome, and the second therapeutic molecule is in the lumen of the EV, e.g., exosome, not linked to any scaffold moiety. In some aspects, the first therapeutic molecule is linked to a first Scaffold X on the exterior surface of the EV, e.g., exosome, and the second therapeutic molecule is linked to a second Scaffold X on the luminal surface of the EV, e.g., exosome. In some aspects, the first therapeutic molecule is linked to a first Scaffold X on the luminal surface of the EV, e.g., exosome, and the second therapeutic molecule is linked to a second Scaffold X on the exterior surface of the EV, e.g., exosome. In some aspects, the first therapeutic molecule is in the lumen of the EV, e.g., exosome, not linked to any scaffold moiety, and the second therapeutic molecule is in the lumen of the EV, e.g., exosome, not linked to any scaffold moiety.

In some aspects, a therapeutic molecule comprises a self-antigen. As used herein, the term “self-antigen” refers to an antigen that is expressed by a host cell or tissue. Under normal healthy state, such antigens are recognized by the body as self and do not elicit an immune response. However, under certain diseased conditions, a body's own immune system can recognize self-antigens as foreign and mount an immune response against them, resulting in autoimmunity. In certain aspects, EVs, e.g., exosomes, of the present disclosure can comprise a self-antigen (i.e., the self (germline) protein to which T cell responses have been induced and resulted in autoimmunity). Such EVs, e.g., exosomes, can be used to target the autoreactive T cells and suppress their activity. Non-limiting examples of self-antigens (including the associated disease or disorder) include: beta-cell proteins (type I diabetes), myelin oligodendrocyte glycoprotein (MOG, multiple sclerosis), synovial proteins (rheumatoid arthritis), or combinations thereof.

In some aspects, the therapeutic molecule comprises an antibody or antigen-binding fragment thereof. In some aspects, the therapeutic molecule comprises at least 2, at least 3, at least 4, or at least 5 antibodies or antigen-binding fragments thereof. In some aspects, the antibody or antigen-binding fragment thereof comprises a scFv, scFab, scFab-Fc, nanobody, or any combination thereof. In some aspects, the antibody or antigen-binding fragment thereof comprises an agonist antibody, blocking antibody, a targeting antibody, a fragment thereof, or a combination thereof. In some aspects, the agonist antibody is a CD40L agonist. In some aspects, the blocking antibody binds a target protein selected from programmed death 1 (PD-1), programmed death ligand 1 (PD-L1), cytotoxic T-lymphocyte-associated protein 4, and any combination thereof. In some aspects, the EV, e.g., exosome, comprises an anti-IL12 antibody or an antigen-binding fragment thereof and an anti-CD40L antibody or antigen-binding fragment thereof.

### Adjuvants

As described supra, EVs, e.g., exosomes, of the present disclosure can comprise one or more exogenous biologically active molecules. In some aspects, an exogenous biologically active molecule that can be expressed in an EV (e.g., exosome) is an adjuvant. Accordingly, in certain aspects, an EV (e.g., exosome) disclosed herein comprises a targeting moiety and an adjuvant. In some aspects, EVs (e.g., exosome) disclosed herein comprises two, three, four, five or more different adjuvants. As used herein, the term “adjuvant” refers to any substance that enhances the therapeutic effect of the payload (e.g., increasing an immune response to the antigen). Accordingly, EVs, e.g., exosomes, described herein are capable of increasing an immune response to an antigen by at least about 5%, at least about 10%, at least about 20%, at least about 30%, at least about 40%, at least about 50%, at least about 60%, at least about 70%, at least about 80%, at least about 90%, at least about 100% or more, compared to a reference (e.g., corresponding EV without the adjuvant or a non-EV delivery vehicle comprising an antigen and adjuvant). Non-limiting examples of adjuvants include: Stimulator of Interferon Genes (STING) agonist, a toll-like receptor (TLR) agonist, an inflammatory mediator, and combinations thereof.

In certain aspects, the present disclosure is directed to modified or engineered EVs comprising two or more exogenous biologically active molecules, wherein the two or more exogenous biologically active molecules are adjuvants, a first adjuvant and a second adjuvant (e.g., in addition to a targeting moiety disclosed herein). In some aspects, the first adjuvant is linked to a first Scaffold Y on the luminal surface of the EV, e.g., exosome, and the second adjuvant is linked to a second Scaffold Y on the luminal surface of the EV, e.g., exosome. In some aspects, the first adjuvant is linked to a Scaffold Y on the luminal surface of the EV, e.g., exosome, and the second adjuvant is in the lumen of the EV, e.g., exosome, not linked to any scaffold moiety. In some aspects, the first adjuvant is in the lumen of the EV, e.g., exosome, not linked to any scaffold moiety, and the second adjuvant is linked to a Scaffold Y on the luminal surface of the EV, e.g., exosome. In some aspects, the first adjuvant is linked to a Scaffold Y on the luminal surface of the EV, e.g., exosome, and the second adjuvant is linked to a Scaffold X on the exterior surface of the EV, e.g., exosome. In some aspects, the first adjuvant is in the lumen of the EV, e.g., exosome, not linked to any scaffold moiety, and the second adjuvant is linked to a Scaffold X on the exterior surface of the EV, e.g., exosome. In some aspects, the first adjuvant is linked to a Scaffold Y on the luminal surface of the EV, e.g., exosome, and the second adjuvant is linked to a Scaffold X on the luminal surface of the EV, e.g., exosome. In some aspects, the first adjuvant is in the lumen of the EV, e.g., exosome, not linked to any scaffold moiety, and the second adjuvant is linked to a Scaffold X on the luminal surface of the EV, e.g., exosome. In some aspects, the first adjuvant is linked to a Scaffold X on the luminal surface of the EV, e.g., exosome, and the second adjuvant is linked to the Scaffold X on the exterior surface of the EV, e.g., exosome. In some aspects, the first adjuvant is linked to a first Scaffold X on the exterior surface of the EV, e.g., exosome, and the second adjuvant is linked to a second Scaffold X on the exterior surface of the EV, e.g., exosome. In some aspects, the first adjuvant is linked to a Scaffold X on the exterior surface of the EV, e.g., exosome, and the second adjuvant is linked to a Scaffold Y on the luminal surface of the EV, e.g., exosome. In some aspects, the first adjuvant is linked to a Scaffold X on the exterior surface of the EV, e.g., exosome, and the second adjuvant is in the lumen of the EV, e.g., exosome, not linked to any scaffold moiety. In some aspects, the first adjuvant is linked to a Scaffold X on the exterior surface of the EV, e.g., exosome, and the second adjuvant is linked to the Scaffold X on the luminal surface of the EV, e.g., exosome. In some aspects, the first adjuvant is linked to a first Scaffold X on the luminal surface of the EV, e.g., exosome, and the second adjuvant is linked to a second Scaffold X on the luminal surface of the EV, e.g., exosome. In some aspects, the first adjuvant is linked to a Scaffold X on the luminal surface of the EV, e.g., exosome, and the second adjuvant is linked to a Scaffold Y on the luminal surface of the EV, e.g., exosome. In some aspects, the first adjuvant is linked to a Scaffold X on the luminal surface of the EV, e.g., exosome, and the second adjuvant is in the lumen of the EV, e.g., exosome, not linked to any scaffold moiety. In some aspects, the first adjuvant is linked to a first Scaffold X on the exterior surface of the EV, e.g., exosome, and the second adjuvant is linked to a second Scaffold X on the luminal surface of the EV, e.g., exosome. In some aspects, the first adjuvant is linked to a first Scaffold X on the luminal surface of the EV, e.g., exosome, and the second adjuvant is linked to a second Scaffold X on the exterior surface of the EV, e.g., exosome. In some aspects, the first adjuvant is in the lumen of the EV, e.g., exosome, not linked to any scaffold moiety, and the second adjuvant is in the lumen of the EV, e.g., exosome, not linked to any scaffold moiety.

In some aspects, an adjuvant useful for the present disclosure induces the activation of a cytosolic pattern recognition receptor. Non-limiting examples of cytosolic pattern recognition receptor includes: stimulator of interferon genes (STING), retinoic acid-inducible gene I (RIG-1), Melanoma Differentiation-Associated protein 5 (MDA5), Nucleotide-binding oligomerization domain, Leucine rich Repeat and Pyrin domain containing (NLRP), inflammasomes, or combinations thereof. In certain aspects, an adjuvant is a STING agonist. Stimulator of Interferon Genes (STING) is a cytosolic sensor of cyclic dinucleotides that is typically produced by bacteria. Upon activation, it leads to the production of type I interferons and initiates an immune response. In certain aspects, the STING agonist comprises a cyclic dinucleotide STING agonist or a non-cyclic dinucleotide STING agonist.

Cyclic purine dinucleotides such as, but not limited to, cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, are known to stimulate or enhance an immune or inflammation response in a patient. The CDNs can have 2′2′, 2′3′, 2′5′, 3′3′, or 3′5′ bonds linking the cyclic dinucleotides, or any combination thereof.

Cyclic purine dinucleotides can be modified via standard organic chemistry techniques to produce analogues of purine dinucleotides. Suitable purine dinucleotides include, but are not limited to, adenine, guanine, inosine, hypoxanthine, xanthine, isoguanine, or any other appropriate purine dinucleotide known in the art. The cyclic dinucleotides can be modified analogues. Any suitable modification known in the art can be used, including, but not limited to, phosphorothioate, biphosphorothioate, fluorinate, and difluorinate modifications.

Non cyclic dinucleotide agonists can also be used, such as 5,6-Dimethylxanthenone-4-acetic acid (DMXAA), or any other non-cyclic dinucleotide agonist known in the art.

Non-limiting examples of STING agonists that can be used with the present disclosure include: DMXAA, STING agonist-1, ML RR-S2 CDA, ML RR-S2c-di-GMP, ML-RR-S2 cGAMP, 2′3′-c-di-AM(PS)2, 2′3′-cGAMP, 2′3′-cGAMPdFHS, 3′3′-cGAMP, 3′3′-cGAMPdFSH, cAIMP, cAIM(PS)2, 3′3′-cAIMP, 3′3′-cAIMIPdFSH, 2′2′-cGAMP, 2′3′-cGAM(PS)2, 3′3′-cGAMP, and combinations thereof. Non-limiting examples of the STING agonists can be found at U.S. Pat. No. 9,695,212, WO 2014/189805 A1, WO 2014/179335 A1, WO 2018/100558 A1, U.S. Pat. No. 10,011,630 B2, WO 2017/027646 A1, WO 2017/161349 A1, and WO 2016/096174 A1, each of which is incorporated by reference in its entirety.

In some aspects, the STING agonist useful for the present disclosure comprises a compound or a pharmaceutically acceptable salt thereof disclosed in WO 2016/096174, WO 2016/096174A1, WO 2014/093936, WO 2014/189805, WO 2015/077354, the content of which is incorporated herein by reference in its entirety. See also Cell reports 11, 1018-1030 (2015).

In some aspects, the STING agonist useful for the present disclosure comprises c-di-AMP, c-di-GMP, c-di-IMP, c-AMP-GMP, c-AMP-IMP, and c-GMP-IMP, described in WO 2013/185052 and Sci. Transl. Med. 283,283ra52 (2015), which are incorporated herein by reference in their entireties.

In some aspects, the STING agonist useful for the present disclosure comprises a compound or a pharmaceutically acceptable salt thereof disclosed in WO 2014/189806, WO 2015/185565, WO 2014/179760, WO 2014/179335, WO 2015/017652, WO 2016/096577, WO 2016/120305, WO 2016/145102, WO 2017/027646, WO 2017/075477, WO 2017/027645, WO 2018/100558, WO 2017/175147, or WO 2017/175156, each content of which is incorporated herein by reference in its entirety.

In some aspects, the STING agonist useful for the present disclosure is CL606, CL611, CL602, CL655, CL604, CL609, CL614, CL656, CL647, CL626, CL629, CL603, CL632, CL633, CL659, or a pharmaceutically acceptable salt thereof. In some aspects, the STING agonist useful for the present disclosure is CL606 or a pharmaceutically acceptable salt thereof. In some aspects, the STING agonist useful for the present disclosure is CL611 or a pharmaceutically acceptable salt thereof. In some aspects, the STING agonist useful for the present disclosure is CL602 or a pharmaceutically acceptable salt thereof. In some aspects, the STING agonist useful for the present disclosure is CL655 or a pharmaceutically acceptable salt thereof. In some aspects, the STING agonist useful for the present disclosure is CL604 or a pharmaceutically acceptable salt thereof. In some aspects, the STING agonist useful for the present disclosure is CL609 or a pharmaceutically acceptable salt thereof. In some aspects, the STING agonist useful for the present disclosure is CL614 or a pharmaceutically acceptable salt thereof. In some aspects, the STING agonist useful for the present disclosure is CL656 or a pharmaceutically acceptable salt thereof. In some aspects, the STING agonist useful for the present disclosure is CL647 or a pharmaceutically acceptable salt thereof. In some aspects, the STING agonist useful for the present disclosure is CL626 or a pharmaceutically acceptable salt thereof. In some aspects, the STING agonist useful for the present disclosure is CL629 or a pharmaceutically acceptable salt thereof. In some aspects, the STING agonist useful for the present disclosure is CL603 or a pharmaceutically acceptable salt thereof. In some aspects, the STING agonist useful for the present disclosure is CL632 or a pharmaceutically acceptable salt thereof. In some aspects, the STING agonist useful for the present disclosure is CL633 or a pharmaceutically acceptable salt thereof. In some aspects, the STING agonist useful for the present disclosure is CL659 or a pharmaceutically acceptable salt thereof.

In some aspects, the EV, e.g., exosome, comprises a cyclic dinucleotide STING agonist and/or a non-cyclic dinucleotide STING agonist. In some aspects, when several cyclic dinucleotide STING agonist are present on an EV, e.g., exosome, disclosed herein, such STING agonists can be the same or they can be different. In some aspects, when several non-cyclic dinucleotide STING agonist are present, such STING agonists can be the same or they can be different. In some aspects, an EV, e.g., exosome, composition of the present disclosure can comprise two or more populations of EVs, e.g., exosomes, wherein each population of EVs, e.g., exosomes, comprises a different STING agonist or combination thereof.

In some aspects, one or more exogenous biologically active molecules, e.g., an adjuvant, is a TLR agonist. Non-limiting examples of TLR agonists include: TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), and combinations thereof. Non-limiting examples of TLR agonists can be found at WO2008115319A2, US20130202707A1, US20120219615A1, US20100029585A1, WO2009030996A1, WO2009088401A2, and WO2011044246A1, each of which are incorporated by reference in its entirety.

In some aspects, an EV (e.g., exosome) comprising a targeting moiety (e.g., those disclosed herein) and an adjuvant can comprise additional exogenous biologically active molecules (e.g., immune modulators).

### Immune Modulator

In some aspects, an EV, e.g., exosome, of the present disclosure have been modified or engineered to comprise one or more (e.g., two, three, four, five or more) immune modulators. In certain aspects, the one or more immune modulators are expressed in combination with other exogenous biologically active molecules disclosed herein (e.g., targeting moiety, therapeutic molecule, and/or adjuvant).

In some aspects, the present disclosure is directed to modified or engineered EVs comprising two or more exogenous biologically active molecules, wherein the two or more exogenous biologically active molecules are immune modulators, a first immune modulator and a second immune modulator (e.g., in addition to a targeting moiety disclosed herein). In some aspects, the first immune modulator is linked to a first Scaffold Y on the luminal surface of the EV, e.g., exosome, and the second immune modulator is linked to a second Scaffold Y on the luminal surface of the EV, e.g., exosome. In some aspects, the first immune modulator is linked to a Scaffold Y on the luminal surface of the EV, e.g., exosome, and the second immune modulator is in the lumen of the EV, e.g., exosome, not linked to any scaffold moiety. In some aspects, the first immune modulator is in the lumen of the EV, e.g., exosome, not linked to any scaffold moiety, and the second immune modulator is linked to a Scaffold Y on the luminal surface of the EV, e.g., exosome. In some aspects, the first immune modulator is linked to a Scaffold Y on the luminal surface of the EV, e.g., exosome, and the second immune modulator is linked to a Scaffold X on the exterior surface of the EV, e.g., exosome. In some aspects, the first immune modulator is in the lumen of the EV, e.g., exosome, not linked to any scaffold moiety, and the second immune modulator is linked to a Scaffold X on the exterior surface of the EV, e.g., exosome. In some aspects, the first immune modulator is linked to a Scaffold Y on the luminal surface of the EV, e.g., exosome, and the second immune modulator is linked to a Scaffold X on the luminal surface of the EV, e.g., exosome. In some aspects, the first immune modulator is in the lumen of the EV, e.g., exosome, not linked to any scaffold moiety, and the second immune modulator is linked to a Scaffold X on the luminal surface of the EV, e.g., exosome. In some aspects, the first immune modulator is linked to a Scaffold X on the luminal surface of the EV, e.g., exosome, and the second immune modulator is linked to the Scaffold X on the exterior surface of the EV, e.g., exosome. In some aspects, the first immune modulator is linked to a first Scaffold X on the exterior surface of the EV, e.g., exosome, and the second immune modulator is linked to a second Scaffold X on the exterior surface of the EV, e.g., exosome. In some aspects, the first immune modulator is linked to a Scaffold X on the exterior surface of the EV, e.g., exosome, and the second immune modulator is linked to a Scaffold Y on the luminal surface of the EV, e.g., exosome. In some aspects, the first immune modulator is linked to a Scaffold X on the exterior surface of the EV, e.g., exosome, and the second immune modulator is in the lumen of the EV, e.g., exosome, not linked to any scaffold moiety. In some aspects, the first immune modulator is linked to a Scaffold X on the exterior surface of the EV, e.g., exosome, and the second immune modulator is linked to the Scaffold X on the luminal surface of the EV, e.g., exosome. In some aspects, the first immune modulator is linked to a first Scaffold X on the luminal surface of the EV, e.g., exosome, and the second immune modulator is linked to a second Scaffold X on the luminal surface of the EV, e.g., exosome. In some aspects, the first immune modulator is linked to a Scaffold X on the luminal surface of the EV, e.g., exosome, and the second immune modulator is linked to a Scaffold Y on the luminal surface of the EV, e.g., exosome. In some aspects, the first immune modulator is linked to a Scaffold X on the luminal surface of the EV, e.g., exosome, and the second immune modulator is in the lumen of the EV, e.g., exosome, not linked to any scaffold moiety. In some aspects, the first immune modulator is linked to a first Scaffold X on the exterior surface of the EV, e.g., exosome, and the second immune modulator is linked to a second Scaffold X on the luminal surface of the EV, e.g., exosome. In some aspects, the first immune modulator is linked to a first Scaffold X on the luminal surface of the EV, e.g., exosome, and the second immune modulator is linked to a second Scaffold X on the exterior surface of the EV, e.g., exosome. In some aspects, the first immune modulator is in the lumen of the EV, e.g., exosome, not linked to any scaffold moiety, and the second immune modulator is in the lumen of the EV, e.g., exosome, not linked to any scaffold moiety.

In some aspects, an immune modulator that can be used with the EVs, e.g., exosomes, described herein has anti-tumor activity. In other aspects, an immune modulator useful for the present disclosure has tolerogenic activity. In some aspects, an immune modulator can regulate innate immune response. In certain aspects, an immune modulator regulates innate immune response by targeting natural killer cells. In some aspects, an immune modulator can regulate adaptive immune response. In some aspects, the immune modulator regulates adaptive immune response by targeting cytotoxic T cells. In further aspects, the immune modulator regulates adaptive immune response by targeting B cells. In certain aspects, an immune modulator disclosed herein can modulate the distribution of an exosome to a cytotoxic T cell or a B cell (i.e., bio-distribution modifying agent).

In some aspects, an immune modulator comprises an inhibitor for a negative checkpoint regulator or an inhibitor for a binding partner of a negative checkpoint regulator. In certain aspects, the negative checkpoint regulator comprises cytotoxic T-lymphocyte-associated protein 4 (CTLA-4), programmed cell death protein 1 (PD-1), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, or any combination thereof.

In some aspects, the immune modulator is an inhibitor of cytotoxic T-lymphocyte-associate protein 4 (CTLA-4). In certain aspects, the CTLA-4 inhibitor is a monoclonal antibody of CTLA-4 (“anti-CTLA-4 antibody”). In certain aspects, the inhibitor is a fragment of a monoclonal antibody of CTLA-4. In certain aspects, the antibody fragment is a scFv, (scFv)₂, Fab, Fab′, and F(ab′)₂, F(ab1)₂, Fv, dAb, or Fd of a monoclonal antibody of CTLA-4. In certain aspects, the inhibitor is a nanobody, a bispecific antibody, or a multispecific antibody against CTLA-4. In some aspects, the anti-CTLA-4 antibody is ipilimumab. In other aspects, the anti-CTLA-4 antibody is tremelimumab.

In some aspects, the immune modulator is an inhibitor of programmed cell death protein 1 (PD-1). In some aspects, the immune modulator is an inhibitor of programmed death-ligand 1 (PD-L1). In some aspects, the immune modulator is an inhibitor of programmed death-ligand 2 (PD-L2). In certain aspects, the inhibitor of PD-1, PD-L1, or PD-L2 is a monoclonal antibody of PD-1 (“anti-PD-1 antibody”), PD-L1 (“anti-PD-L1 antibody”), or PD-L2 (“anti-PD-L2 antibody”). In some aspects, the inhibitor is a fragment of an anti-PD-1 antibody, anti-PD-L1 antibody, or anti-PD-L2 antibody. In certain aspects, the antibody fragment is a scFv, (scFv)₂, Fab, Fab′, and F(ab′)₂, F(ab1)₂, Fv, dAb, or Fd of a monoclonal antibody of PD-1, PD-L1, or PD-L2. In certain aspects, the inhibitor is a nanobody, a bispecific antibody, or a multispecific antibody against PD-1, PD-L1, or PD-L2. In some aspects, the anti-PD-1 antibody is nivolumab. In some aspects, the anti-PD-1 antibody is pembrolizumab. In some aspects, the anti-PD-1 antibody is pidilizumab. In some aspects, the anti-PD-L1 antibody is atezolizumab. In other aspects, the anti-PD-L1 antibody is avelumab.

In some aspects, the immune modulator is an inhibitor of lymphocyte-activated gene 3 (LAG3). In certain aspects, the inhibitor of LAG3 is a monoclonal antibody of LAG3 (“anti-LAG3 antibody”). In some aspects, the inhibitor is a fragment of an anti-LAG3 antibody, e.g., scFv, (scFv)₂, Fab, Fab′, and F(ab′)₂, F(ab1)₂, Fv, dAb, or Fd. In certain aspects, the inhibitor is a nanobody, a bispecific antibody, or a multispecific antibody against LAG3.

In some aspects, the immune modulator is an inhibitor of T-cell immunoglobulin mucin-containing protein 3 (TIM-3). In some aspects, the immune modulator is an inhibitor of B and T lymphocyte attenuator (BTLA). In some aspects, the immune modulator is an inhibitor of T cell immunoreceptor with Ig and ITIM domains (TIGIT). In some aspects, the immune modulator is an inhibitor of V-domain Ig suppressor of T cell activation (VISTA). In some aspects, the immune modulator is an inhibitor of adenosine A2a receptor (A2aR). In some aspects, the immune modulator is an inhibitor of killer cell immunoglobulin like receptor (KIR). In some aspects, the immune modulator is an inhibitor of indoleamine 2,3-dioxygenase (IDO). In some aspects, the immune modulator is an inhibitor of CD20, CD39, or CD73.

In some aspects, the immune modulator comprises an activator for a positive co-stimulatory molecule or an activator for a binding partner of a positive co-stimulatory molecule. In certain aspects, the positive co-stimulatory molecule comprises a TNF receptor superfamily member (e.g., CD120a, CD120b, CD18, OX40, CD40, Fas receptor, M68, CD27, CD30, 4-1BB, TRAILR1, TRAILR2, TRAILR3, TRAILR4, RANK, OCIF, TWEAK receptor, TACI, BAFF receptor, ATAR, CD271, CD269, AITR, TROY, CD358, TRAMP, and XEDAR). In some aspects, the activator for a positive co-stimulatory molecule is a TNF superfamily member (e.g., TNFα, TNF-C, OX40L, CD40L, FasL, LIGHT, TL1A, CD27L, Siva, CD153, 4-1BB ligand, TRAIL, RANKL, TWEAK, APRIL, BAFF, CAMLG, NGF, BDNF, NT-3, NT-4, GITR ligand, and EDA-2).

In some aspects, the immune modulator is an activator of TNF Receptor Superfamily Member 4 (OX40). In certain aspects, the activator of OX40 is an agonistic anti-OX40 antibody. In further aspects, the activator of OX40 is a OX40 ligand (OX40L).

In some aspects, the immune modulator is an activator of CD27. In certain aspects, the activator of CD27 is an agonistic anti-CD27 antibody. In other aspects, the activator of CD27 is a CD27 ligand (CD27L).

In some aspects, the immune modulator is an activator of CD40. In certain aspects, the activator of CD40 is an agonistic anti-CD40 antibody. In some aspects, the activator of CD40 is a CD40 ligand (CD40L). In certain aspects, the CD40L is a monomeric CD40L. In other aspects, the CD40L is a trimeric CD40L.

In some aspects, the immune modulator is an activator of glucocorticoid-induced TNFR-related protein (GITR). In certain aspects, the activator of GITR is an agonistic anti-GITR antibody. In other aspects, the activator of GITR is a natural ligand of GITR.

In some aspects, the immune modulator is an activator of 4-1BB. In specific aspects, the activator of 4-1BB is an agonistic anti-4-1BB antibody. In certain aspects, the activator of 4-1BB is a natural ligand of 4-1BB.

In some aspects, the immune modulator is a Fas receptor (Fas). In such aspects, the Fas receptor is displayed on the surface of the EV, e.g., exosome. In some aspects, the immune modulator is Fas ligand (FasL). In certain aspects, the Fas ligand is displayed on the surface of the EV, e.g., exosome. In some aspects, the immune modulator is an anti-Fas antibody or an anti-FasL antibody.

In some aspects, the immune modulator is an activator of a CD28-superfamily co-stimulatory molecule. In certain aspects, the CD28-superfamily co-stimulatory molecule is ICOS or CD28. In certain aspects, the immune modulator is ICOSL, CD80, or CD86.

In some aspects, the immune modulator is an activator of inducible T cell co-stimulator (ICOS). In certain aspects, the activator of ICOS is an agonistic anti-ICOS antibody. In other aspects, the activator of ICOS is a ICOS ligand (ICOSL).

In some aspects, the immune modulator is an activator of CD28. In some aspects, the activator of CD28 is an agonistic anti-CD28 antibody. In other aspects, the activator of CD28 is a natural ligand of CD28. In certain aspects, the ligand of CD28 is CD80.

In some aspects, the immune modulator comprises a cytokine or a binding partner of a cytokine. In certain aspects, the cytokine comprises IL-2, IL-4, IL-7, IL-10, IL-12, IL-15, IL-21, or IFN-γ. In some aspects, the immune modulator comprises FLT-3 (CD135).

In some aspects, an EVs, e.g., exosomes, described herein comprises a first scaffold moiety. In certain aspects, a first exogenous biologically active molecule (e.g., targeting moiety, therapeutic molecule, adjuvant, or immune modulator) is linked to the first scaffold moiety. In other aspects, a second exogenous biologically active molecule (e.g., targeting moiety, therapeutic molecule, adjuvant, or immune modulator) is linked to the first scaffold moiety. In further aspects, both the first and second exogenous biologically active molecules are linked to the first scaffold moiety. In some aspects, an EVs, e.g., exosomes, further comprises a second scaffold moiety. In certain aspects, the first exogenous biologically active molecule is linked to the first scaffold moiety, and the second exogenous biologically active molecule is linked to the second scaffold moiety. In some aspects, the first scaffold moiety and the second scaffold moiety are the same (e.g., both Scaffold X or both Scaffold Y). In other aspects, the first scaffold moiety and the second scaffold moiety are different (e.g., first scaffold moiety is Scaffold X and the second scaffold moiety is Scaffold Y; or first scaffold moiety is Scaffold Y and the second scaffold moiety is Scaffold X).

Non-limiting examples of Scaffold X include: prostaglandin F2 receptor negative regulator (PTGFRN); basigin (BSG); immunoglobulin superfamily member 2 (IGSF2); immunoglobulin superfamily member 3 (IGSF3); immunoglobulin superfamily member 8 (IGSF8); integrin beta-1 (ITGB1); integrin alpha-4 (ITGA4); 4F2 cell-surface antigen heavy chain (SLC3A2); and a class of ATP transporter proteins (ATP1A1, ATP1A2, ATP1A3, ATP1A4, ATP1B3, ATP2B1, ATP2B2, ATP2B3, ATP2B). In certain aspects, Scaffold X is a whole protein. In other aspects, Scaffold X is a protein fragment (e.g., functional fragment).

In other aspects, the scaffold moiety useful for the present disclose, a first scaffold moiety, a second scaffold moiety, and/or a third scaffold moiety, includes a conventional exosome protein, including, but not limiting, tetraspanin molecules (e.g., CD63, CD81, CD9 and others), lysosome-associated membrane protein 2 (LAMP2 and LAMP2B), platelet-derived growth factor receptor (PDGFR), GPI anchor proteins, lactadherin and fragments thereof, peptides that have affinity to any of these proteins or fragments thereof, or any combination thereof.

Non-limiting examples of Scaffold Y include: the myristoylated alanine rich Protein Kinase C substrate (MARCKS) protein; myristoylated alanine rich Protein Kinase C substrate like 1 (MARCKSL1) protein; and brain acid soluble protein 1 (BASP1) protein. In some aspects, Scaffold Y is a whole protein. In certain aspects, Scaffold Y is a protein fragment (e.g., functional fragment).

In some aspects, an EV, e.g., exosome, of the present disclosure comprises two or more exogenous biologically active molecules, e.g., (i) one or more therapeutic molecules (e.g., antigens) and (ii) one or more targeting moieties, wherein the one or more therapeutic molecules are linked to a Scaffold Y on the luminal surface of the EV, e.g., exosome, and the one or more targeting moieties are linked to a Scaffold X on the exterior surface of the EV, e.g., exosome. In some aspects, an EV, e.g., exosome, of the present disclosure comprises two or more exogenous biologically active molecules, e.g., (i) one or more therapeutic molecules (e.g., antigens) and (ii) one or more targeting moieties, wherein the one or more therapeutic molecules are in the lumen of the EV, e.g., exosome, not linked to any scaffold moiety, and the one or more targeting moieties are linked to a Scaffold X on the exterior surface of the EV, e.g., exosome. In some aspects, an EV, e.g., exosome, of the present disclosure comprises two or more exogenous biologically active molecules, e.g., (i) one or more therapeutic molecules (e.g., antigens) and (ii) one or more targeting moieties, wherein the one or more therapeutic molecules are linked to a Scaffold X on the luminal surface of the EV, e.g., exosome, and the one or more targeting moieties are linked to the Scaffold X on the exterior surface of the EV, e.g., exosome. In some aspects, an EV, e.g., exosome, of the present disclosure comprises two or more exogenous biologically active molecules, e.g., (i) one or more therapeutic molecules (e.g., antigens) and (ii) one or more targeting moieties, wherein the one or more therapeutic molecules are linked to a first Scaffold X on the exterior surface of the EV, e.g., exosome, and the one or more targeting moieties are linked to a second Scaffold X on the exterior surface of the EV, e.g., exosome. In some aspects, an EV, e.g., exosome, of the present disclosure comprises two or more exogenous biologically active molecules, e.g., (i) one or more therapeutic molecules (e.g., antigens) and (ii) one or more targeting moieties, wherein the one or more therapeutic molecules are linked to a first Scaffold X on the luminal surface of the EV, e.g., exosome, and the one or more targeting moieties are linked to a second Scaffold X on the exterior surface of the EV, e.g., exosome.

In some aspects, the one or more exogenous biologically active molecule disclosed herein (e.g., targeting moiety, therapeutic molecule, immune modulator, or adjuvant) can be modified to increase encapsulation in an EV, e.g., exosome. This modification can include the addition of a lipid binding tag by treating the agonist with a chemical or enzyme, or by physically or chemically altering the polarity or charge of the exogenous biologically active molecule (e.g., adjuvant and/or antigen). The exogenous biologically active molecule can be modified by a single treatment, or by a combination of treatments, e.g., adding a lipid binding tag only, or adding a lipid binding tag and altering the polarity. The previous example is meant to be a non-limiting illustrative instance. It is contemplated that any combination of modifications can be practiced. The modification can increase encapsulation of the exogenous biologically active molecule in the EV, e.g., exosome by between 2-fold and 10,000 fold, between 10-fold and 1,000 fold, or between 100-fold and 500-fold compared to encapsulation of an unmodified exogenous biologically active molecule. The modification can increase encapsulation of the exogenous biologically active molecule in the EV, e.g., exosome by at least 2-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, or 10,000-fold compared to encapsulation of an unmodified exogenous biologically active molecule.

Additional non-limiting examples of specific aspects include EVs, e.g., exosomes, comprising (i) one or more targeting moieties, (ii) one or more therapeutic molecules (e.g., tumor antigens), and (iii) one or more adjuvants (e.g., a STING agonist or a TLR agonist) and/or immune modulators, wherein:
(a) the one or more targeting moieties are linked to a first Scaffold X on the exterior surface of the EV, e.g., exosome, the one or more therapeutic molecules are linked to a second Scaffold X on the exterior surface of the EV, e.g., exosome, and the one or more adjuvants and/or immune modulators are (a1) in the lumen of the EV, e.g., exosome, not linked to any scaffold moiety, or (a2) linked to a third scaffold moiety, e.g., a Scaffold X on the exterior surface of the exosome or on the luminal surface of the exosome or a Scaffold Y on the luminal surface of the EV, e.g., exosome;
(b) the one or more targeting moieties are linked to a Scaffold X on the exterior surface of the EV, e.g., exosome, the one or more therapeutic molecules are linked to a Scaffold Y on the luminal surface of the EV, e.g., exosome, and the one or more adjuvants and/or immune modulators are (bi) in the lumen of the EV, e.g., exosome, not linked to any scaffold moiety, or (b2) linked to a third scaffold moiety, e.g., a Scaffold X on the exterior surface of the exosome or on the luminal surface of the exosome or a Scaffold Y on the luminal surface of the EV, e.g., exosome;
(c) the one or more targeting moieties are linked to a Scaffold X on the exterior surface of the EV, e.g., exosome, the one or more therapeutic molecules are in the lumen of the EV, e.g., exosome, not linked to any scaffold moiety, and the one or more adjuvants and/or immune modulators are (c1) in the lumen of the EV, e.g., exosome, not linked to any scaffold moiety, or (c2) linked to a scaffold moiety, e.g., a Scaffold X on the exterior surface of the exosome or on the luminal surface of the exosome or a Scaffold Y on the luminal surface of the EV, e.g., exosome;
(d) the one or more targeting moieties are linked to a Scaffold X on the exterior surface of the EV, e.g., exosome, the one or more therapeutic molecules are linked to the Scaffold X on the luminal surface of the EV, e.g., exosome, and the one or more adjuvants and/or immune modulators are (d1) in the lumen of the EV, e.g., exosome, not linked to any scaffold moiety, or (d2) linked to a scaffold moiety, e.g., a Scaffold X on the exterior surface of the exosome or on the luminal surface of the exosome or a Scaffold Y on the luminal surface of the EV, e.g., exosome; or
(e) the one or more targeting moieties are linked to a first Scaffold X on the exterior surface of the EV, e.g., exosome, the one or more therapeutic molecules are linked to a second Scaffold X on the luminal surface of the EV, e.g., exosome, and the one or more adjuvants and/or immune modulators are (e1) in the lumen of the EV, e.g., exosome, not linked to any scaffold moiety, or (e2) linked to a third scaffold moiety, e.g., a Scaffold X on the surface of the exosome or in the lumen of the exosome or a Scaffold Y on the luminal surface of the EV, e.g., exosome.

In some aspects, the immune modulator comprises a protein that supports intracellular interactions required for germinal center responses. In certain aspects, such a protein comprises a signaling lymphocyte activation molecule (SLAM) family member or a SLAM-associated protein (SAP). In some aspects, a SLAM family members comprises SLAM, CD48, CD229 (Ly9), Ly108, 2B4, CD84, NTB-A, CRACC, BLAME, CD2F-10, or combinations thereof.

In some aspects, the immune modulator comprises a T-cell receptor (TCR) or a derivative thereof. In certain aspects, the immune modulator is a TCR α-chain or a derivative thereof. In other aspects, the immune modulator is a TCR β-chain or a derivative thereof. In further aspects, the immune modulator is a co-receptor of the T-cell or a derivative thereof.

In some aspects, the immune modulator comprises a chimeric antigen receptor (CAR) or a derivative thereof. In certain aspects, the CAR binds to one or more of the therapeutic molecules disclosed herein (e.g., tumor antigen, e.g., alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen, MART-1 gp100, and TNF-related apoptosis-inducing ligand).

In certain aspects, the immune modulator is an activator of CD28. In certain aspects, the activator is a fragment of a monoclonal antibody of CD28. In certain aspects, the antibody fragment is a scFv, (scFv)₂, Fab, Fab′, and F(ab′)₂, F(ab1)₂, Fv, dAb, or Fd of a monoclonal antibody of CD28. In certain aspects, the activator is a nanobody, a bispecific antibody, or a multispecific antibody against CD28.

In some aspects, the immune modulator comprises a NF-κB inhibitor. Non-limiting examples of NF-κB inhibitors that can be used with the present disclosure includes: IKK complex inhibitors (e.g., TPCA-1, NF-κB Activation Inhibitor VI (BOT-64), BMS 345541, Amlexanox, SC-514 (GK 01140), IMD 0354, IKK-16), IκB degradation inhibitor (e.g., BAY 11-7082, MG-115, MG-132, Lactacystin, Epoxomicin, Parthenolide, Carfilzomib, MLN-4924 (Pevonedistat)), NF-κB nuclear translocation inhibitor (e.g., JSH-23, Rolipram), p65 acetylation inhibitor (e.g., Gallic acid, Anacardic acid), NF-κB-DNA binding inhibitor (e.g., GYY 4137, p-XSC, CV 3988, Prostaglandin E2 (PGE2)), NF-κB transactivation inhibitor (e.g., LY 294002, Wortmannin, Mesalamine), or combinations thereof. See also Gupta, S.C., et al., Biochim Biophys Acta 1799:775-787 (2010), which is herein incorporated by reference in its entirety. In further aspects, an immune modulator comprises a COX-2 inhibitor, mTOR inhibitor (e.g., rapamycin and derivatives), prostaglandins, nonsteroidal anti-inflammatory agents (NSAIDS), antileukotriene, or combinations thereof.

In some aspects, the immune modulator is an agonist. In certain aspects, the agonist is an endogenous agonist, such as a hormone, or a neurotransmitter. In other aspects, the agonist is an exogenous agonist, such as a drug. In some aspects, the agonist is a physical agonist, which can create an agonist response without binding to the receptor. In some aspects, the agonist is a superagonist, which can produce a greater maximal response than the endogenous agonist. In certain aspects, the agonist is a full agonist with full efficacy at the receptor. In other aspects, the agonist is a partial agonist having only partial efficacy at the receptor relative to a full agonist. In some aspects, the agonist is an inverse agonist that can inhibit the constitutive activity of the receptor. In some aspects, the agonist is a co-agonist that works with other co-agonists to produce an effect on the receptor. In certain aspects, the agonist is an irreversible agonist that binds permanently to a receptor through formation of covalent bond. In certain aspects, the agonist is selective agonist for a specific type of receptor

In some aspects, the immune modulator is an antagonist. In specific aspects, the antagonist is a competitive antagonist, which reversibly binds to the receptor at the same binding site as the endogenous ligand or agonist without activating the receptor. Competitive antagonist can affect the amount of agonist necessary to achieve a maximal response. In other aspects, the antagonist is a non-competitive antagonist, which binds to an active site of the receptor or an allosteric site of the receptor. Non-competitive antagonist can reduce the magnitude of the maximum response that can be attained by any amount of agonist. In further aspects, the antagonist is an uncompetitive antagonist, which requires receptor activation by an agonist before its binding to a separate allosteric binding site.

In some aspects, the immune modulator comprises an antibody or an antigen-binding fragment. The immune modulator can be a full length protein or a fragment thereof. The antibody or antigen-binding fragment can be derived from natural sources, or partly or wholly synthetically produced. In some aspects, the antibody is a monoclonal antibody. In some of these aspects, the monoclonal antibody is an IgG antibody. In certain aspects, the monoclonal antibody is an IgG1, IgG2, IgG3, or IgG4. In some other aspects, the antibody is a polyclonal antibody. In certain aspects, the antigen-binding fragment is selected from Fab, Fab′, and F(ab′)₂, F(ab1)₂, Fv, dAb, and Fd fragments. In certain aspects, the antigen-binding fragment is an scFv or (scFv)₂ fragment. In certain other aspects, the antibody or antigen-binding fragment is a NANOBODY® (single-domain antibody). In some aspects, the antibody or antigen-binding fragment is a bispecific or multispecific antibody.

In various aspects, the antibody or antigen-binding fragment is fully human. In some aspects, the antibody or antigen-binding fragment is humanized. In some aspects, the antibody or antigen-binding fragment is chimeric. In some of these aspects, the chimeric antibody has non-human V region domains and human C region domains. In some aspects, the antibody or antigen-binding fragment is non-human, such as murine or veterinary.

In certain aspects, the immune modulator is a polynucleotide. In some of these aspects, the polynucleotide includes, but is not limited to, an mRNA, a miRNA, an siRNA, an antisense RNA, an shRNA, a lncRNA, and a dsDNA. In some aspects, the polynucleotide is an RNA (e.g., an mRNA, a miRNA, an siRNA, an antisense RNA, an shRNA, or an lncRNA). In some of these aspects, when the polynucleotide is an mRNA, it can be translated into a desired polypeptide. In some aspects, the polynucleotide is a microRNA (miRNA) or pre-miRNA molecule. In some of these aspects, the miRNA is delivered to the cytoplasm of the target cell, such that the miRNA molecule can silence a native mRNA in the target cell. In some aspects, the polynucleotide is a small interfering RNA (siRNA) or a short hairpin RNA (shRNA) capable of interfering with the expression of an oncogene or other dysregulating polypeptides. In some of these aspects, the siRNA is delivered to the cytoplasm of the target cell, such that the siRNA molecule can silence a native mRNA in the target cell. In some aspects, the polynucleotide is an antisense RNA that is complementary to an mRNA. In some aspects, the polynucleotide is a long non-coding RNA (lncRNA) capable of regulating gene expression and modulating diseases. In some aspects, the polynucleotide is a DNA that can be transcribed into an RNA. In some of these aspects, the transcribed RNA can be translated into a desired polypeptide.

In some aspects, the immune modulator is a protein, a peptide, a glycolipid, or a glycoprotein.

In various aspects, the composition comprises two or more above mentioned immune modulators, including mixtures, fusions, combinations and conjugates, of atoms, molecules, etc. In some aspects, the composition comprises one, two, three, four, five, six, seven, eight, nine, ten, eleven, or twelve different immune modulators associated with the membrane or enclosed within the enclosed volume of said extracellular vesicle. In certain aspects, the composition comprises a nucleic acid combined with a polypeptide. In certain aspects, the composition comprises two or more polypeptides conjugated to each other. In certain aspects, the composition comprises a protein conjugated to an exogenous biologically active molecule. In some of these aspects, the exogenous biologically active molecule is a prodrug.

In some aspects, an EV (e.g., exosome) disclosed herein comprises a targeting moiety and a STING agonist. In some aspects, an EV (e.g., exosome) disclosed herein comprises a targeting moiety and a TLR agonist (e.g., TLR3 agonist). In some aspects, an EV (e.g., exosome) disclosed herein comprises a targeting moiety and IFN-α or IFN-γ. In some aspects, the targeting moiety specifically binds to aClec9a protein (or a variant thereof). In each of these aspects, a targeting moiety can comprise an antigen, an immunosuppressive agent, or both.

### Scaffold X-Engineered EVs, e.g., Exosomes,

In some aspects, EVs, e.g., exosomes, of the present disclosure comprise a membrane modified in its composition. For example, their membrane compositions can be modified by changing the protein, lipid, or glycan content of the membrane.

In some aspects, the surface-engineered EVs, e.g., exosomes, are generated by chemical and/or physical methods, such as PEG-induced fusion and/or ultrasonic fusion. In other aspects, the surface-engineered EVs, e.g., exosomes, are generated by genetic engineering. EVs, e.g., exosomes, produced from a genetically-modified producer cell or a progeny of the genetically-modified cell can contain modified membrane compositions. In some aspects, surface-engineered EVs, e.g., exosomes, have scaffold moiety (e.g., exosome protein, e.g., Scaffold X) at a higher or lower density (e.g., higher number) or include a variant or a fragment of the scaffold moiety. In certain aspects, surface-engineered EVs, e.g., exosomes, can comprise multiple (e.g., two or more) scaffold moieties on their exterior surface. In some aspects, each of the multiple scaffold moieties are the same. In other aspects, one or more of the multiple scaffold moieties are different.

For example, surface (e.g., Scaffold X)-engineered EVs, can be produced from a cell (e.g., HEK293 cells) transformed with an exogenous sequence encoding a scaffold moiety (e.g., exosome proteins, e.g., Scaffold X) or a variant or a fragment thereof. EVs including scaffold moiety expressed from the exogenous sequence can include modified membrane compositions.

Various modifications or fragments of the scaffold moiety can be used for the aspects of the present disclosure. For example, one or more scaffold moieties modified to have enhanced affinity to a binding agent can be used for generating surface-engineered EV that can be purified using the binding agent. Scaffold moieties modified to be more effectively targeted to EVs and/or membranes can be used. Scaffold moieties modified to comprise a minimal fragment required for specific and effective targeting to exosome membranes can be also used.

Scaffold moieties can be engineered to be expressed as a fusion molecule, e.g., fusion molecule of Scaffold X to one or more exogenous biologically active molecules (e.g., those disclosed herein, e.g., a therapeutic molecule (e.g., an antigen), an adjuvant, and/or an immune modulator). For example, the fusion molecule can comprise a scaffold moiety disclosed herein (e.g., Scaffold X, e.g., PTGFRN, BSG, IGSF2, IGSF3, IGSF8, ITGB1, ITGA4, SLC3A2, ATP transporter, or a fragment or a variant thereof) linked to a therapeutic molecule (e.g., antigen), an adjuvant, and/or an immune modulator. In case of the fusion molecule, the therapeutic molecule, adjuvant, and/or immune modulator can be a natural peptide, a recombinant peptide, a synthetic peptide, or any combination thereof.

In some aspects, the surface (e.g., Scaffold X)-engineered EVs described herein demonstrate superior characteristics compared to EVs known in the art. For example, surface (e.g., Scaffold X)-engineered contain modified proteins more highly enriched on their surface than naturally occurring EVs or the EVs produced using conventional exosome proteins. In some aspects, surface (e.g., Scaffold X)-engineered EVs described herein can express greater number (e.g., 2, 3, 4, 5 or more) of exogenous biologically active molecules, such that multiple EVs are not required. Moreover, the surface (e.g., Scaffold X)-engineered EVs of the present invention can have greater, more specific, or more controlled biological activity compared to naturally occurring EVs or the EVs produced using conventional exosome proteins.

In some aspects the Scaffold X comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN protein (Uniprot Accession No. Q9P2B2) is shown at Table 1 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least five, at least 10, at least 15, at least 20, at least 25, at least 30, at least 40, at least 50, at least 70, at least 80, at least 90, at least 100, at least 110, at least 120, at least 130, at least 140, at least 150 amino acids at the N terminus of the transmembrane domain, (ii) at least five, at least 10, at least 15, at least 20, or at least 25 amino acids at the C terminus of the transmembrane domain, or both (i) and (ii).

In some aspects, the fragments of PTGFRN polypeptide lack one or more functional or structural domains, such as IgV.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 33. In other aspects, the Scaffold X comprises the amino acid sequence of SEQ ID NO: 33, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 33 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 33.

In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 2, 3, 4, 5, 6, or 7. In other aspects, the Scaffold X comprises the amino acid sequence of SEQ ID NO: 2, 3, 4, 5, 6, or 7, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 2, 3, 4, 5, 6, or 7 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 2, 3, 4, 5, 6, or 7.

TABLE 1
Exemplary Scaffold X Protein Sequences

| Protein | Sequence |
|---|---|
| The PTGFRN | MGRLASRPLLLALLSLALCRGRVVRVPTATLVRVVGTELVIPCNVSDYDGPSEQNFDWSF |
| Protein | SSLGSSFVELASTWEVGFPAQLYQERLQRGEILLRRTANDAVELHIKNVQPSDQGHYKCS |
| (SEQ ID NO: 1) | TPSTDATVQGNYEDTVQVKVLADSLHVGPSARPPPSLSLREGEPFELRCTAASASPLHTH |
| | LALLWEVHRGPARRSVLALTHEGRFHPGLGYEQRYHSGDVRLDTVGSDAYRLSVSRALSA |
| | DQGSYRCIVSEWIAEQGNWQEIQEKAVEVATVVIQPSVLRAAVPKNVSVAEGKELDLTCN |
| | ITTDRADDVRPEVTWSFSRMPDSTLPGSRVLARLDRDSLVHSSPHVALSHVDARSYHLLV |
| | RDVSKENSGYYYCHVSLWAPGHNRSWHKVAEAVSSPAGVGVTWLEPDYQVYLNASKVPGF |
| | ADDPTELACRVVDTKSGEANVRFTVSWYYRMNRRSDNVVTSELLAVMDGDWTLKYGERSK |
| | QRAQDGDFIFSKEHTDTFNFRIQRTTEEDRGNYYCVVSAWTKQRNNSWVKSKDVFSKPVN |
| | IFWALEDSVLVVKARQPKPFFAAGNTFEMTCKVSSKNIKSPRYSVLIMAEKPVGDLSSPN |
| | ETKYIISLDQDSVVKLENWTDASRVDGVVLEKVQEDEFRYRMYQTQVSDAGLYRCMVTAW |
| | SPVRGSLWREAATSLSNPIEIDFQTSGPIFNASVHSDTPSVIRGDLIKLFCIITVEGAAL |
| | DPDDMAFDVSWFAVHSFGLDKAPVLLSSLDRKGIVTTSRRDWKSDLSLERVSVLEFLLQV |
| | HGSEDQDFGNYYCSVTPWVKSPTGSWQKEAEIHSKPVFITVKMDVLNAFKYPLLIGVGLS |
| | TVIGLLSCLIGYCSSHWCCKKEVQETRRERRRLMSMEMD |
| The PTGFRN | GPIFNASVHSDTPSVIRGDLIKLFCIITVEGAALDPDDMAFDVSWFAVHSFGLDKAPVLL |
| protein | SSLDRKGIVTTSRRDWKSDLSLERVSVLEFLLQVHGSEDQDFGNYYCSVTPWVKSPTGSW |
| Fragment | QKEAEIHSKPVFITVKMDVLNAFKYPLLIGVGLSTVIGLLSCLIGYCSSHWCCKKEVQET |
| (SEQ ID NO: 33) | RRERRRLMSMEM |
| | 687-878 of SEQ ID NO: 1 |
| The BSG | MAAALFVLLG FALLGTHGAS GAAGFVQAPL SQQRWVGGSV ELHCEAVGSP |
| protein | VPEIQWWFEG QGPNDTCSQL WDGARLDRVH IHATYHQHAA STISIDTLVE |
| (SEQ ID NO: 9) | EDTGTYECRA SNDPDRNHLT RAPRVKWVRA QAVVLVLEPG TVFTTVEDLG |
| | SKILLTCSLN DSATEVTGHR WLKGGVVLKE DALPGQKTEF KVDSDDQWGE |
| | YSCVFLPEPM GTANIQLHGP PRVKAVKSSE HINEGETAML VCKSESVPPV |
| | TDWAWYKITD SEDKALMNGS ESRFFVSSSQ GRSELHIENL NMEADPGQYR |
| | CNGTSSKGSD QAIITLRVRS HLAALWPFLG IVAEVLVLVT IIFIYEKRRK |
| | PEDVLDDDDA GSAPLKSSGQ HQNDKGKNVR QRNSS |
| The IGSF8 | MGALRPTLLP PSLPLLLLLM LGMGCWAREV LVPEGPLYRV AGTAVSISCN |
| protein | VTGYEGPAQQ NFEWFLYRPE APDTALGIVS TKDTQFSYAV FKSRVVAGEV |
| (SEQ ID NO: 14) | QVQRLQGDAV VLKIARLQAQ DAGIYECHTP STDTRYLGSY SGKVELRVLP |
| | DVLQVSAAPP GPRGRQAPTS PPRMTVHEGQ ELALGCLART STQKHTHLAV |
| | SFGRSVPEAP VGRSTLQEVV GIRSDLAVEA GAPYAERLAA GELRLGKEGT |
| | DRYRMVVGGA QAGDAGTYHC TAAEWIQDPD GSWAQIAEKR AVLAHVDVQT |
| | LSSQLAVTVG PGERRIGPGE PLELLCNVSG ALPPAGRHAA YSVGWEMAPA |
| | GAPGPGRLVA QLDTEGVGSL GPGYEGRHIA MEKVASRTYR LRLEAARPGD |
| | AGTYRCLAKA YVRGSGTRLR EAASARSRPL PVHVREEGVV LEAVAWLAGG |
| | TVYRGETASL LCNISVRGGP PGLRLAASWW VERPEDGELS SVPAQLVGGV |
| | GQDGVAELGV RPGGGPVSVE LVGPRSHRLR LHSLGPEDEG VYHCAPSAWV |
| | QHADYSWYQA GSARSGPVTV YPYMHALDTL FVPLLVGTGV ALVTGATVLG |
| | TITCCFMKRL RKR |
| The ITGB1 | MNLQPIFWIG LISSVCCVFA QTDENRCLKA NAKSCGECIQ AGPNCGWCTN |
| protein | STFLQEGMPT SARCDDLEAL KKKGCPPDDI ENPRGSKDIK KNKNVTNRSK |
| (SEQ ID NO: 21) | GTAEKLKPED ITQIQPQQLV LRLRSGEPQT FTLKFKRAED YPIDLYYLMD |
| | LSYSMKDDLE NVKSLGTDLM NEMRRITSDF RIGFGSFVEK TVMPYISTTP |
| | AKLRNPCTSE QNCTSPFSYK NVLSLINKGE VFNELVGKQR ISGNLDSPEG |
| | GFDAIMQVAV CGSLIGWRNV TRLLVFSTDA GFHFAGDGKL GGIVLPNDGQ |
| | CHLENNMYTM SHYYDYPSIA HLVQKLSENN IQTIFAVTEE FQPVYKELKN |
| | LIPKSAVGTL SANSSNVIQL IIDAYNSLSS EVILENGKLS EGVTISYKSY |
| | CKNGVNGTGE NGRKCSNISI GDEVQFEISI TSNKCPKKDS DSFKIRPLGF |
| | TEEVEVILQY ICECECQSEG IPESPKCHEG NGTFECGACR CNEGRVGRHC |
| | ECSTDEVNSE DMDAYCRKEN SSEICSNNGE CVCGQCVCRK RDNTNEIYSG |
| | ASNGQICNGR GICECGVCKC TDPKFQGQTC EMCQTCLGVC AEHKECVQCR |
| | AFNKGEKKDT CTQECSYFNI TKVESRDKLP QPVQPDPVSH CKEKDVDDCW |
| | FYFTYSVNGN NEVMVHVVEN PECPTGPDII PIVAGVVAGI VLIGLALLLI |
| | WKLLMIIHDR REFAKFEKEK MNAKWDTGEN PIYKSAVTTV VNPKYEGK |
| The ITGA4 | MAWEARREPG PRRAAVRETV MLLLCLGVPT GRPYNVDTES ALLYQGPHNT |
| protein | LFGYSVVLHS HGANRWLLVG APTANWLANA SVINPGAIYR CRIGKNPGQT |
| (SEQ ID NO: 22) | CEQLQLGSPN GEPCGKTCLE ERDNQWLGVT LSRQPGENGS IVTCGHRWKN |
| | IFYIKNENKL PTGGCYGVPP DLRTELSKRI APCYQDYVKK FGENFASCQA |
| | GISSFYTKDL IVMGAPGSSY WTGSLFVYNI TINKYKAFLD KQNQVKFGSY |
| | LGYSVGAGHF RSQHTTEVVG GAPQHEQIGK AYIFSIDEKE LNILHEMKGK |
| | KLGSYFGASV CAVDLNADGF SDLLVGAPMQ STIREEGRVF VYINSGSGAV |
| | MNAMETNLVG SDKYAARFGE SIVNLGDIDN DGFEDVAIGA PQEDDLQGAI |
| | YIYNGRADGI SSTFSQRIEG LQISKSLSMF GQSISGQIDA DNNGYVDVAV |
| | GAFRSDSAVL LRTRPVVIVD ASLSHPESVN RTKFDCVENG WPSVCIDLTL |
| | CFSYKGKEVP GYIVLFYNMS LDVNRKAESP PRFYFSSNGT SDVITGSIQV |
| | SSREANCRTH QAFMRKDVRD ILTPIQIEAA YHLGPHVISK RSTEEFPPLQ |
| | PILQQKKEKD IMKKTINFAR FCAHENCSAD LQVSAKIGFL KPHENKTYLA |
| | VGSMKTLMLN VSLFNAGDDA YETTLHVKLP VGLYFIKILE LEEKQINCEV |
| | TDNSGVVQLD CSIGYIYVDH LSRIDISFLL DVSSLSRAEE DLSITVHATC |
| | ENEEEMDNLK HSRVTVAIPL KYEVKLTVHG FVNPTSFVYG SNDENEPETC |
| | MVEKMNLTFH VINTGNSMAP NVSVEIMVPN SFSPQTDKLF NILDVQTTTG |
| | ECHFENYQRV CALEQQKSAM QTLKGIVRFL SKTDKRLLYC IKADPHCLNF |
| | LCNFGKMESG KEASVHIQLE GRPSILEMDE TSALKFEIRA TGFPEPNPRV |
| | IELNKDENVA HVLLEGLHHQ RPKRYFTIVI ISSSLLLGLI VLLLISYVMW |
| | KAGFFKRQYK SILQEENRRD SWSYINSKSN DD |
| The SLC3A2 | MELQPPEASI AVVSIPRQLP GSHSEAGVQG LSAGDDSELG SHCVAQTGLE |
| Protein, where | LLASGDPLPS ASQNAEMIET GSDCVTQAGL QLLASSDPPA LASKNAEVTG |
| the first Met is | TMSQDTEVDM KEVELNELEP EKQPMNAASG AAMSLAGAEK NGLVKIKVAE |
| processed. | DEAEAAAAAK FTGLSKEELL KVAGSPGWVR TRWALLLLFW LGWLGMLAGA |
| (SEQ ID NO: 23) | VVIIVRAPRC RELPAQKWWH TGALYRIGDL QAFQGHGAGN LAGLKGRLDY |
| | LSSLKVKGLV LGPIHKNQKD DVAQTDLLQI DPNFGSKEDF DSLLQSAKKK |
| | SIRVILDLTP NYRGENSWFS TQVDTVATKV KDALEFWLQA GVDGFQVRDI |
| | ENLKDASSFL AEWQNITKGF SEDRLLIAGT NSSDLQQILS LLESNKDLLL |
| | TSSYLSDSGS TGEHTKSLVT QYLNATGNRW CSWSLSQARL LTSFLPAQLL |
| | RLYQLMLFTL PGTPVFSYGD EIGLDAAALP GQPMEAPVML WDESSFPDIP |
| | GAVSANMTVK GQSEDPGSLL SLFRRLSDQR SKERSLLHGD FHAFSAGPGL |
| | FSYIRHWDQN ERFLVVLNFG DVGLSAGLQA SDLPASASLP AKADLLLSTQ |
| | PGREEGSPLE LERLKLEPHE GLLLRFPYAA |

In some aspects, a Scaffold X comprises Basigin (the BSG protein), represented by SEQ ID NO: 9. The BSG protein is also known as 5F7, Collagenase stimulatory factor, Extracellular matrix metalloproteinase inducer (EMMPRIN), Leukocyte activation antigen M6, OK blood group antigen, Tumor cell-derived collagenase stimulatory factor (TCSF), or CD147. The Uniprot number for the human BSG protein is P35613. The signal peptide of the BSG protein is amino acid 1 to 21 of SEQ ID NO: 9. Amino acids 138-323 of SEQ ID NO: 9 is the extracellular domain, amino acids 324 to 344 is the transmembrane domain, and amino acids 345 to 385 of SEQ ID NO: 9 is the cytoplasmic domain.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 22 to 385 of SEQ ID NO: 9. In some aspects, the fragments of BSG polypeptide lack one or more functional or structural domains, such as IgV, e.g., amino acids 221 to 315 of SEQ ID NO: 9. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 10, 11, or 12. In other aspects, the Scaffold X comprises the amino acid sequence of SEQ ID NO: 10, 11, or 12, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 10, 11, or 12 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 10, 11, or 12.

In some aspects, a Scaffold X comprises Immunoglobulin superfamily member 8 (IgSF8 or the IGSF8 protein), which is also known as CD81 partner 3, Glu-Trp-Ile EWI motif-containing protein 2 (EWI-2), Keratinocytes-associated transmembrane protein 4 (KCT-4), LIR-D1, Prostaglandin regulatory-like protein (PGRL) or CD316. The full length human IGSF8 protein is accession no. Q969P0 in Uniprot and is shown as SEQ ID NO: 14 herein. The human IGSF8 protein has a signal peptide (amino acids 1 to 27 of SEQ ID NO: 14), an extracellular domain (amino acids 28 to 579 of SEQ ID NO: 14), a transmembrane domain (amino acids 580 to 600 of SEQ ID NO: 14), and a cytoplasmic domain (amino acids 601 to 613 of SEQ ID NO: 14).

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 28 to 613 of SEQ ID NO: 14. In some aspects, the IGSF8 protein lack one or more functional or structural domains, such as IgV. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 15, 16, 17, or 18. In other aspects, the Scaffold X comprises the amino acid sequence of SEQ ID NO: 15, 16, 17, or 18, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID 15, 16, 17, or 18 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 15, 16, 17, or 18.

In some aspects, a Scaffold X for the present disclosure comprises Immunoglobulin superfamily member 3 (IgSF3 or the IGSF3 protein), which is also known as Glu-Trp-Ile EWI motif-containing protein 3 (EWI-3), and is shown as the amino acid sequence of SEQ ID NO: 20. The human IGSF3 protein has a signal peptide (amino acids 1 to 19 of SEQ ID NO: 20), an extracellular domain (amino acids 20 to 1124 of SEQ ID NO: 20), a transmembrane domain (amino acids 1125 to 1145 of SEQ ID NO: 20), and a cytoplasmic domain (amino acids 1146 to 1194 of SEQ ID NO: 20).

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 28 to 613 of SEQ ID NO: 20. In some aspects, the IGSF3 protein lack one or more functional or structural domains, such as IgV.

In some aspects, a Scaffold X for the present disclosure comprises Integrin beta-1 (the ITGB1 protein), which is also known as Fibronectin receptor subunit beta, Glycoprotein IIa (GPIIA), VLA-4 subunit beta, or CD29, and is shown as the amino acid sequence of SEQ ID NO: 21. The human ITGB1 protein has a signal peptide (amino acids 1 to 20 of SEQ ID NO: 21), an extracellular domain (amino acids 21 to 728 of SEQ ID NO: 21), a transmembrane domain (amino acids 729 to 751 of SEQ ID NO: 21), and a cytoplasmic domain (amino acids 752 to 798 of SEQ ID NO: 21).

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 21 to 798 of SEQ ID NO: 21. In some aspects, the ITGB1 protein lack one or more functional or structural domains, such as IgV.

In other aspects, the Scaffold X comprises the ITGA4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 22 without the signal peptide (amino acids 1 to 33 of SEQ ID NO: 22). In some aspects, the ITGA4 protein lacks one or more functional or structural domains, such as IgV.

In other aspects, the Scaffold X comprises the SLC3A2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 23 without the signal peptide. In some aspects, the SLC3A2 protein lacks one or more functional or structural domains, such as IgV.

In other aspects, the Scaffold X comprises the ATP1A1 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 24 without the signal peptide. In some aspects, the ATP1A1 protein lacks one or more functional or structural domains, such as IgV.

In other aspects, the Scaffold X comprises the ATP1A2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 25 without the signal peptide. In some aspects, the ATP1A2 protein lacks one or more functional or structural domains, such as IgV.

In other aspects, the Scaffold X comprises the ATP1A3 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 26 without the signal peptide. In some aspects, the ATP1A3 protein lacks one or more functional or structural domains, such as IgV.

In other aspects, the Scaffold X comprises the ATP1A4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 27 without the signal peptide. In some aspects, the ATP1A4 protein lacks one or more functional or structural domains, such as IgV.

In other aspects, the Scaffold X comprises the ATP1A5 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 28 without the signal peptide. In some aspects, the ATP1A5 protein lacks one or more functional or structural domains, such as IgV.

In other aspects, the Scaffold X comprises the ATP2B1 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 29 without the signal peptide. In some aspects, the ATP2B1 protein lacks one or more functional or structural domains, such as IgV.

In other aspects, the Scaffold X comprises the ATP2B2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 30 without the signal peptide. In some aspects, the ATP2B2 protein lacks one or more functional or structural domains, such as IgV.

In other aspects, the Scaffold X comprises the ATP2B3 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 31 without the signal peptide. In some aspects, the ATP2B3 protein lacks one or more functional or structural domains, such as IgV.

In other aspects, the Scaffold X comprises the ATP2B4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 32 without the signal peptide. In some aspects, the ATP2B4 protein lacks one or more functional or structural domains, such as IgV.

In other aspects, the Scaffold X comprises the IGSF2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 34 without the signal peptide. In some aspects, the IGSF2 protein lacks one or more functional or structural domains, such as IgV.

Non-limiting examples of other Scaffold X proteins can be found at U.S. Pat. No. 10,195,290B1, issued Feb. 5, 2019, which is incorporated by reference in its entireties.

In some aspects, the sequence encodes a fragment of the scaffold moiety lacking at least 5, 10, 50, 100, 200, 300, 400, 500, 600, 700, or 800 amino acids from the N-terminus of the native protein. In some aspects, the sequence encodes a fragment of the scaffold moiety lacking at least 5, 10, 50, 100, 200, 300, 400, 500, 600, 700, or 800 amino acids from the C-terminus of the native protein. In some aspects, the sequence encodes a fragment of the scaffold moiety lacking at least 5, 10, 50, 100, 200, 300, 400, 500, 600, 700, or 800 amino acids from both the N-terminus and C-terminus of the native protein. In some aspects, the sequence encodes a fragment of the scaffold moiety lacking one or more functional or structural domains of the native protein.

In some aspects, the scaffold moieties, e.g., Scaffold X, e.g., a PTGFRN protein, are linked to one or more heterologous proteins. The one or more heterologous proteins can be linked to the N-terminus of the scaffold moieties. The one or more heterologous proteins can be linked to the C-terminus of the scaffold moieties. In some aspects, the one or more heterologous proteins are linked to both the N-terminus and the C-terminus of the scaffold moieties. In some aspects, the heterologous protein is a mammalian protein. In some aspects, the heterologous protein is a human protein.

In some aspects, Scaffold X can be used to link any moiety to the luminal surface and on the exterior surface of the EV, e.g., exosome, at the same time. For example, the PTGFRN polypeptide can be used to link a therapeutic molecule (e.g., an antigen), an adjuvant, and/or an immune modulator inside the lumen (e.g., on the luminal surface) in addition to the exterior surface of the EV, e.g., exosome. Therefore, in certain aspects, Scaffold X can be used for dual purposes, e.g., a therapeutic molecule (e.g., an antigen) on the luminal surface and an adjuvant or immune modulator on the exterior surface of the EV, e.g., exosome, a therapeutic molecule (e.g., an antigen) on the exterior surface of the EV, e.g., exosome, and the adjuvant or immune modulator on the luminal surface, an adjuvant on the luminal surface and an immune modulator on the exterior surface of the EV, e.g., exosome, or an immune modulator on the luminal surface and an adjuvant on the exterior surface of the EV, e.g., exosome.

### Scaffold Y-Engineered EVs, e.g., Exosomes

In some aspects, EVs, e.g., exosomes, of the present disclosure comprise an internal space (i.e., lumen) that is different from that of the naturally occurring EVs. For example, the EV can be changed such that the composition in the luminal surface of the EV, e.g., exosome, has the protein, lipid, or glycan content different from that of the naturally-occurring exosomes (e.g., comprises multiple exogenous biologically active molecules disclosed herein).

In some aspects, engineered EVs, e.g., exosomes, can be produced from a cell transformed with an exogenous sequence encoding a scaffold moiety (e.g., exosome proteins, e.g., Scaffold Y) or a modification or a fragment of the scaffold moiety that changes the composition or content of the luminal surface of the EV, e.g., exosome. Various modifications or fragments of the exosome protein that can be expressed on the luminal surface of the EV, e.g., exosome, can be used for the aspects of the present disclosure.

In some aspects, the exosome proteins that can change the luminal surface of the EVs, e.g., exosomes, include, but are not limited to, the myristoylated alanine rich Protein Kinase C substrate (MARCKS) protein, the myristoylated alanine rich Protein Kinase C substrate like 1 (MARCKSL1) protein, the brain acid soluble protein 1 (BASP1) protein, or any combination thereof. In certain aspects, EVs, e.g., exosomes, of the present disclosure comprise two or more (e.g., 2, 3, 4, 5 or more) of such exosome proteins.

In some aspects, Scaffold Y comprises the MARCKS protein (Uniprot accession no. P29966). The MARCKS protein is also known as protein kinase C substrate, 80 kDa protein, light chain. The full-length human MARCKS protein is 332 amino acids in length and comprises a calmodulin-binding domain at amino acid residues 152-176. In some aspects, Scaffold Y comprises the MARCKSL1 protein (Uniprot accession no. P49006). The MARCKSL1 protein is also known as MARCKS-like protein 1, and macrophage myristoylated alanine-rich C kinase substrate. The full-length human MARCKSL1 protein is 195 amino acids in length. The MARCKSL1 protein has an effector domain involved in lipid-binding and calmodulin-binding at amino acid residues 87-110. In some aspects, the Scaffold Y comprises the BASP1 protein (Uniprot accession number P80723). The BASP1 protein is also known as 22 kDa neuronal tissue-enriched acidic protein or neuronal axonal membrane protein NAP-22. The full-length human BASP1 protein sequence (isomer 1) is 227 amino acids in length. An isomer produced by an alternative splicing is missing amino acids 88 to 141 from SEQ ID NO: 49 (isomer 1). Table 2 provides the full-length sequences for the exemplary Scaffold Y disclosed herein (i.e., the MARCKS, MARCKSL1, and BASP1 proteins).

TABLE 2
Exemplary Scaffold Y Protein Sequences

| Protein | |
|---|---|
| The MARCKS | Sequence |
| protein | MGAQFSKTAA KGEAAAERPG EAAVASSPSK ANGQENGHVK VNGDASPAAA |
| (SEQ ID NO: 47) | ESGAKEELQA NGSAPAADKE EPAAAGSGAA SPSAAEKGEP AAAAAPEAGA |
| | SPVEKEAPAE GEAAEPGSPT AAEGEAASAA SSTSSPKAED GATPSPSNET |
| | PKKKKKRFSF KKSFKLSGFS FKKNKKEAGE GGEAEAPAAE GGKDEAAGGA |
| | AAAAAEAGAA SGEQAAAPGE EAAAGEEGAA GGDPQEAKPQ EAAVAPEKPP |
| | ASDETKAAEE PSKVEEKKAE EAGASAAACE APSAAGPGAP PEQEAAPAEE |
| | PAAAAASSAC AAPSQEAQPE CSPEAPPAEA AE |
| The MARCKSL1 | MGSQSSKAPR GDVTAEEAAG ASPAKANGQE NGHVKSNGDL SPKGEGESPP |
| protein | VNGTDEAAGA TGDAIEPAPP SQGAEAKGEV PPKETPKKKK KFSFKKPFKL |
| (SEQ ID NO: 48) | SGLSFKRNRK EGGGDSSASS PTEEEQEQGE IGACSDEGTA QEGKAAATPE |
| | SQEPQAKGAE ASAASEEEAG PQATEPSTPS GPESGPTPAS AEQNE |
| The BASP1 | MGGKLSKKKK GYNVNDEKAK EKDKKAEGAA TEEEGTPKES EPQAAAEPAE |
| protein | AKEGKEKPDQ DAEGKAEEKE GEKDAAAAKE EAPKAEPEKT EGAAEAKAEP |
| (SEQ ID NO: 49) | PKAPEQEQAA PGPAAGGEAP KAAEAAAAPA ESAAPAAGEE EAPSSTPKAQ |
| | TEAPAAPAAQ ETKSDGAPAS DSKPGSSEAA PSSKETPAAT PSKEEGEPKK |
| | GPAASAEEPK PVEAPAANSD QTVTVKE |

The mature BASP1 protein sequence is missing the first Met from SEQ ID NO: 49 and thus contains amino acids 2 to 227 of SEQ ID NO: 49. Similarly, the mature MARCKS and MARCKSL1 proteins also lack the first Met from SEQ ID NOs: 47 and 48, respectively. Accordingly, the mature MARCKS protein contains amino acids 2 to 332 of SEQ ID NO: 47. The mature MARCKSL1 protein contains amino acids 2 to 227 of SEQ ID NO: 48.

In other aspects, Scaffold Y useful for the present disclosure comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 2 to 227 of SEQ ID NO: 49. In other aspects, the Scaffold Y comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to any one of SEQ ID NOs: 50-155. In other aspects, a Scaffold Y useful for the present disclosure comprises the amino acid sequence of SEQ ID NO: 49, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, a Scaffold Y useful for the present disclosure comprises the amino acid sequence of any one of SEQ ID NOs: 50-155 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NOs: 50-155.

In some aspects, the protein sequence of any of SEQ ID NOs: 47-155 is sufficient to be a Scaffold Y for the present disclosure (e.g., scaffold moiety linked to a targeting moiety and/or a therapeutic molecule and/or an adjuvant and/or an immune modulator).

In some aspects, a Scaffold Y useful for the present disclosure comprises a peptide with the GXKLSKKK, where X is alanine or any other amino acid (SEQ ID NO: 376). In some aspects, an EV, e.g., exosome, comprises a peptide with sequence of (G)(π)(ξ)(Φ/π)(S/A/G/N)(+)(+), wherein each parenthetical position represents an amino acid, and wherein π is any amino acid selected from the group consisting of (Pro, Gly, Ala, Ser), ξ is any amino acid selected from the group consisting of (Asn, Gln, Ser, Thr, Asp, Glu, Lys, His, Arg), Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). In further aspects, an exosome described herein (e.g., engineered exosome) comprises a peptide with sequence of (G)(π)(X)(Φ/π)(π)(+)(+), wherein each parenthetical position represents an amino acid, and wherein π is any amino acid selected from the group consisting of (Pro, Gly, Ala, Ser), X is any amino acid, Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). See Aasland et al., FEBS Letters 513 (2002) 141-144 for amino acid nomenclature.

In other aspects, the Scaffold Y comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to any one of SEQ ID NO: 47-155.

Scaffold Y-engineered EVs, e.g., exosomes described herein can be produced from a cell transformed with a sequence set forth in SEQ ID NOs: 47-155.

In some aspects, the Scaffold Y protein useful for the present disclosure comprises an “N-terminus domain” (ND) and an “effector domain” (ED), wherein the ND and/or the ED are associated with the luminal surface of the EV, e.g., an exosome. In some aspects, the Scaffold Y protein useful for the present disclosure comprises an intracellular domain, a transmembrane domain, and an extracellular domain; wherein the intracellular domain comprises an “N-terminus domain” (ND) and an “effector domain” (ED), wherein the ND and/or the ED are associated with the luminal surface of the EV, e.g., an exosome. As used herein the term “associated with” refers to the interaction between a scaffold protein with the luminal surface of the EV, e.g., and exosome, that does not involve covalent linking to a membrane component. For example, the scaffolds useful for the present disclosure can be associated with the luminal surface of the EV, e.g., via a lipid anchor (e.g., myristic acid), and/or a polybasic domain that interacts electrostatically with the negatively charged head of membrane phospholipids. In other aspects, the Scaffold Y protein comprises an N-terminus domain (ND) and an effector domain (ED), wherein the ND is associated with the luminal surface of the EV and the ED are associated with the luminal surface of the EV by an ionic interaction, wherein the ED comprises at least two, at least three, at least four, at least five, at least six, or at least seven contiguous basic amino acids, e.g., lysines (Lys), in sequence.

In other aspects, the Scaffold Y protein comprises an N-terminus domain (ND) and an effector domain (ED), wherein the ND is associated with the luminal surface of the EV, e.g., exosome, and the ED is associated with the luminal surface of the EV by an ionic interaction, wherein the ED comprises at least two, at least three, at least four, at least five, at least six, or at least seven contiguous basic amino acids, e.g., lysines (Lys), in sequence.

In some aspects, the ND is associated with the luminal surface of the EV, e.g., an exosome, via lipidation, e.g., via myristoylation. In some aspects, the ND has Gly at the N terminus. In some aspects, the N-terminal Gly is myristoylated.

In some aspects, the ED is associated with the luminal surface of the EV, e.g., an exosome, by an ionic interaction. In some aspects, the ED is associated with the luminal surface of the EV, e.g., an exosome, by an electrostatic interaction, in particular, an attractive electrostatic interaction.

In some aspects, the ED comprises (i) a basic amino acid (e.g., lysine), or (ii) two or more basic amino acids (e.g., lysine) next to each other in a polypeptide sequence. In some aspects, the basic amino acid is lysine (Lys; K), arginine (Arg, R), or Histidine (His, H). In some aspects, the basic amino acid is (Lys)n, wherein n is an integer between 1 and 10.

In other aspects, the ED comprises at least a lysine and the ND comprises a lysine at the C terminus if the N terminus of the ED is directly linked to lysine at the C terminus of the ND, i.e., the lysine is in the N terminus of the ED and is fused to the lysine in the C terminus of the ND. In other aspects, the ED comprises at least two lysines, at least three lysines, at least four lysines, at least five lysines, at least six lysines, or at least seven lysines when the N terminus of the ED is linked to the C terminus of the ND by a linker, e.g., one or more amino acids.

In some aspects, the ED comprises K, KK, KKK, KKKK (SEQ ID NO: 205), KKKKK (SEQ ID NO: 206), R, RR, RRR, RRRR (SEQ ID NO: 207); RRRRR (SEQ ID NO: 208), KR, RK, KKR, KRK, RKK, KRR, RRK, (K/R)(K/R)(K/R)(K/R) (SEQ ID NO: 209), (K/R)(K/R)(K/R)(K/R)(K/R) (SEQ ID NO: 210), or any combination thereof. In some aspects, the ED comprises KK, KKK, KKKK (SEQ ID NO: 205), KKKKK (SEQ ID NO: 206), or any combination thereof. In some aspects, the ND comprises the amino acid sequence as set forth in G:X2:X3:X4:X5:X6, wherein G represents Gly; wherein “:” represents a peptide bond; wherein each of the X2 to the X6 independently represents an amino acid; and wherein the X6 represents a basic amino acid. In some aspects, the X6 amino acid is selected is selected from the group consisting of Lys, Arg, and His. In some aspects, the X5 amino acid is selected from the group consisting of Pro, Gly, Ala, and Ser. In some aspects, the X2 amino acid is selected from the group consisting of Pro, Gly, Ala, and Ser. In some aspects, the X4 is selected from the group consisting of Pro, Gly, Ala, Ser, Val, Ile, Leu, Phe, Trp, Tyr, Gln, and Met.

In some aspects, the Scaffold Y protein comprises an N-terminus domain (ND) and an effector domain (ED), wherein the ND comprises the amino acid sequence as set forth in G:X2:X3:X4:X5:X6, wherein G represents Gly; wherein “:” represents a peptide bond; wherein each of the X2 to the X6 is independently an amino acid; wherein the X6 comprises a basic amino acid, and wherein the ED is linked to X6 by a peptide bond and comprises at least one lysine at the N terminus of the ED.

In some aspects, the ND of the Scaffold Y protein comprises the amino acid sequence of G:X2:X3:X4:X5:X6, wherein G represents Gly; “:” represents a peptide bond; the X2 represents an amino acid selected from the group consisting of Pro, Gly, Ala, and Ser; the X3 represents any amino acid; the X4 represents an amino acid selected from the group consisting of Pro, Gly, Ala, Ser, Val, Ile, Leu, Phe, Trp, Tyr, Gln, and Met; the X5 represents an amino acid selected from the group consisting of Pro, Gly, Ala, and Ser; and the X6 represents an amino acid selected from the group consisting of Lys, Arg, and His.

In some aspects, the X3 amino acid is selected from the group consisting of Asn, Gln, Ser, Thr, Asp, Glu, Lys, His, and Arg.

In some aspects, the ND and ED are joined by a linker. In some aspects, the linker comprises one or more amino acids. In some aspects, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. Generally, linkers provide flexibility or prevent/ameliorate steric hindrances. Linkers are not typically cleaved; however, in certain aspects, such cleavage can be desirable. Accordingly, in some aspects a linker can comprise one or more protease-cleavable sites, which can be located within the sequence of the linker or flanking the linker at either end of the linker sequence. When the ND and ED are joined by a linker, the ED comprise at least two lysines, at least three lysines, at least four lysines, at least five lysines, at least six lysines, or at least seven lysines.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the linker is a glycine/serine linker. In some aspects, the peptide linker is glycine/serine linker according to the formula [(Gly)n-Ser]m where n is any integer from 1 to 100 and m is any integer from 1 to 100. In other aspects, the glycine/serine linker is according to the formula [(Gly)x-Sery]z wherein x in an integer from 1 to 4, y is 0 or 1, and z is an integers from 1 to 50. In some aspects, the peptide linker comprises the sequence Gn, where n can be an integer from 1 to 100. In some aspects, the peptide linker can comprise the sequence (GlyAla)n, wherein n is an integer between 1 and 100. In other aspects, the peptide linker can comprise the sequence (GlyGlySer)n, wherein n is an integer between 1 and 100.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

In other aspects, the peptide linker can comprise non-naturally occurring amino acids. In yet other aspects, the peptide linker can comprise naturally occurring amino acids occurring in a linear sequence that does not occur in nature. In still other aspects, the peptide linker can comprise a naturally occurring polypeptide sequence.

The present disclosure also provides an isolated extracellular vesicle (EV), e.g., an exosome, comprising a targeting moiety and an additional exogenous biologically active molecule (e.g., a therapeutic molecule, an adjuvant, and/or an immune modulator) linked to a Scaffold Y protein, wherein the Scaffold Y protein comprises ND-ED, wherein: ND comprises G:X2:X3:X4:X5:X6; wherein: G represents Gly; “:” represents a peptide bond; X2 represents an amino acid selected from the group consisting of Pro, Gly, Ala, and Ser; X3 represents any amino acid; X4 represents an amino acid selected from the group consisting of Pro, Gly, Ala, Ser, Val, Ile, Leu, Phe, Trp, Tyr, Glu, and Met; X5 represents an amino acid selected from the group consisting of Pro, Gly, Ala, and Ser; X6 represents an amino acid selected from the group consisting of Lys, Arg, and His; “-” represents an optional linker; and ED is an effector domain comprising (i) at least two contiguous lysines (Lys), which is linked to the X6 by a peptide bond or one or more amino acids or (ii) at least one lysine, which is directly linked to the X6 by a peptide bond.

In some aspects, the X2 amino acid is selected from the group consisting of Gly and Ala. In some aspects, the X3 amino acid is Lys. In some aspects, the X4 amino acid is Leu or Glu. In some aspects, the X5 amino acid is selected from the group consisting of Ser and Ala. In some aspects, the X6 amino acid is Lys. In some aspects, the X2 amino acid is Gly, Ala, or Ser; the X3 amino acid is Lys or Glu; the X4 amino acid is Leu, Phe, Ser, or Glu; the X5 amino acid is Ser or Ala; and X6 amino acid is Lys. In some aspects, the “-” linker comprises a peptide bond or one or more amino acids.

In some aspects, the ED in the scaffold protein comprises Lys (K), KK, KKK, KKKK (SEQ ID NO: 205), KKKKK (SEQ ID NO: 206), Arg (R), RR, RRR, RRRR (SEQ ID NO: 207); RRRRR (SEQ ID NO: 208), KR, RK, KKR, KRK, RKK, KRR, RRK, (K/R)(K/R)(K/R)(K/R) (SEQ ID NO: 209), (K/R)(K/R)(K/R)(K/R)(K/R) (SEQ ID NO: 210), or any combination thereof.

In some aspects, the Scaffold Y protein comprises an amino acid sequence selected from the group consisting of (i) GGKLSKK (SEQ ID NO: 211), (ii) GAKLSKK (SEQ ID NO: 212), (iii) GGKQSKK (SEQ ID NO: 213), (iv) GGKLAKK (SEQ ID NO: 214), or (v) any combination thereof.

In some aspects, the ND in the Scaffold Y protein comprises an amino acid sequence selected from the group consisting of (i) GGKLSK (SEQ ID NO: 215), (ii) GAKLSK (SEQ ID NO: 216), (iii) GGKQSK (SEQ ID NO: 217), (iv) GGKLAK (SEQ ID NO: 218), or (v) any combination thereof and the ED in the scaffold protein comprises K, KK, KKK, KKKG (SEQ ID NO: 219), KKKGY (SEQ ID NO: 220), KKKGYN (SEQ ID NO: 221), KKKGYNV (SEQ ID NO: 222), KKKGYNVN (SEQ ID NO: 223), KKKGYS (SEQ ID NO: 224), KKKGYG (SEQ ID NO: 225), KKKGYGG (SEQ ID NO: 226), KKKGS (SEQ ID NO: 227), KKKGSG (SEQ ID NO: 228), KKKGSGS (SEQ ID NO: 229), KKKS (SEQ ID NO: 230), KKKSG (SEQ ID NO: 231), KKKSGG (SEQ ID NO: 232), KKKSGGS (SEQ ID NO: 233), KKKSGGSG (SEQ ID NO: 234), KKSGGSGG (SEQ ID NO: 235), KKKSGGSGGS (SEQ ID NO: 236), KRFSFKKS (SEQ ID NO: 237).

In some aspects, the polypeptide sequence of a Scaffold Y protein useful for the present disclosure consists of an amino acid sequence selected from the group consisting of (i) GGKLSKK (SEQ ID NO: 211), (ii) GAKLSKK (SEQ ID NO: 212), (iii) GGKQSKK (SEQ ID NO: 213), (iv) GGKLAKK (SEQ ID NO: 214), or (v) any combination thereof.

In some aspects, the Scaffold Y protein comprises an amino acid sequence selected from the group consisting of (i) GGKLSKKK (SEQ ID NO: 238), (ii) GGKLSKKS (SEQ ID NO: 239), (iii) GAKLSKKK (SEQ ID NO: 240), (iv) GAKLSKKS (SEQ ID NO: 241), (v) GGKQSKKK (SEQ ID NO: 242), (vi) GGKQSKKS (SEQ ID NO: 243), (vii) GGKLAKKK (SEQ ID NO: 244), (viii) GGKLAKKS (SEQ ID NO: 245), and (ix) any combination thereof.

In some aspects, the polypeptide sequence of a Scaffold Y protein useful for the present disclosure consists of an amino acid sequence selected from the group consisting of (i) GGKLSKKK (SEQ ID NO: 238), (ii) GGKLSKKS (SEQ ID NO: 239), (iii) GAKLSKKK (SEQ ID NO: 240), (iv) GAKLSKKS (SEQ ID NO: 241), (v) GGKQSKKK (SEQ ID NO: 242), (vi) GGKQSKKS (SEQ ID NO: 243), (vii) GGKLAKKK (SEQ ID NO: 244), (viii) GGKLAKKS (SEQ ID NO: 245), and (ix) any combination thereof.

In some aspects, the Scaffold Y protein is at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least 31, at least about 32, at least about 33, at least about 34, at least about 35, at least about 36, at least about 37, at least about 38, at least about 39, at least about 39, at least about 40, at least about 41, at least about 42, at least about 43, at least about 44, at least about 50, at least about 46, at least about 47, at least about 48, at least about 49, at least about 50, at least about 55, at least about 60, at least about 65, at least about 70, at least about 75, at least about 80, at least 85, at least about 90, at least about 95, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, at least about 195, at least about 200, at least about 205, at least about 210, at least about 215, at least about 220, at least about 225, at least about 230, at least about 235, at least about 240, at least about 245, at least about 250, at least about 255, at least about 260, at least about 265, at least about 270, at least about 275, at least about 280, at least about 285, at least about 290, at least about 295, at least about 300, at least about 305, at least about 310, at least about 315, at least about 320, at least about 325, at least about 330, at least about 335, at least about 340, at least about 345, or at least about 350 amino acids in length.

In some aspects, the Scaffold Y protein is between about 5 and about 10, between about 10 and about 20, between about 20 and about 30, between about 30 and about 40, between about 40 and about 50, between about 50 and about 60, between about 60 and about 70, between about 70 and about 80, between about 80 and about 90, between about 90 and about 100, between about 100 and about 110, between about 110 and about 120, between about 120 and about 130, between about 130 and about 140, between about 140 and about 150, between about 150 and about 160, between about 160 and about 170, between about 170 and about 180, between about 180 and about 190, between about 190 and about 200, between about 200 and about 210, between about 210 and about 220, between about 220 and about 230, between about 230 and about 240, between about 240 and about 250, between about 250 and about 260, between about 260 and about 270, between about 270 and about 280, between about 280 and about 290, between about 290 and about 300, between about 300 and about 310, between about 310 and about 320, between about 320 and about 330, between about 330 and about 340, or between about 340 and about 250 amino acids in length.

In some aspects, the Scaffold Y protein comprises (i) GGKLSKKKKGYNVN (SEQ ID NO: 246), (ii) GAKLSKKKKGYNVN (SEQ ID NO: 247), (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254), (x) GGKLSKSGGSGGSV (SEQ ID NO: 255), or (xi) GAKKSKKRFSFKKS (SEQ ID NO: 256).

In some aspects, the polypeptide sequence of a Scaffold Y protein useful for the present disclosure consists of (i) GGKLSKKKKGYNVN (SEQ ID NO: 246), (ii) GAKLSKKKKGYNVN (SEQ ID NO: 247), (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254), (x) GGKLSKSGGSGGSV (SEQ ID NO: 255), or (xi) GAKKSKKRFSFKKS (SEQ ID NO: 256\hat{ }#).

Non-limiting examples of the Scaffold Y protein useful for the present disclosure are listed below. In some aspects, the Scaffold Y protein comprises an amino acid sequence set forth in Table 3. In some aspects, the Scaffold Y protein consists of an amino acid sequence set forth in Table 3.

TABLE 3

| SEQ ID NO: | Scaffold Protein: GX2X3X4X5X6-ED |
|---|---|
| 257 | GGKLSKKKKGYNVNDEKAKEKDKKAEGAA |
| 258 | GGKLSKKKKGYNVNDEKAKEKDKKAEGA |
| 259 | GGKLSKKKKGYNVNDEKAKEKDKKAEG |
| 260 | GGKLSKKKKGYNVNDEKAKEKDKKAE |
| 261 | GGKLSKKKKGYNVNDEKAKEKDKKA |
| 262 | GGKLSKKKKGYNVNDEKAKEKDKK |
| 263 | GGKLSKKKKGYNVNDEKAKEKDK |
| 264 | GGKLSKKKKGYNVNDEKAKEKD |
| 265 | GGKLSKKKKGYNVNDEKAKEK |
| 266 | GGKLSKKKKGYNVNDEKAKE |
| 267 | GGKLSKKKKGYNVNDEKAK |
| 268 | GGKLSKKKKGYNVNDEKA |
| 269 | GGKLSKKKKGYNVNDEK |
| 270 | GGKLSKKKKGYNVNDE |
| 271 | GGKLSKKKKGYNVND |
| 246 | GGKLSKKKKGYNVN |
| 272 | GGKLSKKKKGYNV |
| 273 | GGKLSKKKKGYN |
| 274 | GGKLSKKKKGY |
| 275 | GGKLSKKKKG |
| 276 | GGKLSKKKK |
| 238 | GGKLSKKK |
| 211 | GGKLSKK |
| 300 | GAKKSKKRFSFKKSFKLSGFSFKKNKKEA |
| 277 | GAKKSKKRFSFKKSFKLSGFSFKKNKKE |
| 278 | GAKKSKKRFSFKKSFKLSGFSFKKNKK |
| 279 | GAKKSKKRFSFKKSFKLSGFSFKKNK |
| 280 | GAKKSKKRFSFKKSFKLSGFSFKKN |
| 281 | GAKKSKKRFSFKKSFKLSGFSFKK |
| 282 | GAKKSKKRFSFKKSFKLSGFSFK |
| 283 | GAKKSKKRFSFKKSFKLSGFSF |
| 284 | GAKKSKKRFSFKKSFKLSGES |
| 285 | GAKKSKKRFSFKKSFKLSGF |
| 286 | GAKKSKKRFSFKKSFKLSG |
| 287 | GAKKSKKRFSFKKSFKLS |
| 288 | GAKKSKKRFSFKKSFKL |
| 289 | GAKKSKKRFSFKKSFK |
| 290 | GAKKSKKRFSFKKSF |
| 291 | GAKKSKKRFSFKKS |
| 292 | GAKKSKKRFSFKK |
| 293 | GAKKSKKRFSFK |
| 294 | GAKKSKKRFSF |
| 295 | GAKKSKKRFS |
| 296 | GAKKSKKRF |
| 297 | GAKKSKKR |
| 298 | GAKKSKK |
| 301 | GAKKAKKRFSFKKSFKLSGFSFKKNKKEA |
| 348 | GAKKAKKRFSFKKSFKLSGFSFKKNKKE |
| 349 | GAKKAKKRFSFKKSFKLSGFSFKKNKK |
| 350 | GAKKAKKRFSFKKSFKLSGFSFKKNK |
| 351 | GAKKAKKRFSFKKSFKLSGFSFKKN |
| 352 | GAKKAKKRFSFKKSFKLSGESFKK |
| 353 | GAKKAKKRFSFKKSFKLSGESEK |
| 354 | GAKKAKKRFSFKKSFKLSGESE |
| 355 | GAKKAKKRFSFKKSFKLSGES |
| 356 | GAKKAKKRFSFKKSFKLSGF |
| 357 | GAKKAKKRFSFKKSFKLSG |
| 358 | GAKKAKKRFSFKKSFKLS |
| 359 | GAKKAKKRFSFKKSFKL |
| 360 | GAKKAKKRFSFKKSFK |
| 361 | GAKKAKKRFSFKKSF |
| 362 | GAKKAKKRESFKKS |
| 363 | GAKKAKKRFSFKK |
| 364 | GAKKAKKRFSFK |
| 365 | GAKKAKKRFSF |
| 366 | GAKKAKKRES |
| 367 | GAKKAKKRF |
| 368 | GAKKAKKR |
| 369 | GAKKAKK |
| 302 | GAQESKKKKKKRFSFKKSFKLSGFSFKK |
| 303 | GAQESKKKKKKRFSFKKSFKLSGESEK |
| 304 | GAQESKKKKKKRFSFKKSFKLSGFSF |
| 305 | GAQESKKKKKKRFSFKKSFKLSGFS |
| 306 | GAQESKKKKKKRFSFKKSFKLSGE |
| 307 | GAQESKKKKKKRFSFKKSFKLSG |
| 308 | GAQESKKKKKKRFSFKKSFKLS |
| 309 | GAQESKKKKKKRFSFKKSFKL |
| 310 | GAQESKKKKKKRESFKKSFK |
| 311 | GAQESKKKKKKRFSFKKSF |
| 312 | GAQESKKKKKKRFSFKKS |
| 313 | GAQESKKKKKKRFSFKK |
| 314 | GAQESKKKKKKRFSFK |
| 315 | GAQESKKKKKKRFSF |
| 316 | GAQESKKKKKKRFS |
| 317 | GAQESKKKKKKRF |
| 318 | GAQESKKKKKKR |
| 319 | GAQESKKKKKK |
| 320 | GAQESKKKKK |
| 321 | GAQESKKKK |
| 322 | GAQESKKK |
| 323 | GAQESKK |
| 324 | GSQSSKKKKKKFSFKKPFKLSGLSFKRNRK |
| 325 | GSQSSKKKKKKFSFKKPFKLSGLSFKRNR |
| 326 | GSQSSKKKKKKFSFKKPFKLSGLSFKRN |
| 327 | GSQSSKKKKKKFSFKKPFKLSGLSFKR |
| 328 | GSQSSKKKKKKFSFKKPFKLSGLSFK |
| 329 | GSQSSKKKKKKFSFKKPFKLSGLSF |
| 330 | GSQSSKKKKKKFSFKKPFKLSGLS |
| 331 | GSQSSKKKKKKFSFKKPFKLSGL |
| 332 | GSQSSKKKKKKFSFKKPFKLSG |
| 333 | GSQSSKKKKKKFSFKKPFKLS |
| 334 | GSQSSKKKKKKFSFKKPFKL |
| 335 | GSQSSKKKKKKFSFKKPFK |
| 336 | GSQSSKKKKKKFSFKKPF |
| 337 | GSQSSKKKKKKFSFKKP |
| 338 | GSQSSKKKKKKFSFKK |
| 339 | GSQSSKKKKKKFSFK |
| 340 | GSQSSKKKKKKFSF |
| 341 | GSQSSKKKKKKES |
| 342 | GSQSSKKKKKKF |
| 343 | GSQSSKKKKKK |
| 344 | GSQSSKKKKK |
| 345 | GSQSSKKKK |
| 346 | GSQSSKKK |
| 347 | GSQSSKK |

In some aspects, the Scaffold Y protein useful for the present disclosure does not contain an N-terminal Met. In some aspects, the Scaffold Y protein comprises a lipidated amino acid, e.g., a myristoylated amino acid, at the N-terminus of the scaffold protein, which functions as a lipid anchor. In some aspects, the amino acid residue at the N-terminus of the scaffold protein is Gly. The presence of an N-terminal Gly is an absolute requirement for N-myristoylation. In some aspects, the amino acid residue at the N-terminus of the scaffold protein is synthetic. In some aspects, the amino acid residue at the N-terminus of the scaffold protein is a glycine analog, e.g., allylglycine, butylglycine, or propargylglycine.

In other aspects, the lipid anchor can be any lipid anchor known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached posttranslationally to internal serine/threonine or tyrosine residues via a hydroxyester linkage. Membrane anchors known in the art are presented in the following table:

| Modification | Modifying Group |
|---|---|
| S-Palmitoylation | 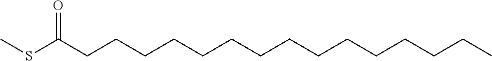 [SMILES: CCCCCCCCCCCCCCCC(=O)SC] |
| N-Palmitoylation | 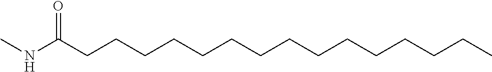 [SMILES: CCCCCCCCCCCCCCCC(=O)NC] |
| N-Myristoylation | 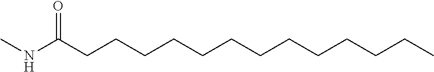 [SMILES: CCCCCCCCCCCCCC(=O)NC] |
| O-Acylation | 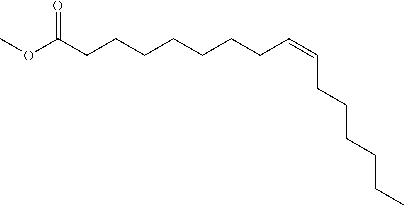 [SMILES: CCCCCC/C=C\CCCCCCCC(=O)OC] |
| Farnesylation | 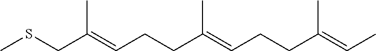 [SMILES: C/C=C(\C)CC/C=C(\C)CC/C=C(\C)CSC] |
| Geranylgeranylation | 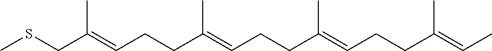 [SMILES: C/C=C(\C)CC/C=C(\C)CC/C=C(\C)CC/C=C(\C)CSC] |
| Cholesterol | 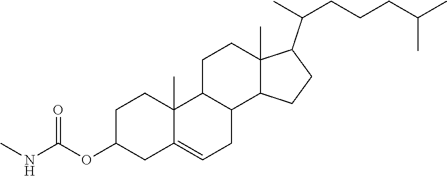 [SMILES: CNC(=O)OC1CCC2(C)C(=CCC3C2CCC2(C)C(C(C)CCCC(C)C)CCC32)C1] |

### Linkers

As described supra, extracellular vesicles (EVs) of the present disclosure (e.g., exosomes and nanovesicles) can comprises one or more linkers that link one or more exogenous biologically active molecules disclosed herein (e.g., targeting moiety, therapeutic molecule (e.g., antigen), adjuvant, or immune modulator) to the EVs (e.g., to the exterior surface or on the luminal surface). In some aspects, the one or more exogenous biologically active molecules (e.g., targeting moiety, therapeutic molecule, adjuvant, or immune modulator) are linked to the EVs directly or via one or more scaffold moieties (e.g., Scaffold X or Scaffold Y). For example, in certain aspects, one or more exogenous biologically active molecules are linked to the exterior surface of an exosome via Scaffold X. In further aspects, one or more exogenous biologically active molecules are linked to the luminal surface of an exosome via Scaffold X or Scaffold Y. The linker can be any chemical moiety known in the art.

As used herein, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present, each of the linkers can be the same or different. Generally, linkers provide flexibility or prevent/ameliorate steric hindrances. Linkers are not typically cleaved; however in certain aspects, such cleavage can be desirable. Accordingly, in some aspects, a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

Linkers can be susceptible to cleavage (“cleavable linker”) thereby facilitating release of the exogenous biologically active molecule (e.g., targeting moiety, therapeutic molecule, adjuvant, or immune modulator).

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the linker comprises a non-cleavable linker.

### III. Producer Cell for Production of Engineered Exosomes

EVs, e.g., exosomes, of the present disclosure can be produced from a cell grown in vitro or a body fluid of a subject. When exosomes are produced from in vitro cell culture, various producer cells, e.g., HEK293 cells, CHO cells, and MSCs, can be used. In certain aspects, a producer cell is not a dendritic cell, macrophage, B cell, mast cell, neutrophil, Kupffer-Browicz cell, cell derived from any of these cells, or any combination thereof.

The producer cell can be genetically modified to comprise one or more exogenous sequences (e.g., encoding one or more exogenous biologically active molecules disclosed herein, e.g., a targeting moiety, therapeutic molecule (e.g., an antigen), adjuvant, or immune modulator) to produce exosomes described herein. The genetically-modified producer cell can contain the exogenous sequences by transient or stable transformation. The exogenous sequences can be transformed as a plasmid. The exogenous sequences can be stably integrated into a genomic sequence of the producer cell, at a targeted site or in a random site. In some aspects, a stable cell line is generated for production of EVs disclosed herein, e.g., exosomes.

The exogenous sequences can be inserted into a genomic sequence of the producer cell, located within, upstream (5′-end) or downstream (3′-end) of an endogenous sequence encoding an exosome protein. Various methods known in the art can be used for the introduction of the exogenous sequences into the producer cell. For example, cells modified using various gene editing methods (e.g., methods using a homologous recombination, transposon-mediated system, loxP-Cre system, CRISPR/Cas9 or TALEN) are within the scope of the present disclosure.

The exogenous sequences can comprise a sequence encoding a scaffold moiety disclosed herein or a fragment or variant thereof. An extra copy of the sequence encoding a scaffold moiety can be introduced to produce an exosome described herein (e.g., having a higher density of a scaffold moiety or expressing multiple different scaffold moieties on the surface or on the luminal surface of the EV, e.g., exosome). Exogenous sequences encoding a modification or a fragment of a scaffold moiety can be introduced to produce a lumen-engineered and/or surface-engineered exosome containing the modification or the fragment of the scaffold moiety.

In some aspects, a producer cell can be modified, e.g., transfected, with one or more vectors encoding one or more scaffold moieties linked to exogenous biologically active molecules described herein (e.g., targeting moiety, therapeutic molecule (e.g., an antigen), an adjuvant, and/or an immune modulator).

In some aspects, a producer cell disclosed herein is further modified to comprise an additional exogenous sequence. For example, an additional exogenous sequence can be introduced to modulate endogenous gene expression, or produce an exosome including a certain polypeptide as a payload (e.g., antigen). In some aspects, the producer cell is modified to comprise two exogenous sequences, one encoding a scaffold moiety (e.g., Scaffold X and/or Scaffold Y), or a variant or a fragment thereof, and the other encoding a payload. In certain aspects, the producer cell can be further modified to comprise an additional exogenous sequence conferring additional functionalities to exosomes (e.g., adjuvants, immune modulators, or targeting moieties). In some aspects, the producer cell is modified to comprise two exogenous sequences, one encoding a scaffold moiety disclosed herein, or a variant or a fragment thereof, and the other encoding a protein conferring the additional functionalities to exosomes (e.g., adjuvants, immune modulators, or targeting moieties). In some aspects, the producer cell is further modified to comprise one, two, three, four, five, six, seven, eight, nine, or ten or more additional exogenous sequences.

In some aspects, EVs, e.g., exosomes, of the present disclosure (e.g., surface-engineered and/or lumen-engineered exosomes) can be produced from a cell transformed with a sequence encoding a full-length, mature scaffold moiety disclosed herein or a scaffold moiety linked to a targeting moiety, a therapeutic molecule (e.g., an antigen), an adjuvant, and/or an immune modulator. Any of the scaffold moieties described herein can be expressed from a plasmid, an exogenous sequence inserted into the genome or other exogenous nucleic acid, such as a synthetic messenger RNA (mRNA).

### IV. Pharmaceutical Compositions

Provided herein are pharmaceutical compositions comprising an EV, e.g., exosome, of the present disclosure having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents and an exosome described herein. In certain aspects, the EVs, e.g., exosomes, are co-administered with of one or more additional therapeutic agents, in a pharmaceutically acceptable carrier. In some aspects, the pharmaceutical composition comprising the EV, e.g., exosome is administered prior to administration of the additional therapeutic agents. In other aspects, the pharmaceutical composition comprising the EV, e.g., exosome is administered after the administration of the additional therapeutic agents. In further aspects, the pharmaceutical composition comprising the EV, e.g., exosome is administered concurrently with the additional therapeutic agents.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration. The EVs, e.g., exosomes, can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition comprising exosomes is administered intravenously, e.g. by injection. The EVs, e.g., exosomes, can optionally be administered in combination with other therapeutic agents that are at least partly effective in treating the disease, disorder or condition for which the EVs, e.g., exosomes, are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as manitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs, e.g., exosomes, in an effective amount and in an appropriate solvent with one or a combination of ingredients enumerated herein, as desired. Generally, dispersions are prepared by incorporating the EVs, e.g., exosomes, into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The EVs, e.g., exosomes, can be administered in the form of a depot injection or implant preparation which can be formulated in such a manner to permit a sustained or pulsatile release of the EV, e.g., exosomes.

Systemic administration of compositions comprising exosomes can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising exosomes is administered intravenously into a subject that would benefit from the pharmaceutical composition. In certain other aspects, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

In certain aspects, the pharmaceutical composition comprising exosomes is administered as a liquid suspension. In certain aspects, the pharmaceutical composition is administered as a formulation that is capable of forming a depot following administration. In certain preferred aspects, the depot slowly releases the EVs, e.g., exosomes, into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the EVs, e.g., exosomes, described herein and optionally a pharmaceutically active or therapeutic agent. The therapeutic agent can be a biological agent, a small molecule agent, or a nucleic acid agent.

Dosage forms are provided that comprise a pharmaceutical composition comprising the EVs, e.g., exosomes, described herein. In some aspects, the dosage form is formulated as a liquid suspension for intravenous injection. In some aspects, the dosage form is formulated as a liquid suspension for intratumoral injection.

In certain aspects, the preparation of exosomes is subjected to radiation, e.g., X rays, gamma rays, beta particles, alpha particles, neutrons, protons, elemental nuclei, UV rays in order to damage residual replication-competent nucleic acids.

In certain aspects, the preparation of exosomes is subjected to gamma irradiation using an irradiation dose of more than 1, 5, 10, 15, 20, 25, 30, 35, 40, 50, 60, 70, 80, 90, 100, or more than 100 kGy.

In certain aspects, the preparation of exosomes is subjected to X-ray irradiation using an irradiation dose of more than 0.1, 0.5, 1, 5, 10, 15, 20, 25, 30, 35, 40, 50, 60, 70, 80, 90, 100, 200, 300, 400, 500, 600, 700, 800, 900, 1000, 2000, 3000, 4000, 5000, 6000, 7000, 8000, 9000, 10000, or greater than 10000 mSv.

### V. Kits

Also provided herein are kits comprising one or more exosomes described herein. In some aspects, provided herein is a pharmaceutical pack or kit comprising one or more containers filled with one or more of the ingredients of the pharmaceutical compositions described herein, such as one or more exosomes provided herein, optional an instruction for use. In some aspects, the kits contain a pharmaceutical composition described herein and any prophylactic or therapeutic agent, such as those described herein.

### VI. Methods of Producing Exosomes

In some aspects, the present disclosure is also directed to methods of producing exosomes described herein. In some aspects, the method comprises: obtaining the EV, e.g., exosome, from a producer cell, wherein the producer cell contains two or more components of the EV, e.g., exosome (e.g., (i) therapeutic molecule and adjuvant, (ii) therapeutic molecule and immune modulator, or (iii) therapeutic molecule, adjuvant, and immune modulator); and optionally isolating the obtained EV, e.g., exosome. In some aspects, the method comprises: modifying a producer cell by introducing two or more components of an exosome disclosed herein (e.g., (i) therapeutic molecule and adjuvant, (ii) therapeutic molecule and immune modulator, or (iii) therapeutic molecule, adjuvant, and immune modulator); obtaining the EV, e.g., exosome from the modified producer cell; and optionally isolating the obtained EV, e.g., exosome. In further aspects, the method comprises: obtaining an exosome from a producer cell; isolating the obtained exosome; and modifying the isolated exosome (e.g., by inserting multiple exogenous biologically active molecules, e.g., therapeutic molecules, adjuvants, immune modulators, and/or targeting moieties). In certain aspects, the method further comprises formulating the isolated exosome into a pharmaceutical composition.

### Methods of Modifying a Producer Cell

As described supra, in some aspects, a method of producing an exosome comprises modifying a producer cell with multiple (e.g., two or more) exogenous biologically active molecules described herein (e.g., therapeutic molecule, adjuvant, immune modulator, and/or targeting moiety). In some aspects, a producer cell disclosed herein can be further modified with a scaffold moiety disclosed herein (e.g., Scaffold X or Scaffold Y).

In some aspects, the producer cell can be a mammalian cell line, a plant cell line, an insect cell line, a fungi cell line, or a prokaryotic cell line. In certain aspects, the producer cell is a mammalian cell line. Non-limiting examples of mammalian cell lines include: a human embryonic kidney (HEK) cell line, a Chinese hamster ovary (CHO) cell line, an HT-1080 cell line, a HeLa cell line, a PERC-6 cell line, a CEVEC cell line, a fibroblast cell line, an amniocyte cell line, an epithelial cell line, a mesenchymal stem cell (MSC) cell line, and combinations thereof. In certain aspects, the mammalian cell line comprises HEK-293 cells, BJ human foreskin fibroblast cells, fHDF fibroblast cells, AGE.HN® neuronal precursor cells, CAP® amniocyte cells, adipose mesenchymal stem cells, RPTEC/TERT1 cells, or combinations thereof. In some aspects, the producer cell is a primary cell. In certain aspects, the primary cell can be a primary mammalian cell, a primary plant cell, a primary insect cell, a primary fungi cell, or a primary prokaryotic cell.

In some aspects, the producer cell is not an immune cell, such as an antigen presenting cell, a T cell, a B cell, a natural killer cell (NK cell), a macrophage, a T helper cell, or a regulatory T cell (Treg cell). In other aspects, the producer cell is not an antigen presenting cell (e.g., dendritic cells, macrophages, B cells, mast cells, neutrophils, Kupffer-Browicz cell, or a cell derived from any such cells).

In some aspects, the multiple exogenous biologically active molecules used to modify a producer cell can be a transgene or mRNA, and introduced into the producer cell by transfection, viral transduction, electroporation, extrusion, sonication, cell fusion, or other methods that are known to the skilled in the art.

In some aspects, the multiple exogenous biologically active molecules are introduced to the producer cell by transfection. In some aspects, the multiple exogenous biologically active molecules can be introduced into suitable producer cells using synthetic macromolecules, such as cationic lipids and polymers (Papapetrou et al., Gene Therapy 12: S118-S130 (2005)). In some aspects, the cationic lipids form complexes with the multiple exogenous biologically active molecules through charge interactions. In some of these aspects, the positively charged complexes bind to the negatively charged cell surface and are taken up by the cell by endocytosis. In some other aspects, a cationic polymer can be used to transfect producer cells. In some of these aspects, the cationic polymer is polyethylenimine (PEI). In certain aspects, chemicals such as calcium phosphate, cyclodextrin, or polybrene, can be used to introduce the multiple exogenous biologically active molecules to the producer cells. The multiple exogenous biologically active molecules can also be introduced into a producer cell using a physical method such as particle-mediated transfection, “gene gun”, biolistics, or particle bombardment technology (Papapetrou et al., Gene Therapy 12: S118-S130 (2005)). A reporter gene such as, for example, beta-galactosidase, chloramphenicol acetyltransferase, luciferase, or green fluorescent protein can be used to assess the transfection efficiency of the producer cell.

In certain aspects, the multiple exogenous biologically active molecules are introduced to the producer cell by viral transduction. A number of viruses can be used as gene transfer vehicles, including moloney murine leukemia virus (MMLV), adenovirus, adeno-associated virus (AAV), herpes simplex virus (HSV), lentiviruses, and spumaviruses. The viral mediated gene transfer vehicles comprise vectors based on DNA viruses, such as adenovirus, adeno-associated virus and herpes virus, as well as retroviral based vectors.

In certain aspects, the multiple exogenous biologically active molecules are introduced to the producer cell by electroporation. Electroporation creates transient pores in the cell membrane, allowing for the introduction of various molecules into the cell. In some aspects, DNA and RNA as well as polypeptides and non-polypeptide therapeutic agents can be introduced into the producer cell by electroporation.

In certain aspects, the multiple exogenous biologically active molecules are introduced to the producer cell by microinjection. In some aspects, a glass micropipette can be used to inject the multiple exogenous biologically active molecules into the producer cell at the microscopic level.

In certain aspects, the multiple exogenous biologically active molecules are introduced to the producer cell by extrusion.

In certain aspects, the multiple exogenous biologically active molecules are introduced to the producer cell by sonication. In some aspects, the producer cell is exposed to high intensity sound waves, causing transient disruption of the cell membrane allowing loading of the multiple exogenous biologically active molecules.

In certain aspects, the multiple exogenous biologically active molecules are introduced to the producer cell by cell fusion. In some aspects, the multiple exogenous biologically active molecules are introduced by electrical cell fusion. In other aspects, polyethylene glycol (PEG) is used to fuse the producer cells. In further aspects, sendai virus is used to fuse the producer cells.

In some aspects, the multiple exogenous biologically active molecules are introduced to the producer cell by hypotonic lysis. In such aspects, the producer cell can be exposed to low ionic strength buffer causing them to burst allowing loading of the one or more moieties. In other aspects, controlled dialysis against a hypotonic solution can be used to swell the producer cell and to create pores in the producer cell membrane. The producer cell is subsequently exposed to conditions that allow resealing of the membrane.

In some aspects, the multiple exogenous biologically active molecules are introduced to the producer cell by detergent treatment. In certain aspects, producer cell is treated with a mild detergent which transiently compromises the producer cell membrane by creating pores allowing loading of the multiple exogenous biologically active molecules. After producer cells are loaded, the detergent is washed away thereby resealing the membrane.

In some aspects, the multiple exogenous biologically active molecules are introduced to the producer cell by receptor mediated endocytosis. In certain aspects, producer cells have a surface receptor which, upon binding of the multiple exogenous biologically active molecules, induces internalization of the receptor and the associated molecules.

In some aspects, the multiple exogenous biologically active molecules are introduced to the producer cell by filtration. In certain aspects, the producer cells and the multiple exogenous biologically active molecules can be forced through a filter of pore size smaller than the producer cell causing transient disruption of the producer cell membrane and allowing the multiple exogenous biologically active molecules to enter the producer cell.

In some aspects, the producer cell is subjected to several freeze thaw cycles, resulting in cell membrane disruption allowing loading of the multiple exogenous biologically active molecules.

### Methods of Modifying an Exosome

In some aspects, a method of producing an exosome comprises modifying the isolated exosome by directly introducing the multiple exogenous biologically active molecules into the EVs. In certain aspects, the multiple exogenous biologically active molecules comprise a therapeutic molecule (e.g., an antigen), adjuvant, immune modulator, targeting moieties, or combinations thereof. In some aspects, an isolated exosome can be further modified by directly introducing a scaffold moiety disclosed herein (e.g., Scaffold X or Scaffold Y) using any of the methods disclosed herein for introducing the multiple exogenous biologically active molecules into the EV, e.g., exosome.

In certain aspects, the multiple exogenous biologically active molecules are introduced to the exosome by transfection. In some aspects, the multiple exogenous biologically active molecules can be introduced into the EV using synthetic macromolecules such as cationic lipids and polymers (Papapetrou et al., Gene Therapy 12: S118-S130 (2005)). In certain aspects, chemicals such as calcium phosphate, cyclodextrin, or polybrene, can be used to introduce the multiple exogenous biologically active molecules to the EV.

In certain aspects, the multiple exogenous biologically active molecules are introduced to the EV by electroporation. In some aspects, exosomes are exposed to an electrical field which causes transient holes in the EV membrane, allowing loading of the multiple exogenous biologically active molecules.

In certain aspects, the multiple exogenous biologically active molecules are introduced to the EV by microinjection. In some aspects, a glass micropipette can be used to inject the multiple exogenous biologically active molecules directly into the EV at the microscopic level.

In certain aspects, the multiple exogenous biologically active molecules are introduced to the EV by extrusion.

In certain aspects, the multiple exogenous biologically active molecules are introduced to the EV by sonication. In some aspects, EVs are exposed to high intensity sound waves, causing transient disruption of the EV membrane allowing loading of the multiple exogenous biologically active molecules.

In some aspects, multiple exogenous biologically active molecules can be conjugated to the surface of the EV. Conjugation can be achieved chemically or enzymatically, by methods known in the art.

In some aspects, the EV comprises multiple (e.g., two or more) exogenous biologically active molecules that are chemically conjugated. Chemical conjugation can be accomplished by covalent bonding of the multiple exogenous biologically active molecules to another molecule, with or without use of a linker. The formation of such conjugates is within the skill of artisans and various techniques are known for accomplishing the conjugation, with the choice of the particular technique being guided by the materials to be conjugated. In certain aspects, polypeptides are conjugated to the EV. In some aspects, non-polypeptides, such as lipids, carbohydrates, nucleic acids, and small molecules, are conjugated to the EV.

In some aspects, the multiple exogenous biologically active molecules are introduced to the EV by hypotonic lysis. In such aspects, the EVs can be exposed to low ionic strength buffer causing them to burst allowing loading of the multiple exogenous biologically active molecules. In other aspects, controlled dialysis against a hypotonic solution can be used to swell the EV and to create pores in the EV membrane. The EV is subsequently exposed to conditions that allow resealing of the membrane.

In some aspects, the multiple exogenous biologically active molecules are introduced to the EV by detergent treatment. In certain aspects, extracellular vesicles are treated with a mild detergent which transiently compromises the EV membrane by creating pores allowing loading of the multiple exogenous biologically active molecules. After EVs are loaded, the detergent is washed away thereby resealing the membrane.

In some aspects, the multiple exogenous biologically active molecules are introduced to the EV by receptor mediated endocytosis. In certain aspects, EVs have a surface receptor which, upon binding of the multiple exogenous biologically active molecules, induces internalization of the receptor and the associated molecules.

In some aspects, the multiple exogenous biologically active molecules are introduced to the EV by mechanical firing. In certain aspects, extracellular vesicles can be bombarded with multiple exogenous biologically active molecules attached to a heavy or charged particle such as gold microcarriers. In some of these aspects, the particle can be mechanically or electrically accelerated such that it traverses the EV membrane.

In some aspects, extracellular vesicles are subjected to several freeze thaw cycles, resulting in EV membrane disruption allowing loading of the multiple exogenous biologically active molecules.

### Methods of Isolating an EV, e.g., Exosome

In some aspects, methods of producing EVs disclosed herein comprises isolating the EV from the producer cells. In certain aspects, the EVs released by the producer cell into the cell culture medium. It is contemplated that all known manners of isolation of EVs are deemed suitable for use herein. For example, physical properties of EVs can be employed to separate them from a medium or other source material, including separation on the basis of electrical charge (e.g., electrophoretic separation), size (e.g., filtration, molecular sieving, etc.), density (e.g., regular or gradient centrifugation), Svedberg constant (e.g., sedimentation with or without external force, etc.). Alternatively, or additionally, isolation can be based on one or more biological properties, and include methods that can employ surface markers (e.g., for precipitation, reversible binding to solid phase, FACS separation, specific ligand binding, non-specific ligand binding, affinity purification etc.).

Isolation and enrichment can be done in a general and non-selective manner, typically including serial centrifugation. Alternatively, isolation and enrichment can be done in a more specific and selective manner, such as using EV or producer cell-specific surface markers. For example, specific surface markers can be used in immunoprecipitation, FACS sorting, affinity purification, and magnetic separation with bead-bound ligands.

In some aspects, size exclusion chromatography can be utilized to isolate the EVs. Size exclusion chromatography techniques are known in the art. Exemplary, non-limiting techniques are provided herein. In some aspects, a void volume fraction is isolated and comprises the EVs of interest. Further, in some aspects, the EVs can be further isolated after chromatographic separation by centrifugation techniques (of one or more chromatography fractions), as is generally known in the art. In some aspects, for example, density gradient centrifugation can be utilized to further isolate the extracellular vesicles. In certain aspects, it can be desirable to further separate the producer cell-derived EVs from EVs of other origin. For example, the producer cell-derived EVs can be separated from non-producer cell-derived EVs by immunosorbent capture using an antigen antibody specific for the producer cell.

In some aspects, the isolation of EVs can involve combinations of methods that include, but are not limited to, differential centrifugation, size-based membrane filtration, immunoprecipitation, FACS sorting, and magnetic separation.

### Methods of Treatment

Present disclosure also provides methods of preventing and/or treating a disease or disorder in a subject in need thereof, comprising administering an EV (e.g., exosome) disclosed herein to the subject. In some aspects, a disease or disorder that can be treated with the present methods comprises a cancer, hemophilia, diabetes, growth factor deficiency, eye diseases, graft-versus-host disease (GvHD), autoimmune diseases, gastrointestinal diseases, cardiovascular diseases, respiratory diseases, allergic diseases, degenerative diseases, infectious diseases, fibrotic diseases, or any combination thereof. In certain aspects, a disease or disorder that can be treated is associated with chronic inflammation. In some aspects, the treatment is prophylactic. In other aspects, the EVs (e.g., exosomes) of the present disclosure are used to induce an immune response. In other aspects, the EVs of the present disclosure are used to vaccinate a subject.

In some aspects, the disease or disorder is a cancer. When administered to a subject with a cancer, in certain aspects, EVs of the present disclosure can up-regulate an immune response and enhance the tumor targeting of the subject's immune system. In some aspects, the cancer being treated is characterized by infiltration of leukocytes (T-cells, B-cells, macrophages, dendritic cells, monocytes) into the tumor microenvironment, or so-called “hot tumors” or “inflammatory tumors”. In some aspects, the cancer being treated is characterized by low levels or undetectable levels of leukocyte infiltration into the tumor microenvironment, or so-called “cold tumors” or “non-inflammatory tumors”. In some aspects, an EV is administered in an amount and for a time sufficient to convert a “cold tumor” into a “hot tumor”, i.e., said administering results in the infiltration of leukocytes (such as T-cells) into the tumor microenvironment. In certain aspects, cancer comprises bladder cancer, cervical cancer, renal cell cancer, testicular cancer, colorectal cancer, lung cancer, head and neck cancer, and ovarian, lymphoma, liver cancer, glioblastoma, melanoma, myeloma, leukemia, pancreatic cancers, or combinations thereof. In other term, “distal tumor” or “distant tumor” refers to a tumor that has spread from the original (or primary) tumor to distant organs or distant tissues, e.g., lymph nodes. In some aspects, the EVs of the disclosure treats a tumor after the metastatic spread.

In some aspects, the disease or disorder is a graft-versus-host disease (GvHD). In some aspects, the disease or disorder that can be treated with the present disclosure is an autoimmune disease. Non-limiting examples of autoimmune diseases include: multiple sclerosis, peripheral neuritis, Sjogren's syndrome, rheumatoid arthritis, alopecia, autoimmune pancreatitis, Behcet's disease, Bullous pemphigoid, Celiac disease, Devic's disease (neuromyelitis optica), Glomerulonephritis, IgA nephropathy, assorted vasculitides, scleroderma, diabetes, arteritis, vitiligo, ulcerative colitis, irritable bowel syndrome, psoriasis, uveitis, systemic lupus erythematosus, and combinations thereof.

In some aspects, the disease or disorder is an infectious disease. In certain aspects, the disease or disorder is an oncogenic virus. In some aspects, infectious diseases that can be treated with the present disclosure includes, but not limited to, Human Gamma herpes virus 4 (Epstein Barr virus), influenza A virus, influenza B virus, cytomegalovirus, Staphylococcus aureus, Mycobacterium tuberculosis, Chlamydia trachomatis, HIV-1, HIV-2, corona viruses (e.g., MERS-CoV and SARS CoV), filoviruses (e.g., Marburg and Ebola), Streptococcus pyogenes, Streptococcus pneumoniae, Plasmodia species (e.g., vivax and falciparum), Chikungunya virus, Human Papilloma virus (HPV), Hepatitis B, Hepatitis C, human herpes virus 8, herpes simplex virus 2 (HSV2), Klebsiella sp., Pseudomonas aeruginosa, Enterococcus sp., Proteus sp., Enterobacter sp., Actinobacter sp., coagulase-negative staphylococci (CoNS), Mycoplasma sp., or combinations thereof.

In some aspects, the EVs (e.g., exosomes) are administered intravenously to the circulatory system of the subject. In some aspects, the EVs are infused in suitable liquid and administered into a vein of the subject.

In some aspects, the EVs (e.g., exosomes) are administered intra-arterially to the circulatory system of the subject. In some aspects, the EVs are infused in suitable liquid and administered into an artery of the subject.

In some aspects, the EVs (e.g., exosomes) are administered to the subject by intrathecal administration. In some aspects, the EVs are administered via an injection into the spinal canal, or into the subarachnoid space so that it reaches the cerebrospinal fluid (CSF).

In some aspects, the EVs (e.g., exosomes) are administered intratumorally into one or more tumors of the subject.

In some aspects, the EVs (e.g., exosomes) are administered to the subject by intranasal administration. In some aspects, the EVs can be insufflated through the nose in a form of either topical administration or systemic administration. In certain aspects, the EVs are administered as nasal spray.

In some aspects, the EVs (e.g., exosomes) are administered to the subject by intraperitoneal administration. In some aspects, the EVs are infused in suitable liquid and injected into the peritoneum of the subject. In some aspects, the intraperitoneal administration results in distribution of the EVs to the lymphatics. In some aspects, the intraperitoneal administration results in distribution of the EVs to the thymus, spleen, and/or bone marrow. In some aspects, the intraperitoneal administration results in distribution of the EVs to one or more lymph nodes. In some aspects, the intraperitoneal administration results in distribution of the EVs to one or more of the cervical lymph node, the inguinal lymph node, the mediastinal lymph node, or the sternal lymph node. In some aspects, the intraperitoneal administration results in distribution of the EVs to the pancreas.

In some aspects, the EVs, e.g., exosomes, are administered to the subject by periocular administration. In some aspects, the s are injected into the periocular tissues. Periocular drug administration includes the routes of subconjunctival, anterior sub-Tenon's, posterior sub-Tenon's, and retrobulbar administration.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1: Characterization of Anti-Clec9A EV (e.g., Exosome) Constructs

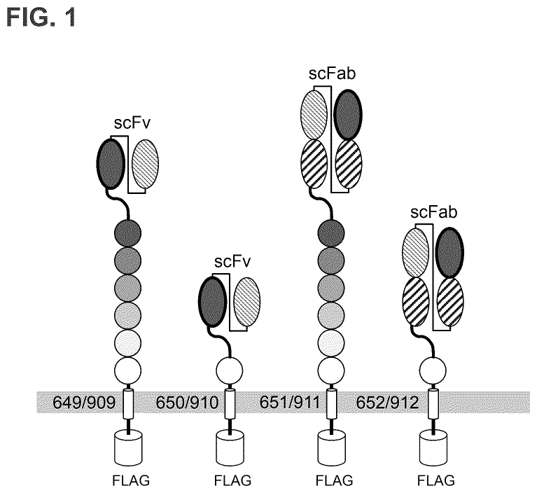

To assess the efficacy of using targeting moieties to increase the uptake of EVs (e.g., exosomes) by specific cells of interest, four different anti-Clec9a exosome constructs were generated. FIG. 1 provides a schematic of the different targeting moieties that were expressed on the exosome constructs: (i) anti-Clec9a single-chain Fv fragment linked to a full-length Scaffold X protein (“scFab-FLPrX”); (ii) anti-Clec9a single-chain Fv fragment linked to a truncated Scaffold X protein (“ScFv-SLPrX”); (iii) anti-Clec9a single-chain F(ab) fragment linked to a full-length Scaffold X protein (“scFab-FLPrX”); and (iv) anti-Clec9a single-chain F(ab) fragment linked to a truncated Scaffold X protein (“scFab-SLPrX”). Briefly, the sequence of the anti-mouse Clec9A clone 10B4 antibody (see Caminschi, et al., Blood 112:3264 (2008); U.S. Publication No. 2013/0273150) was used to design the single-chain F(ab) or Fv antibody fragments. The antibody fragments were expressed on the exterior surface of the exosomes using either truncated or full-length Scaffold X protein. Each of the exosomes also contained a green fluorescent protein (GFP) tag to assist in expression analysis.

To begin characterizing the constructs, exosomes expressing the different anti-Clec9a targeting moieties were used to transfect HEK293 cells. Then, the percentage of transfected cells that were GFP⁺ was determined by flow cytometry. GFP expression in the exosomes produced by the transfected HEK293 cells was also assessed to determine the relative amount of the targeting moieties expressed by the exosomes. Additionally, the binding of the different exosomes to both mouse and human Clec9a protein was assessed using OCTET analysis.

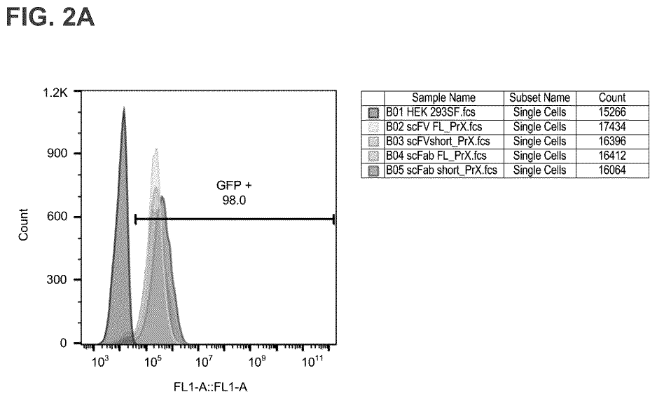

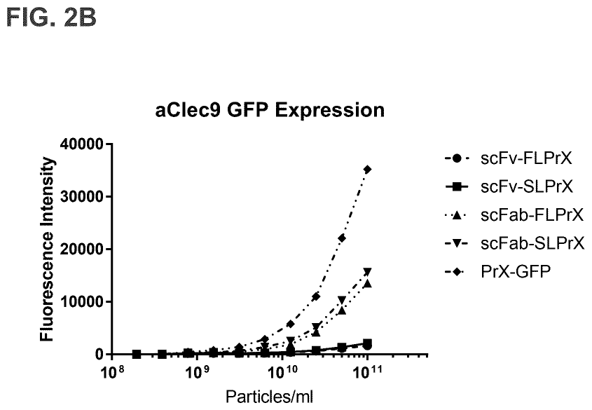

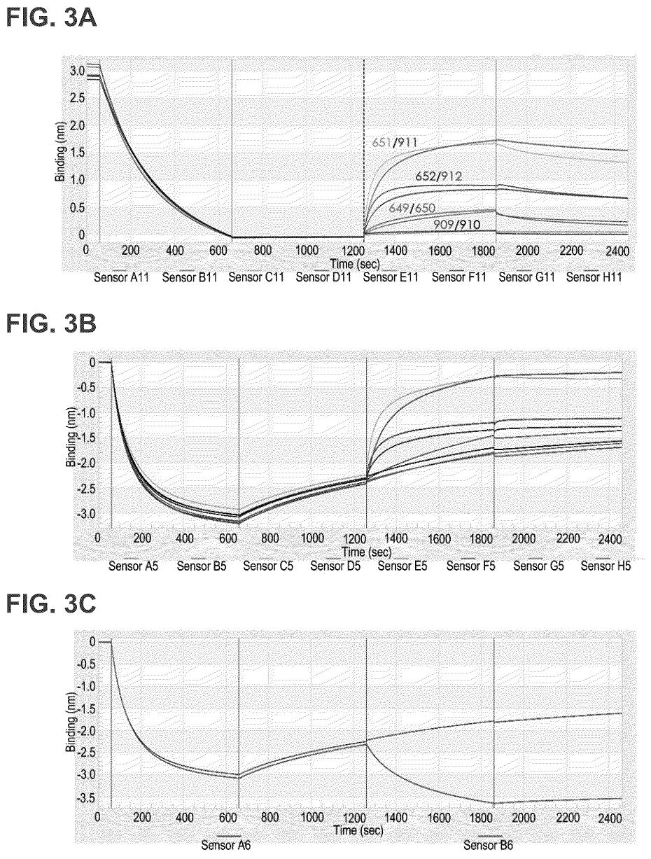

As shown in FIG. 2A, the GFP expression was comparable among the HEK293 cells transfected with the different anti-Clec9a targeting moiety constructs, suggesting that the different constructs had comparable transfection efficiency. However, noticeable differences were observed regarding the amount of targeting moiety expressed on the exosomes and the ability of the exosomes to bind to mouse and human Clec9a protein. As shown in FIG. 2B, among the different constructs tested, anti-Clec9a single-chain F(ab) antibody fragment linked to either the full-length or the truncated Scaffold X protein were most expressed by the exosomes. Regarding their binding ability, as shown in FIGS. 3A-3C, the exosome construct expressing anti-Clec9a single-chain F(ab) linked to a full-length scaffold X protein (i.e., anti-Clec9a scFab-FLPrX) exhibited the greatest binding to both mouse and human Clec9A protein. Therefore, this exosome construct was selected for further analysis.

### Example 2: Analysis of Uptake in Isolated Human Dendritic Cells

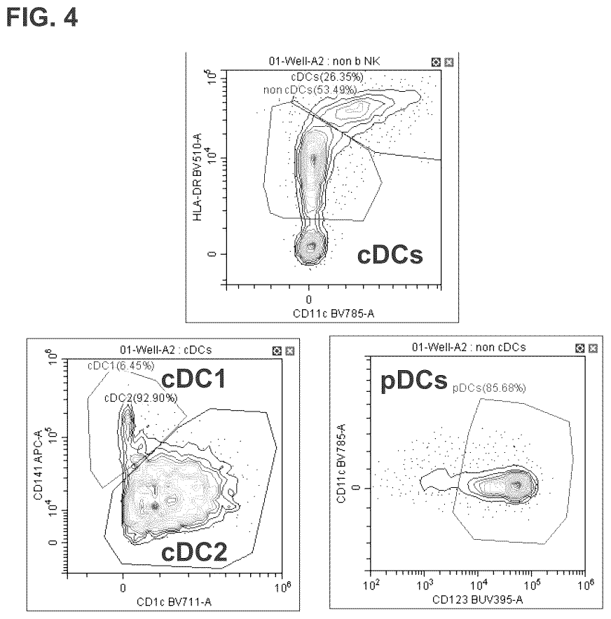

To assess whether the anti-Clec9a scFab-FLPrX exosome construct described in Example 1 can specifically target cells expressing human Clec9a protein, pan-dendritic cell negative bead-based RoboSep kit was used to purify dendritic cells (DCs) from four different human donor PBMCs. The purified DCs were incubated overnight with different concentrations of the exosome constructs. As a control, some of the DCs were incubated with exosomes expressing Scaffold X protein alone. Then, the uptake of the exosomes by different populations of DCs (i.e., plasmacytoid DC (pDC), conventional DC 1 (cDC1), and conventional DC 2 (cDC2)) was measured using flow cytometry. The gating strategy for the different DC subsets are provided in FIG. 4.

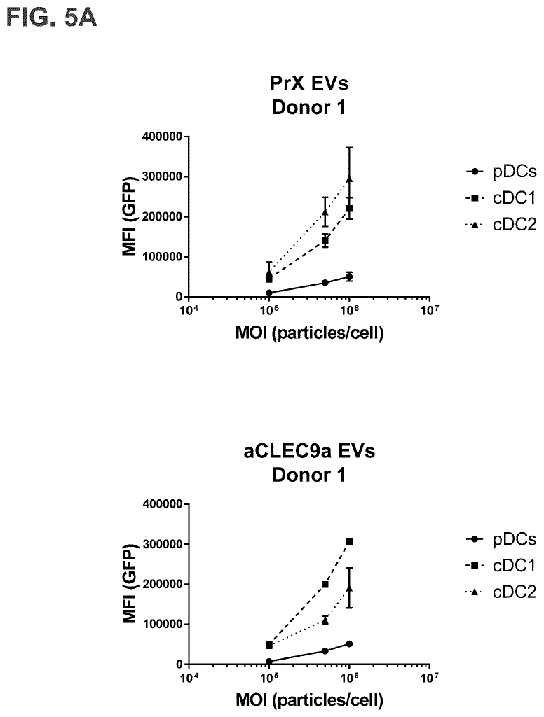

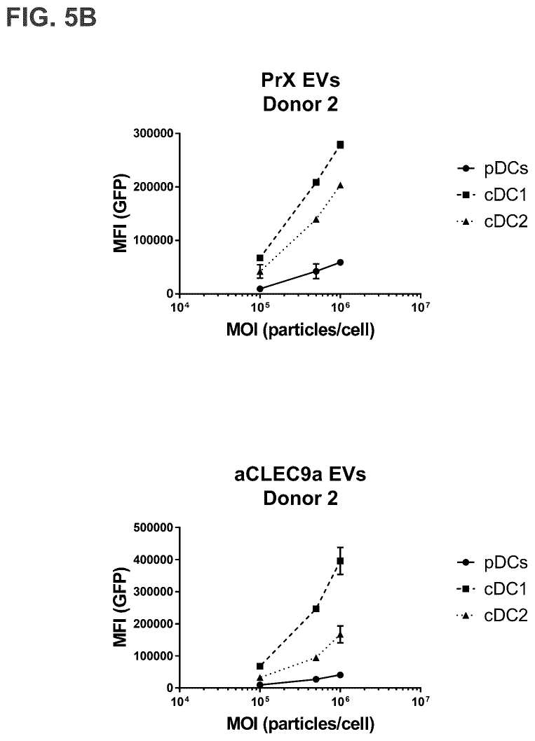

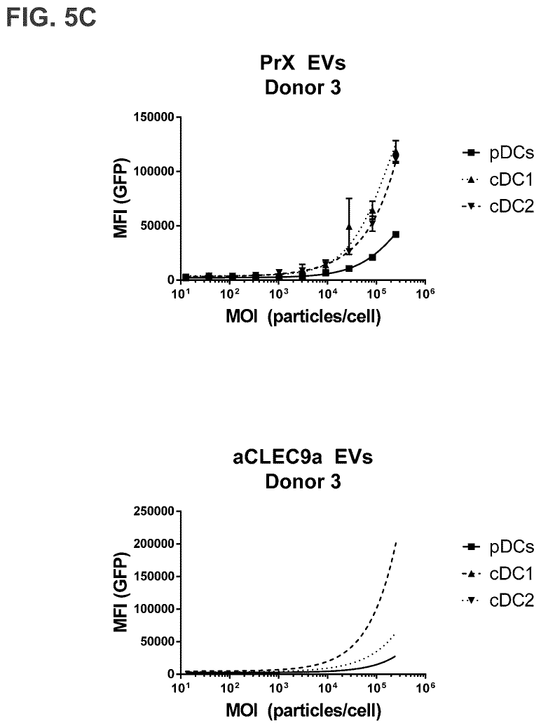

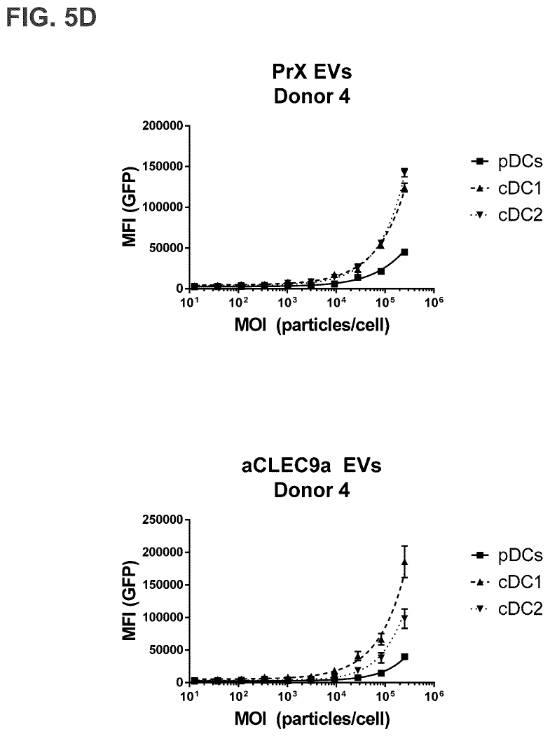

As shown in FIGS. 5A-5D (bottom graph), there was a preferential uptake of the anti-Clec9a scFab-FLPrX EV construct by cDC1 cells among all four donors. In contrast, the uptake of EVs expressing Scaffold X alone varied among the different donors. FIGS. 5A-5D (top graph). For instance, in Donor 1, the greatest uptake of the control EV (i.e., expressing Scaffold X alone) was observed among the cDC2 population. However, in Donor 2, the greatest uptake was observed among cDC1 cells.

The above results demonstrate that the anti-Clec9a scFab-FLPrX exosome construct can specifically target human cDC1 cells, resulting in greater uptake of the exosome by the cDC1 cells.

### Example 3: Analysis of Uptake in Isolated Mouse Dendritic Cells

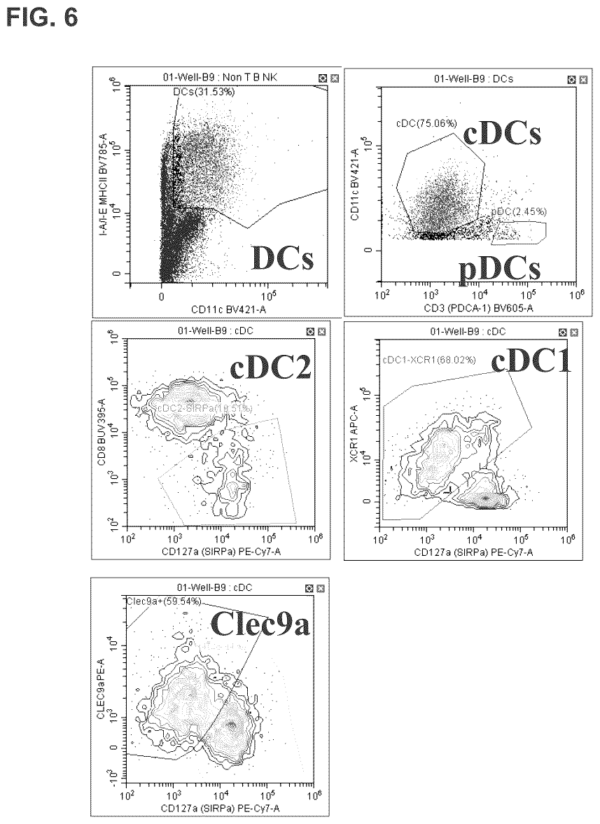

To assess the ability to preferentially target cells expressing mouse Clec9A protein, total dendritic cells were purified from mouse splenocytes. The cells were then cultured overnight with different concentrations of either anti-Clec9a scFab-FLPrX exosome or the control exosome construct (i.e., expressing Scaffold X protein alone). The uptake of the exosome constructs by different DC populations was again assessed using flow cytometry. The different DC populations were identified using the following phenotypic markers: (i) pDCs: CD317⁺, XCR1⁻, Sirpα⁻; (ii) cDC1:, XCR1⁺, Clec9a⁺, Sirpα⁻; and (iii) cDC2:, CD8⁺, CD11b⁺, Sirpα⁺, XCR1⁻, CD1c,b⁺. FIG. 6.

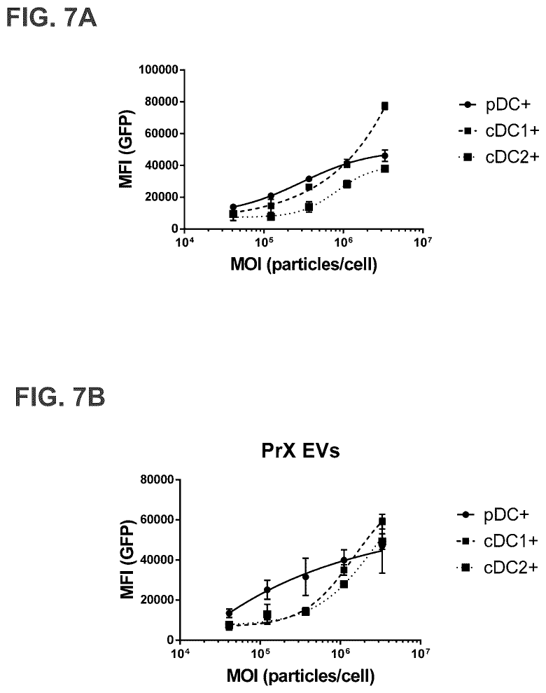

As observed earlier with the human DCs, there was a preferential uptake of the anti-Clec9a scFab-FLPrX exosome construct by the cDC1 population compared to the other DC populations. FIG. 7A. In contrast, the uptake of the control exosome (i.e., expressing Scaffold X protein alone) was comparable among the different DC populations. FIG. 7B.

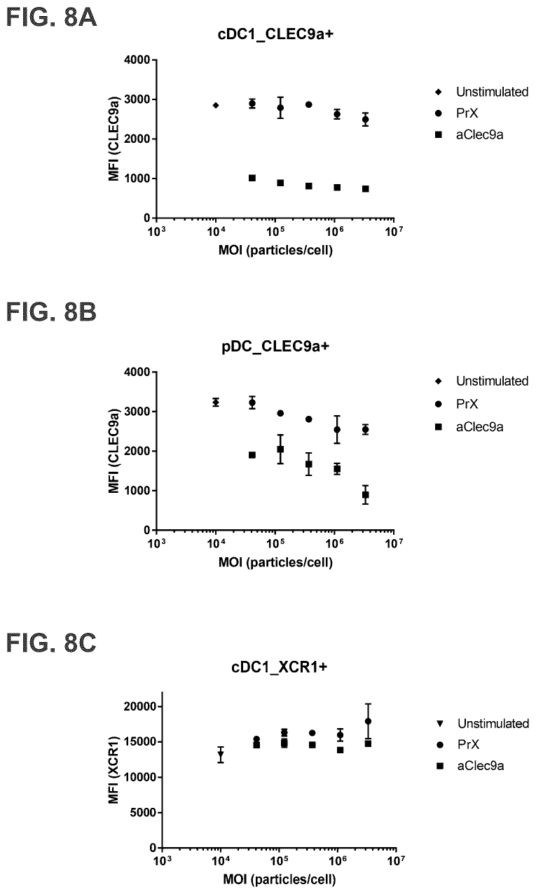

To determine whether the increased uptake was due to Clec9a binding, the expression of Clec9a protein was assessed in the different DC populations after culturing the cells with the exosome constructs. As shown in FIGS. 8A and 8B, there was no significant downregulation of Clec9a expression among both cDC1 and pDC cells when treated with the control EV. However, with the anti-Clec9a scFab-FLPrX exosome construct, significant downregulation of Clec9a expression was observed among cDC1 cells and pDCs. Downregulation was specific to Clec9a protein, as no significant difference was observed in the expression of other non-Clec9a proteins (e.g., XCR1) among the cDC1 cells for both exosome constructs. FIG. 8C.

The above results demonstrate that the anti-Clec9a scFab-FLPrX exosome construct can also specifically target mouse cDC1 cells.

### Example 4: Analysis of Uptake in Clec9a-Expressing HEK Cells

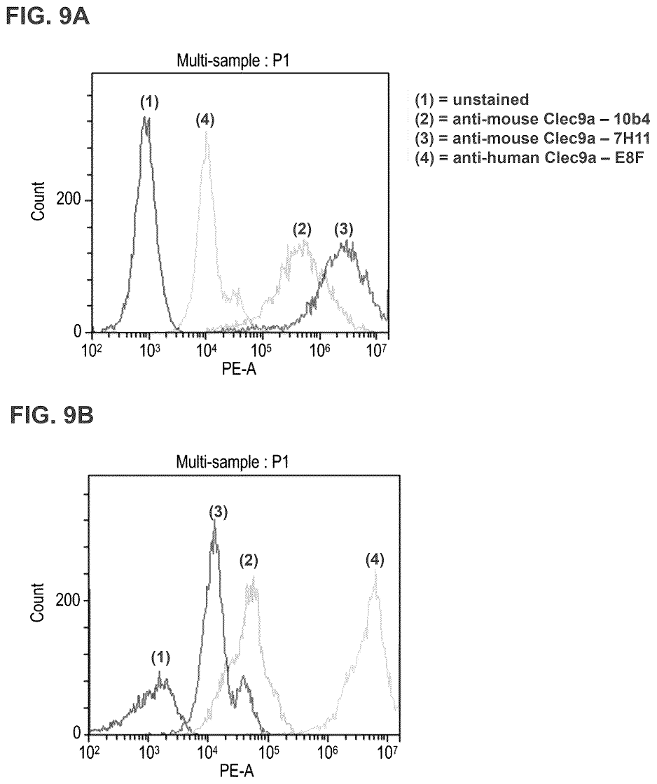

To further confirm the ability of anti-Clec9a scFab-FLPrX exosomes to specifically target Clec9a-expressing cells, human embryonic kidney (HEK) cells were transfected with either human or mouse Clec9a protein. Then, the expression of Clec9a by the transfected cells was confirmed using three different anti-Clec9a antibodies: (i) anti-mouse 10b4 antibody; (ii) anti-mouse 7h11 antibody; and (iii) anti-human E8F antibody. As shown in FIGS. 9A and 9B, both mouse and human Clec9a was stably expressed in the HEK cells and were detectable by the different anti-Clec9a antibodies.

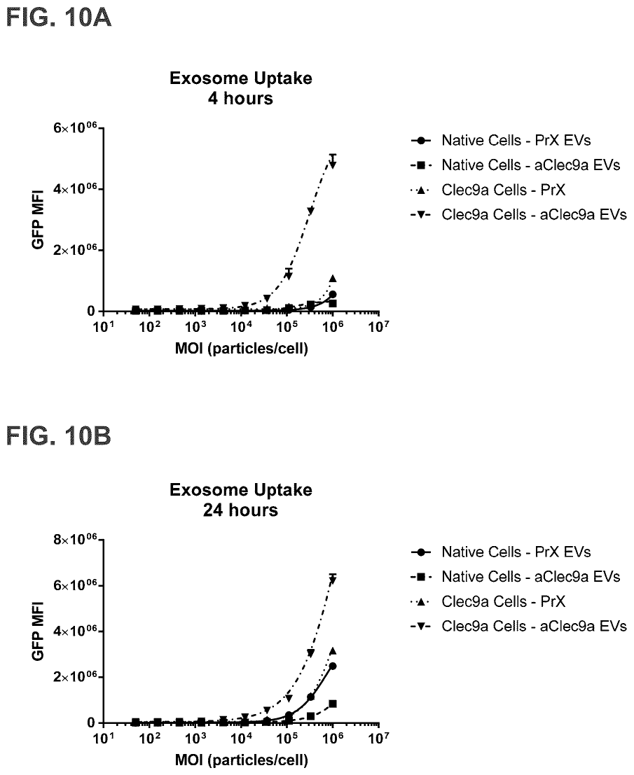

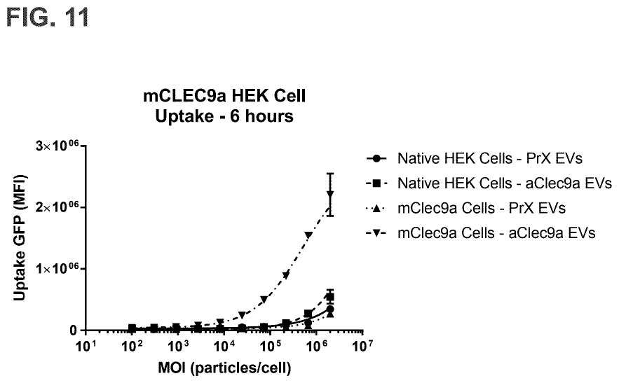

Next, the Clec9a-expressing HEK cells were cultured with either the anti-Clec9a scFab-FLPrX exosome construct or the control exosome (i.e., expressing Scaffold X protein alone). The exosomes further comprised GFP protein tag, which was used to measure exosome uptake in the HEK cells. As shown in FIGS. 10A and 10B, significant uptake of anti-Clec9a scFab-FLPrX EVs was observed among human Clec9a-expressing HEK cells. The uptake was specific to Clec9a expression, as there was minimal uptake observed among the non-transfected HEK cells (i.e., do not express Clec9a). Similarly, there was no significant uptake of the control EV among both the transfected and non-transfected HEK cells. The increase in uptake by human Clec9a-expressing HEK cells was apparent as early as 4 hours post incubation. FIG. 10A. Similar results were observed with mouse Clec9a-expressing HEK cells. FIG. 11.

Collectively, the above results confirm the specificity of the anti-Clec9a targeting moieties disclosed herein, and suggest that EVs expressing such targeting moieties can specifically target Clec9a expressing cells, such as cDC1 cells.

### Example 5: Evaluation of Immune Cell Biodistribution of Anti-Clec9a-Expressing EVs (e.g., Exosomes) In Vivo

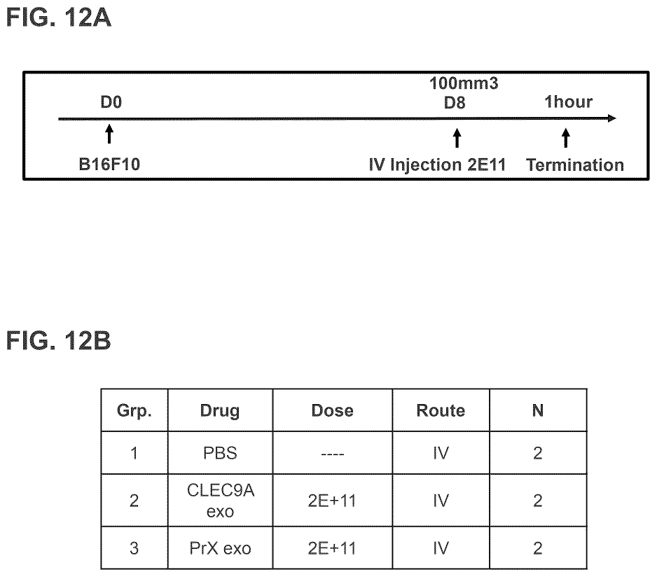

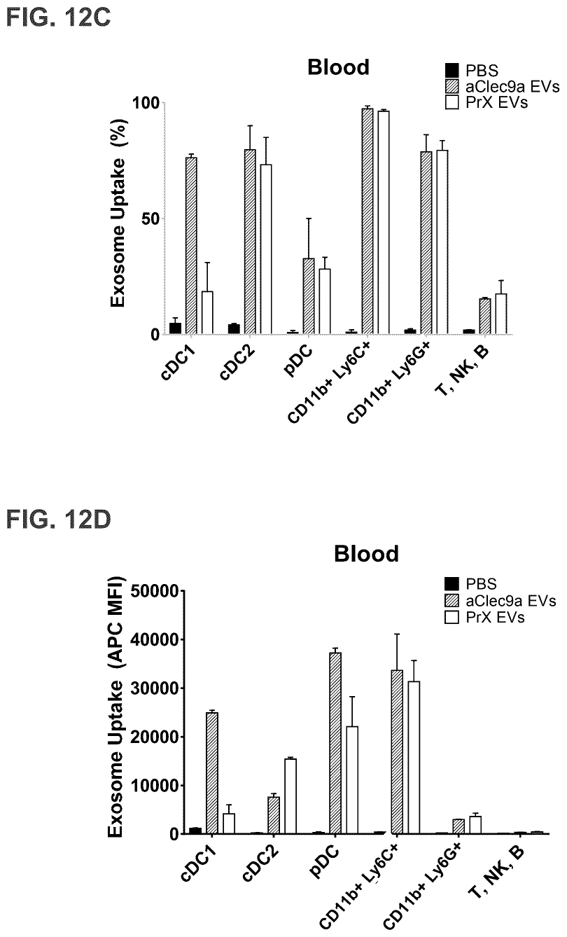

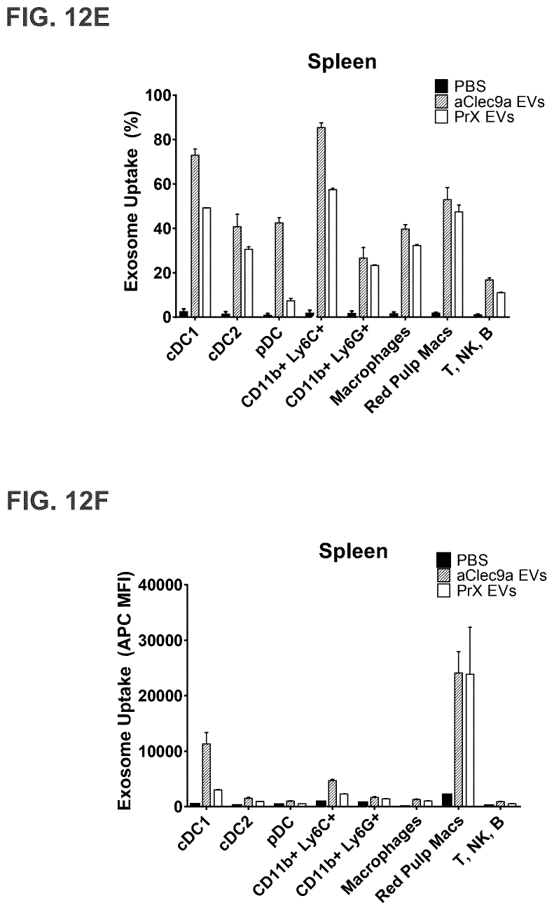

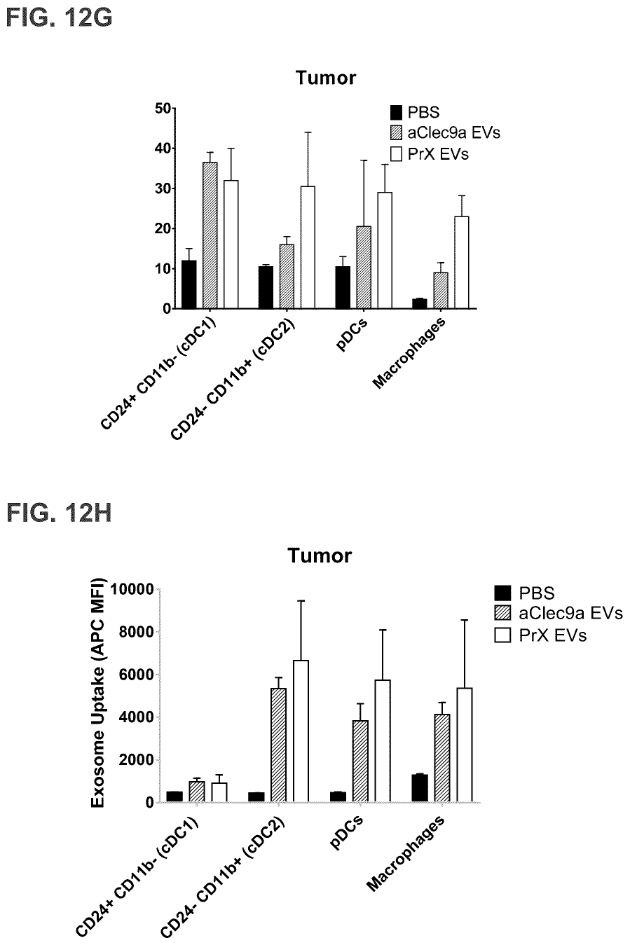

To assess whether EVs (e.g., exosomes) expressing anti-Clec9a targeting moiety can specifically target Clec9a-expressing cells in vivo, B16F10 tumor (melanoma) mouse model was used. As shown in FIG. 12A, the mice were inoculated with B16 tumor cells. When the average size of the tumors had reached 100 mm³ (approximately day 8 post tumor induction), the animals received a single intravenous administration of one of the following: (i) PBS alone; (ii) anti-Clec9a-expressing exosome; or (iii) control exosome (i.e., expressing Scaffold X protein alone). FIG. 12B provides the different treatment groups. The exosomes were administered to the animals at a dose of 2×10¹¹ particles. The animals were sacrificed 1 hour after exosome administration and exosome uptake was assessed in the blood, spleen, and tumor. All the exosomes were labeled with Cy5-ASOs and therefore, uptake was assessed by measuring for Cy5-ASO expression using flow cytometry.

As shown in FIG. 12C, within the blood, there was no noticeable difference in exosome uptake among animals treated with either of the EVs (i.e., Groups II and III in FIG. 12B) for the following cell types: (i) cDC2, (ii) pDC, (iii) CD11b+ Ly6C+ (monocytes); (iv) CD11b+ Ly6G+ (neutrophils); and (v) T, NK, B cells. However, there was a significant difference in uptake among the cDC1 population. As shown in FIG. 12C, greater percentage of cDC1 cells in the blood of animals treated with the anti-Clec9a-expressing exosome were positive for Cy5-ASO expression compared to the control animals (i.e., Group III in FIG. 12B). The amount of the exosome taken up by the cDC1 (as measured by the MFI of Cy5-ASO expression) was also greater among animals treated with the anti-Clec9a-expressing exosomes compared to the control animals. FIG. 12D. Similar results were observed for pDC cells, which are also thought to express Clec9a protein.

Interestingly, within the spleen, there appeared to be a modest to significant increase in exosome uptake among other populations of cells (other than cDC1) in animals treated with the anti-Clec9a-expressing exosome. FIG. 12E. However, when the amount of exosomes that were taken up by the cells was assessed, there was only a significant difference within the cDC1 cells. FIG. 12F. This result suggests that while some of the cells within the spleen can preferentially uptake exosomes expressing an anti-Clec9a targeting moiety, such exosomes, nonetheless, exhibit tropism towards cDC1, which are known to express Clec9a protein.

Within the tumor, the difference in uptake was not as apparent as observed in the blood and in the spleen. However, a modest increase in exosome uptake was observed for the cDC1 population in animals treated with the anti-Clec9a-expressing exosomes compared to the control animals. FIGS. 12G and 12H.

The above results confirm the earlier in vitro data and demonstrates that EVs (e.g., exosomes) expressing anti-Clec9a targeting moieties can preferentially target Clec9a-expressing cells, such as cDC1 and pDC cells.

### Example 6: Analysis of the Effect of Route of Administration on Immune Cell Biodistribution of Anti-Clec9a EVs (e.g., Exosomes)

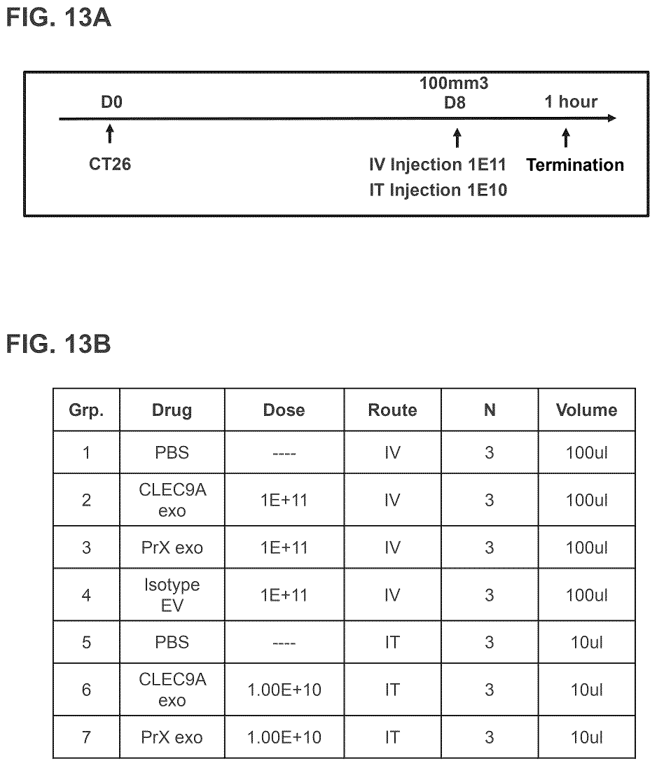

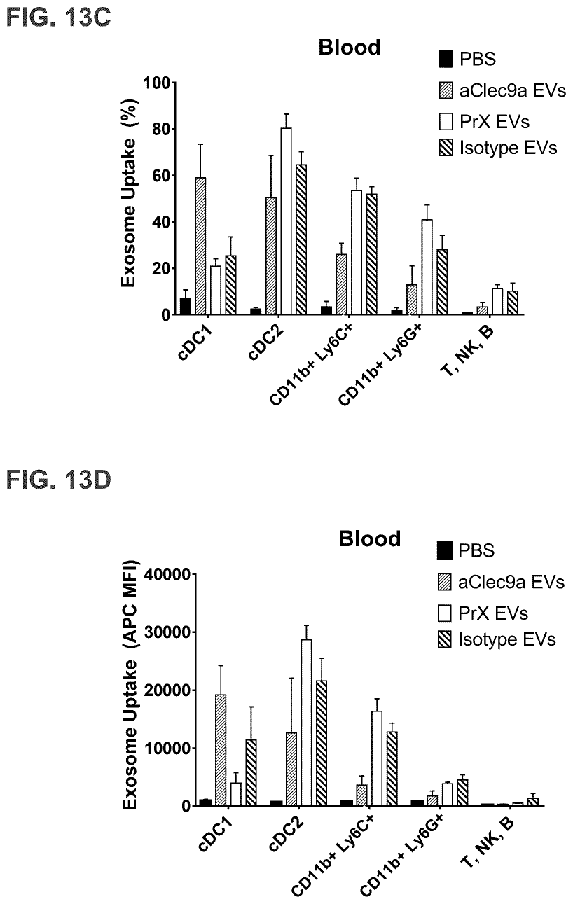

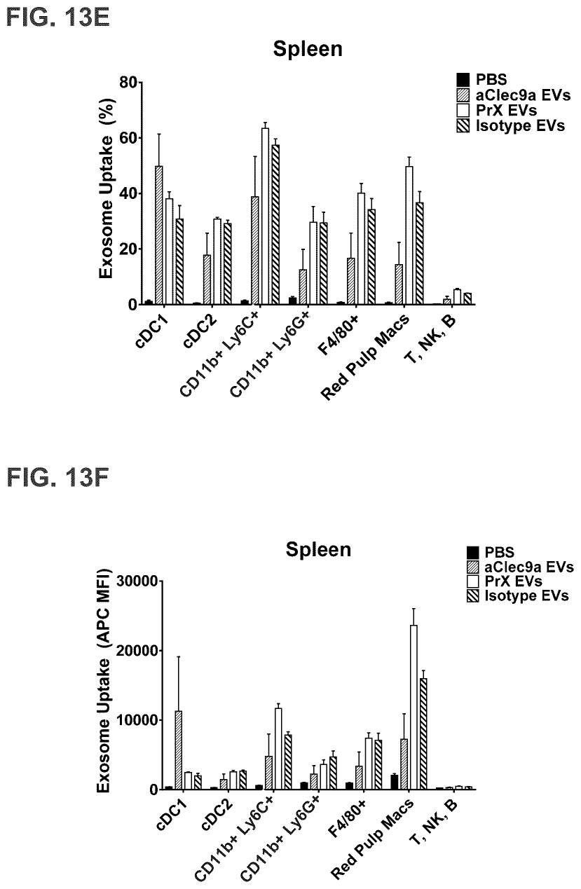

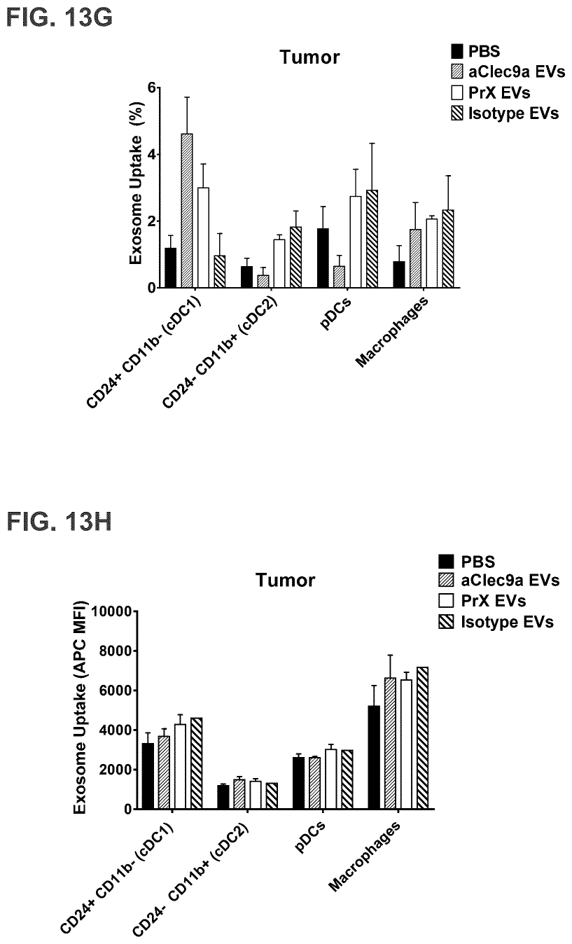

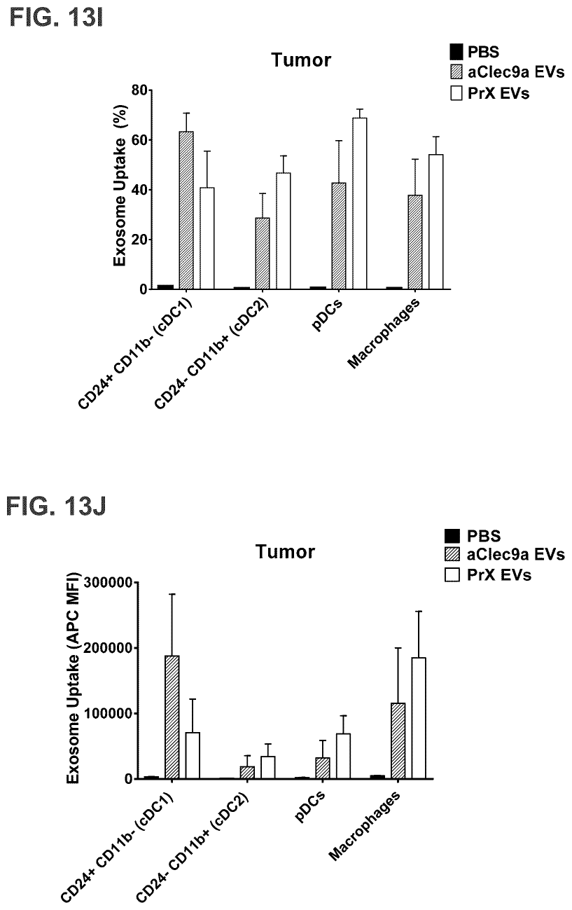

To confirm the results from the B16F10 tumor model and also to determine whether the route of administration can affect immune cell biodistribution of anti-Clec9a exosomes, CT26 tumor mouse model was used. As shown in FIG. 13A, mice were inoculated with CT26 tumor cells. When the size of the tumor reached 100 mm³ (approximately day 8 post tumor induction), the animals received a single administration (either intravenously or intratumorally) of one of the following: (i) PBS alone; (ii) anti-Clec9a-expressing exosome (“aClec9a EVs”); (iii) exosome expressing Scaffold X protein alone (“PrX EVs”); or (iv) exosome expressing a non-relevant antibody (“Isotype EVs”). For the intravenous administration, the exosomes were administered at a dose of 1×10¹¹ particles. For the intraturmoral administration, the exosomes were administered at a dose of 1×10¹⁰ particles. The animals were then sacrificed approximately 1 hour later, and the uptake of the exosomes was assessed in the blood, spleen, and/or tumor. FIG. 13B provides the different treatment groups.

As observed in the B16F10 tumor mouse model (see Example 6), cDC1 cells from both the blood (FIGS. 13C and 13D) and the spleen (FIGS. 13E and 13F) exhibited greater uptake of the anti-Clec9a exosomes compared to the other exosomes. With the other cell types analyzed (i.e., cDC2, CD11b+ Ly6C+ (monocytes), CD11b+ Ly6G+ (neutrophils), T, NK, and B cells), there was no preferential uptake of the anti-Clec9a exosomes. In fact, these other cell types appeared to exhibit greater uptake of the control exosomes (i.e., expressing Scaffold X protein alone or expressing a non-relevant antibody) compared to the anti-Clec9a exosomes. The amount of the exosome taken up by the cDC1 (as measured by the MFI of Cy5-ASO expression) was also consistent with that observed earlier in the B16F10 tumor mouse model. Greater amount of the exosomes were taken up by the cDC1 cells in animals treated with the anti-Clec9a exosome, compared to the control animals.

As shown in FIGS. 13I-13J, similar results were observed in the tumors. Regardless of the route of administration (i.e., intravenous (FIGS. 13G and 13H); intratumoral (FIGS. 131 and 13J)), in animals that received the anti-Clec9a exosomes, there was a preferential uptake of the exosome by the DC1 cells within the tumor.

These results confirm that EVs (e.g., exosomes) expressing anti-Clec9a targeting moieties can preferentially target Clec9a-expressing cells in vivo. These results further suggest that the increased tropism of anti-Clec9a exosomes to Clec9a-expressing cells is not dependent on the route of administration.

### Example 7: Analysis of Uptake of EVs (e.g., Exosomes) Expressing Anti-Clec9a Targeting Moiety and a STING Agonist

The ability to direct STING agonist to specific immune cells can help decrease toxicity and/or increase potency of the EVs (e.g., exosomes). Therefore, anti-Clec9a exosomes described in the earlier examples were further loaded with a STING agonist. Briefly, 1 mM STING agonist including ML RR-S2 CDA ammonium salt (MedChem Express, Cat. No. HY-12885B) and (3-3 cAIMPdFSH; InvivoGen, Cat. No. tlrl-nacairs) was incubated with purified exosomes (1E12 total particles) in 300 ul of PBS at 37° C. overnight. The mixture was then washed twice in PBS and purified by ultra-centrifugation at 100,000×g. Then, the ability of the exosomes to target the STING pathway in STING-reporter HEK cells transfected with either human or mouse Clec9a protein was assessed.

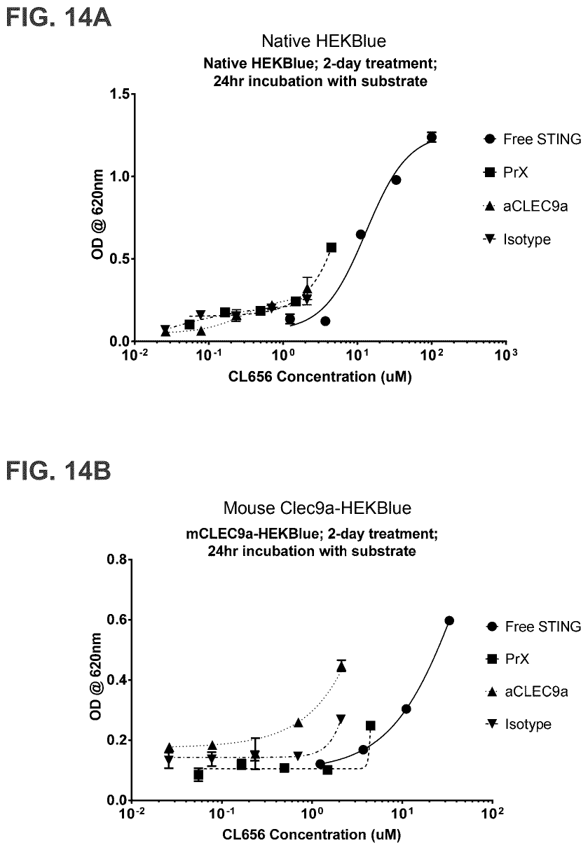

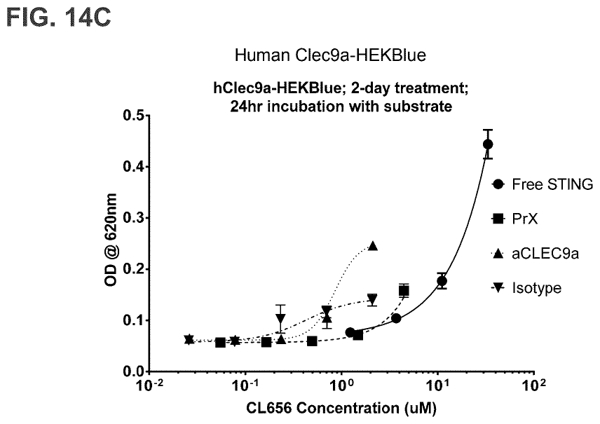

As shown in FIG. 14A, there was no significant difference in STING activity in non-transfected reporter HEK cells (i.e., do not express Clec9a protein). However, for reporter HEK cells transfected with either mouse or human Clec9 protein, a greater activation of STING pathway was observed, when the cells were treated with the anti-Clec9a exosome compared to free STING agonist (i.e., not loaded in an exosome). FIGS. 14B and 14C. This result is in agreement with the earlier examples and demonstrates that anti-Clec9a EVs (e.g., exosomes) expressing STING agonist can be used to preferentially deliver STING agonist to Clec9a protein expressing cells, e.g., cDC1.

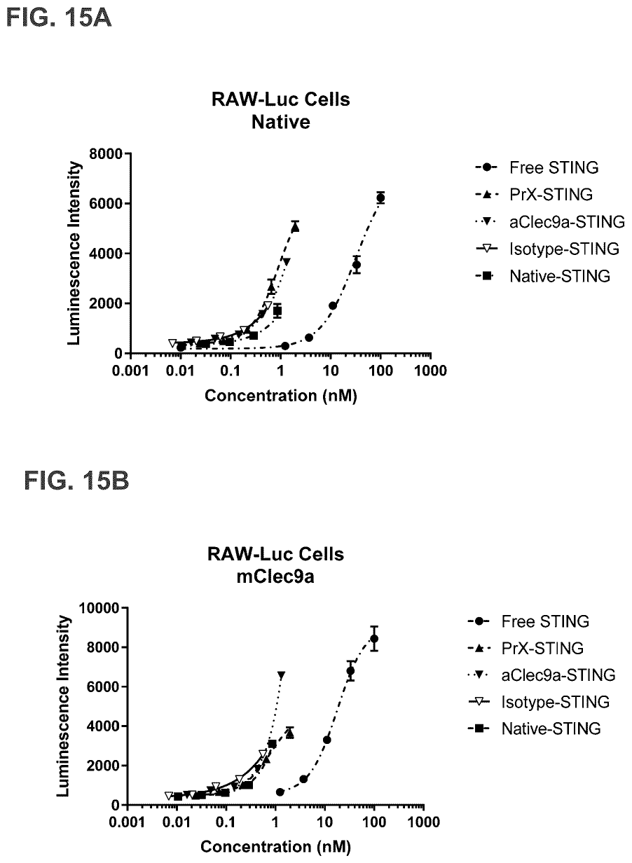

To confirm the above results, the uptake of the anti-Clec9a exosomes loaded with STING agonist was also assessed in STING-reporter Raw-luciferase cells that have been modified to express mouse Clec9a protein. As shown in FIGS. 15A and 15B, similar results were observed. In native STING-reporter Raw-luciferase cells (i.e., do not express Clec9a protein), no significant differences in STING activity was observed among the different groups. However, in STING-reporter Raw-luciferase cells expressing Clec9a, significantly greater STING activity was observed when the cells were treated with the anti-Clec9a exosomes loaded with STING agonist.

The above results confirm that the expression of anti-Clec9a can selectively target the EVs (e.g., exosomes) to Clec9a expressing cells, such as dendritic cells.

### Example 8: Analysis of Cytokine Production after Treatment with Anti-Clec9a EVs (e.g., Exosomes)

To further assess the ability of anti-Clec9a EVs (e.g., exosomes) to specifically target Clec9a expressing cells, STING activity was assessed in both total mouse splenocytes and isolated mouse splenic dendritic cells (DCs) after stimulation with anti-Clec9a exosomes loaded with STING agonist. STING activity was measured as cytokine production using a multiplex panel consisting of thirteen different cytokines, i.e., IFNβ, CXCL10, CCL5, IL-6, IL-12, IFNα, TNFα, GMC-SF, IFNγ, IL-10, CCL2, IL-1β, and CXCL1.

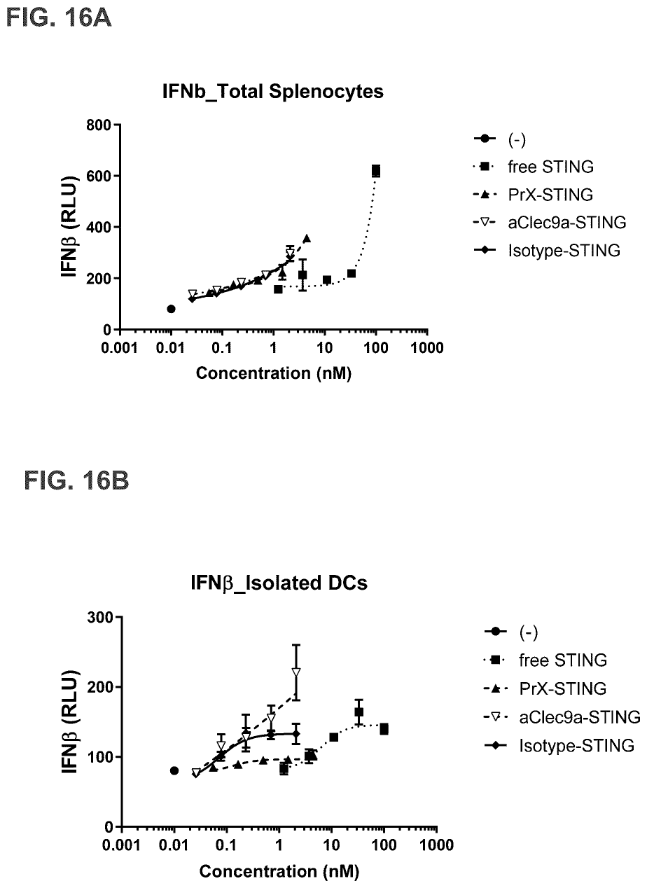

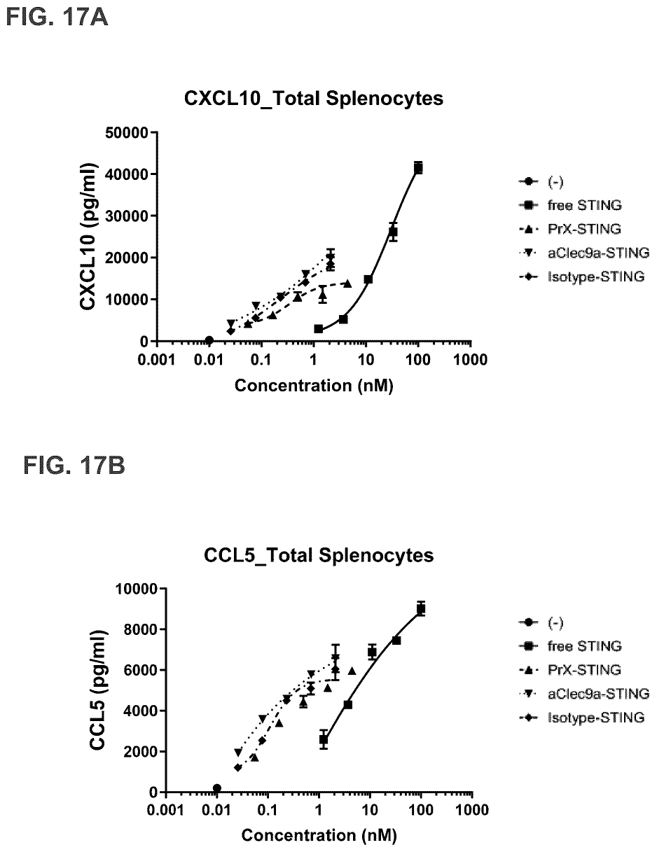

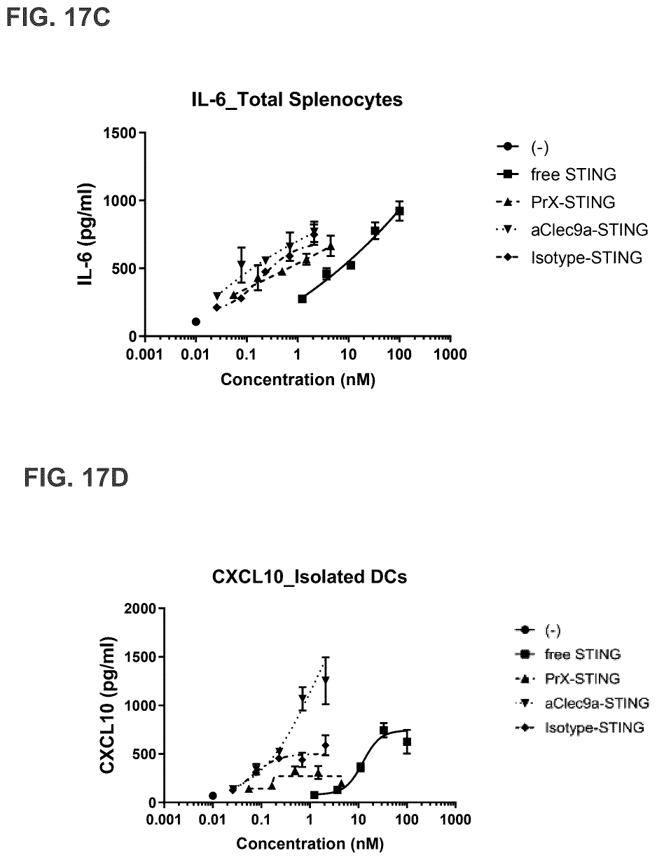

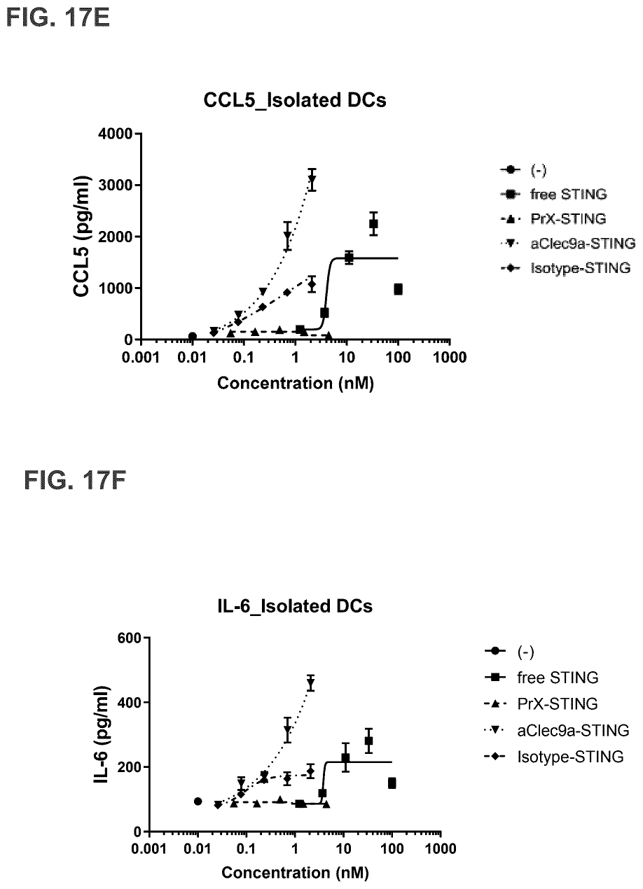

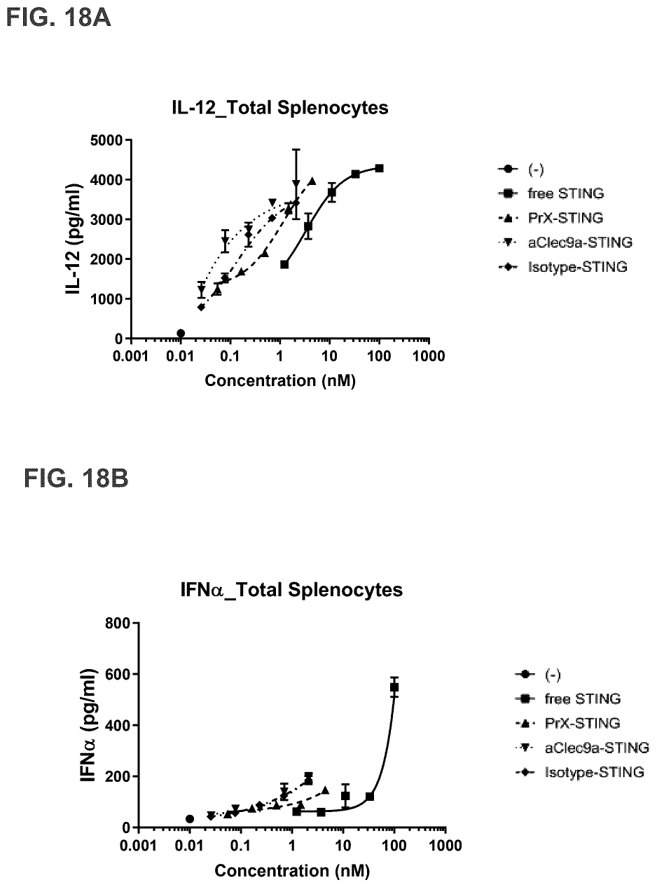

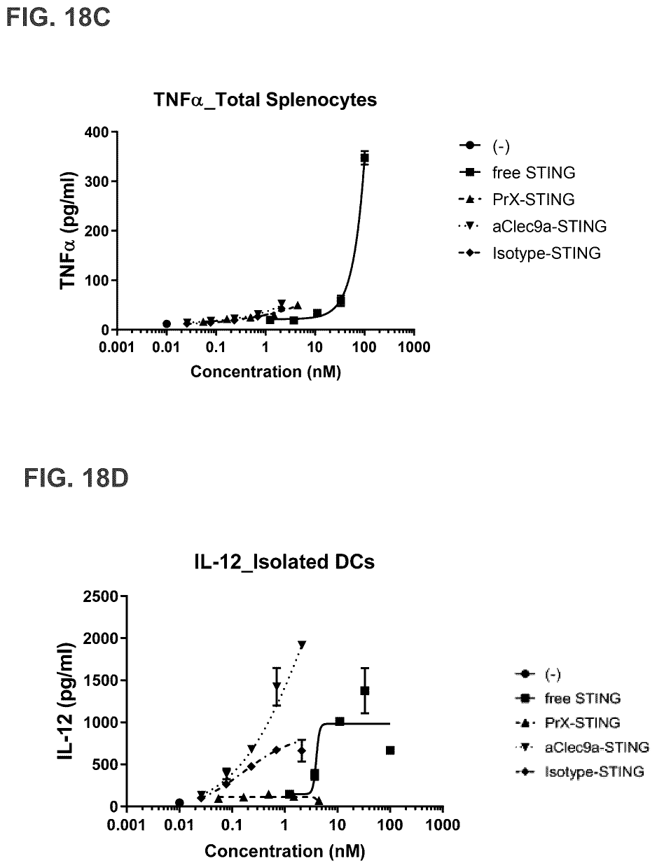

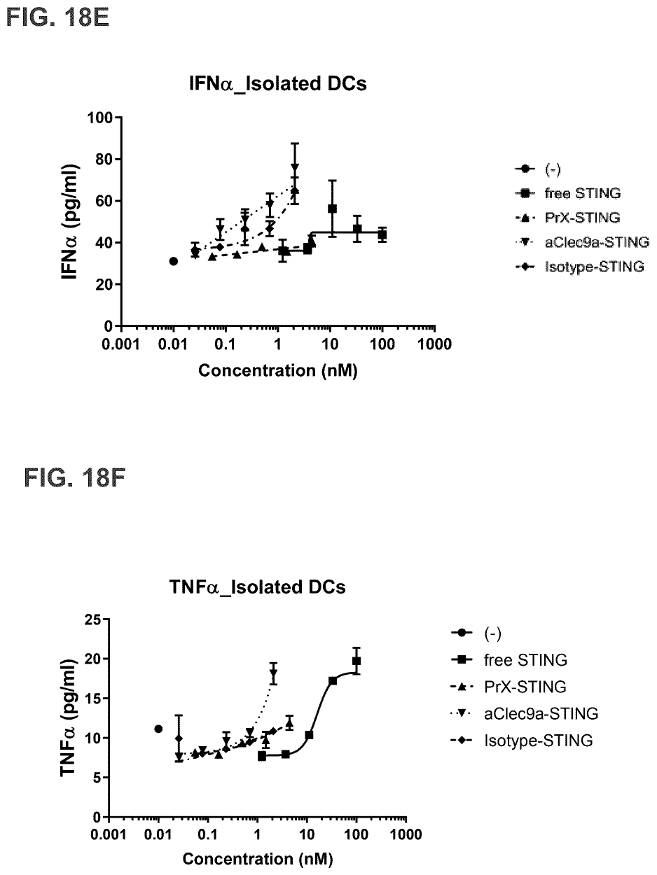

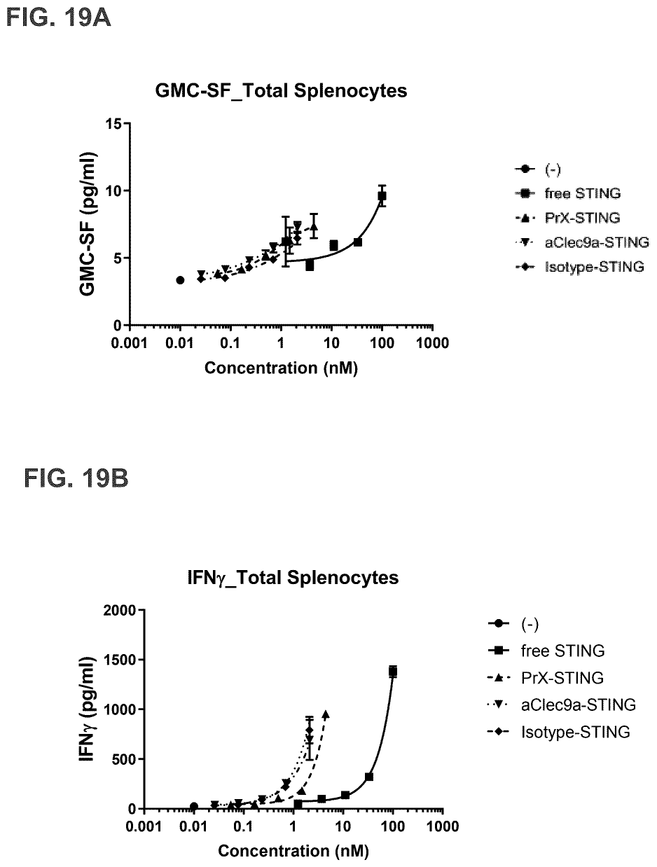

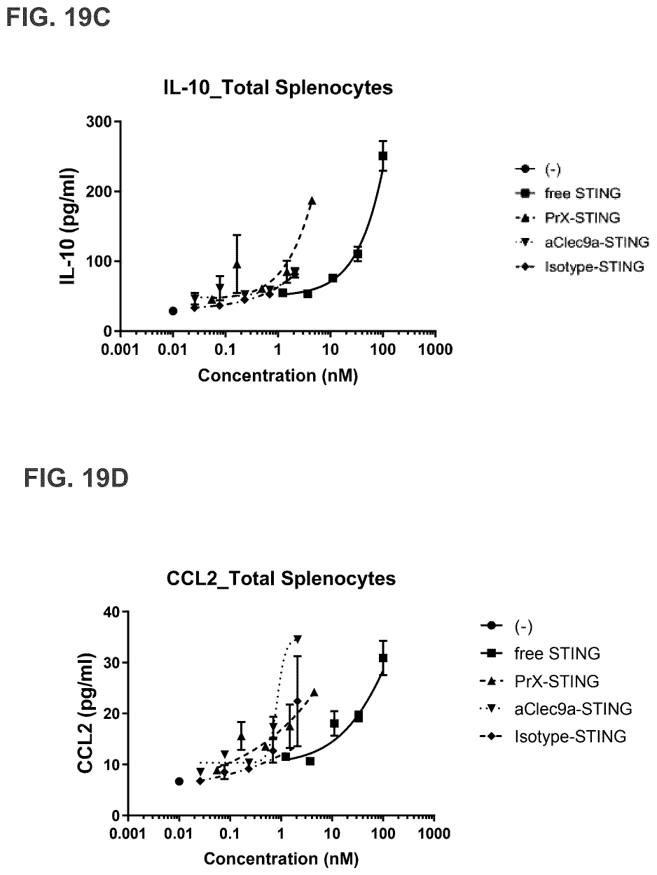

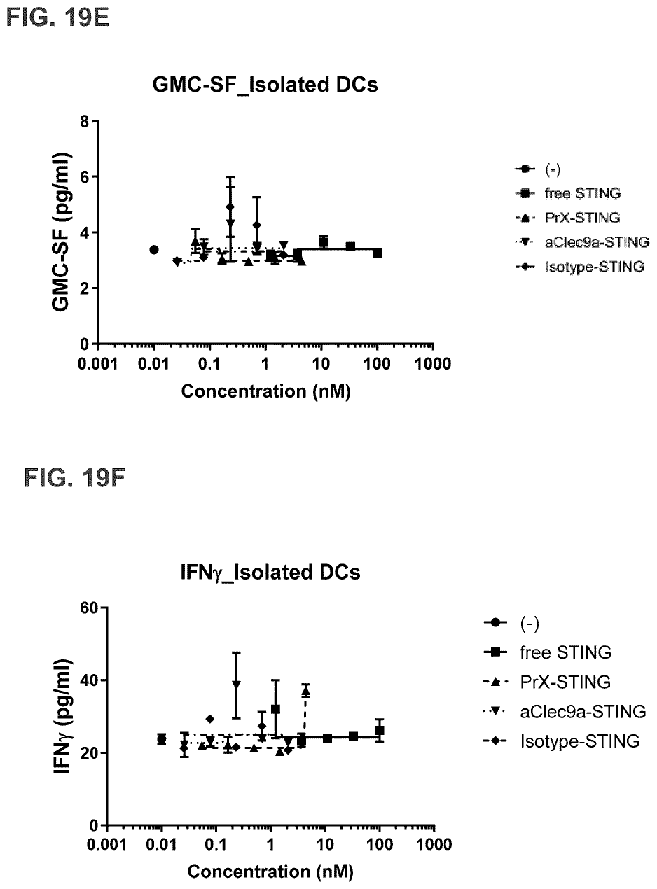

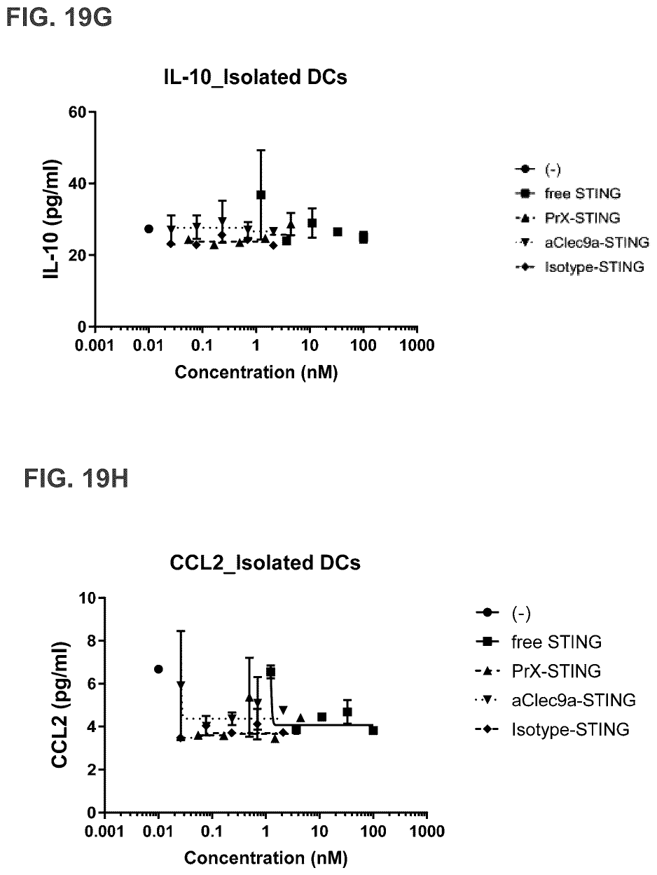

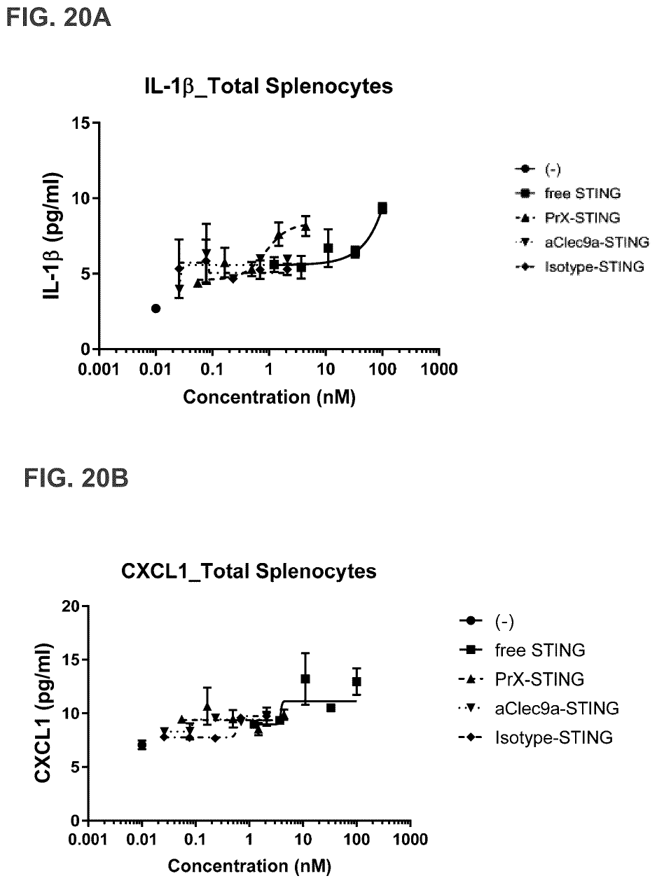

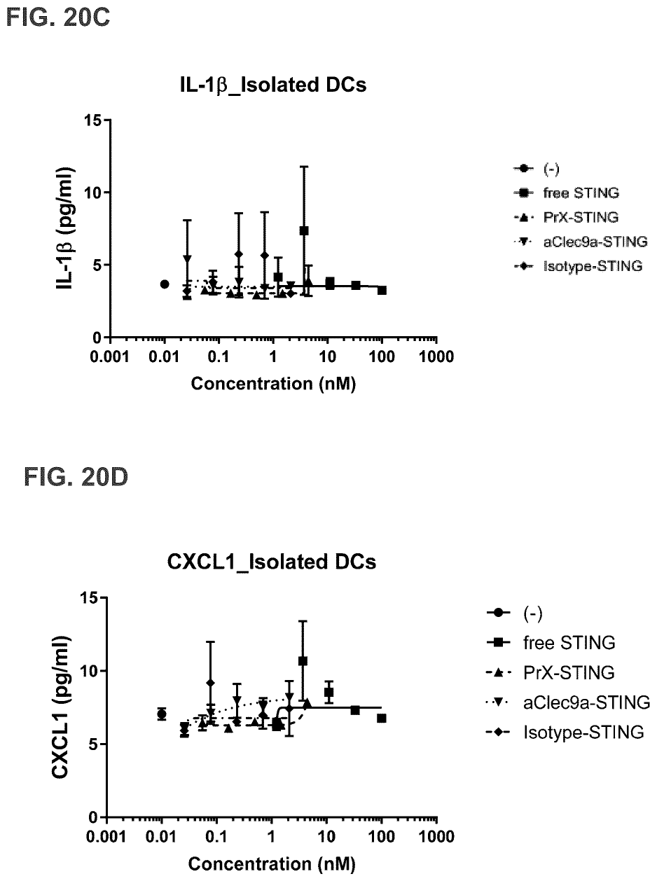

As shown in FIGS. 16B, 17D-17F, 18D-18F, 19E-19G, 20C, and 20D, isolated splenic DCs treated with anti-Clec9a exosomes loaded with STING agonist produced greater amounts of majority of the cytokines tested (e.g., CXCL10, CCL5, IL-6, IL-12, IFNα, TNFα). However, when total splenocytes were used, no significant differences were observed (see FIGS. 16A, 17A-17C, 18A-18C, 19A-19D, 20A, and 20B). This lack of difference further supports the specificity of anti-Clec9a targeting moieties, as Clec9a expressing cells make up a very low frequency of the total splenocytes.

Accordingly, the above results further confirm that anti-Clec9a targeting moieties can be used to specifically target EVs (e.g., exosomes), including those loaded with a payload, such as a STING agonist, to Clec9a-expressing cells.

### Example 9: In Vivo Analysis of Anti-Clec9a EVs (e.g., Exosomes) in a Tumor Model

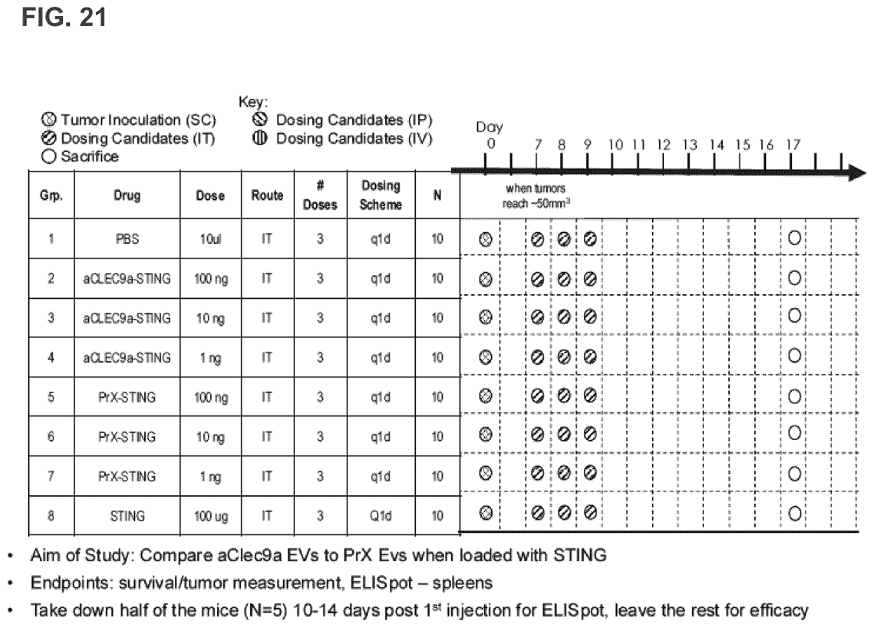

To determine whether the disclosed anti-Clec9a EVs (e.g., exosomes) loaded with STING agonist can be used to treat tumor in vivo, a tumor animal model will be used. As shown in FIG. 21, animals will be inoculated with tumor (subcutaneously) and then once the tumor has been established, the animals will be treated with one of the following regimens: (i) PBS alone; (ii) anti-Clec9a EVs (e.g., exosomes) loaded with STING agonist; (iii) STING-loaded EVs (e.g., exosomes) expressing Scaffold X alone; and (iv) soluble STING agonist. Different routes of administration will also be tested (e.g., intrathecal, intravenous, intraperitoneal). The animals will be periodically monitored and both tumor size and survival of the animals will be assessed. Some of the animals will also be sacrificed, and the anti-tumor response in different tissues will be assessed.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the disclosure(s). Many variations will become apparent to those skilled in the art upon review of this specification.

1. An extracellular vesicle (EV) comprising an exogenous targeting moiety that specifically binds to a marker for a dendritic cell.

2. The EV of claim 1, wherein the marker is present only on the dendritic cell.

3. The EV of claim 1, wherein the dendritic cell comprises a plasmacytoid dendritic cell (pDC), a myeloid/conventional dendritic cell 1 (cDC1), a myeloid/conventional dendritic cell 2 (cDC2), or any combination thereof.

4. (canceled)

5. The EV of claim 1, wherein the marker comprises a C-type lectin domain family 9 member A (Clec9a) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), or any combination thereof.

6. The EV of claim 5, wherein the marker comprises (i) an epitope in the Clec9a protein, or (ii) a C-type lectin like domain.

7-10. (canceled)

11. The EV of claim 1, wherein the exogenous targeting moiety comprises a peptide, an antibody or an antigen-binding fragment thereof, a chemical compound, or any combination thereof.

12-15. (canceled)

16. The EV of claim 1, wherein the EV comprises a scaffold protein linking the exogenous targeting moiety to the EV.

17. The EV of claim 16, wherein the scaffold protein is a Scaffold X protein.

18. The EV of claim 17, wherein the Scaffold X protein comprises prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, or any combination thereof.

19. (canceled)

20. (canceled)

21. The EV of claim 1, further comprising a Scaffold Y protein.

22. The EV of claim 21, wherein the Scaffold Y protein comprises myristoylated alanine rich Protein Kinase C substrate (the MARCKS protein), myristoylated alanine rich Protein Kinase C substrate like 1 (the MARCKSL1 protein), brain acid soluble protein 1 (the BASP1 protein), a fragment thereof, and or any combination thereof.

23-45. (canceled)

46. The EV of claim 1, further comprising a therapeutic molecule, an immune modulator, an adjuvant, or any combination thereof.

47-57. (canceled)

58. The EV of claim 1, wherein the EV is an exosome.

59. The EV of claim 46, wherein the therapeutic molecule is associated with a Scaffold X or a Scaffold Y protein.

60-64. (canceled)

65. A pharmaceutical composition comprising the EV of claim 1 and a pharmaceutically acceptable carrier.

66. A cell that produces the EV of claim 1.

67. (canceled)

68. A kit comprising the EV of claim 1 and instructions for use.

69. A method of making EVs comprising culturing the cell of claim 66 under a suitable condition and obtaining the EVs.

70. A method of preventing or treating a disease in a subject in need thereof, comprising administering to the subject the pharmaceutical composition of claim 65.

71. The method of claim 70, wherein the disease is selected from a cancer, a hemophilia, diabetes, a growth factor deficiency, an eye disease, a graft-versus-host disease (GvHD), an autoimmune disease, a gastrointestinal disease, a cardiovascular disease, a respiratory disease, an allergic disease, a degenerative disease, an infectious disease, fibrotic diseases, or any combination thereof.

72-74. (canceled)
